(19) United States
(12) Patent Application Publication
(10) Pub. No.: US 2021/0236657 A1
(43) Pub. Date: Aug. 5, 2021

(54) NERVE-SPECIFIC FLUOROPHORE FORMULATIONS FOR DIRECT AND SYSTEMIC ADMINISTRATION

(71) Applicants: OREGON HEALTH & SCIENCE UNIVERSITY, Portland, OR (US); OREGON STATE UNIVERSITY, Corvallis, OR (US)

(72) Inventors: Summer L. Gibbs, West Linn, OR (US); Connor W. Barth, Portland, OR (US); Wathah G. Alani, Beaverton, OR (US); Vidhiben Shah, Hillsboro, OR (US); Lei Wang, Portland, OR (US)

(73) Assignees: OREGON HEALTH & SCIENCE UNIVERSITY, Portland, OR (US); OREGON STATE UNIVERSITY, Corvallis, OR (US)

(21) Appl. No.: 17/266,549

(22) PCT Filed: Aug. 6, 2019
(86) PCT No.: PCT/US2019/045347

§ 371 Date: Feb. 5, 2021

Related U.S. Application Data
(60) Provisional application No. 62/715,189, filed on Aug. 6, 2018.

(51) Int. Cl. A61K 49/00; A61K 9/06; A61K 47/36; A61K 47/10; A61K 9/107; A61K 47/40; A61B 5/00; G01N 1/30

(52) U.S. Cl. CPC A61K 49/0028; A61K 9/06; A61K 47/36; A61K 47/10; G01N 1/30; A61K 9/1075; A61K 47/40; A61B 5/0071; A61K 49/0054

(57) ABSTRACT

Nerve-specific fluorophore formulations for direct or systemic administration are described. The formulations can be used in fluorescence-guided surgery (FGS) to aid in nerve preservation during surgical interventions.

### CROSS-REFERENCE TO RELATED APPLICATIONS

This application claims the benefit of priority to U.S. Provisional Application No. 62/715,189, filed Aug. 6, 2018, which is incorporated herein by reference in its entirety as if fully set forth herein.

### STATEMENT REGARDING FEDERALLY SPONSORED RESEARCH OR DEVELOPMENT

This invention was made with government support under grant R01EB021362 awarded by the National Institutes of Health. The government has certain rights in the invention.

### FIELD OF THE DISCLOSURE

The current disclosure provides nerve-specific fluorophore formulations for direct (local) or systemic administration. The formulations are used in fluorescence-guided surgery (FGS) to aid in nerve preservation during surgical interventions.

### BACKGROUND OF THE DISCLOSURE

Over 300 million surgeries are performed worldwide each year. Despite many recent advances in the treatment of cancer and other diseases, surgery remains the most effective treatment option for a number of diseases and injuries. The ultimate goal of surgery is to remove or repair tissues while minimizing comorbidities by preserving vital structures such as nerves and blood vessels. Recent technological advances including minimally invasive robot assisted laparoscopic surgery have improved outcomes and made it possible to perform difficult procedures robustly with minimal risk. Furthermore, preoperative three-dimensional imaging technologies such as magnetic resonance imaging (MRI) and computed tomography (CT) have vastly improved diagnostic accuracy, staging, and preoperative planning.

While advances have been made, identifying vital structures for preservation (e.g., nerves) or tissue for complete resection (e.g., tumors) during surgical procedures remains difficult. Nerve identification and sparing can be difficult intraoperatively due to variations in patient anatomy and often little ability for direct nerve visualization in the surgical field. Currently, intraoperative nerve detection is performed through a combination of naked eye visualization, palpation, and electromyographic monitoring. Several imaging modalities have been utilized in clinical studies for nerve detection including ultrasound, optical coherence tomography, and confocal endomicroscopy. However, these lack specificity, resolution, and wide-field imaging functionality, making it difficult to identify nerve tissues in real time. As a result, nerve damage continues to plague surgical outcomes. Iatrogenic nerve injury affects up to 63 million patients worldwide annually, causing acute and chronic pain as well as impairment or loss of motor and sensory function. Radical prostatectomy (RP), a surgical procedure involving removal of the entire prostate as a prostate cancer cure, is particularly plagued by nerve damage. Furthermore, while minimally invasive methods, such as robotic assisted RP, can achieve equivalent cancer control to open RP while resulting in decreased blood loss, lower transfusion rate, and faster convalescence, these advances provide no benefit in nerve-sparing outcomes and in fact, remove the ability to directly palpate the tissue.

An imaging modality capable of wide field, real time identification of nerve tissues intraoperatively would greatly benefit surgeons in nerve preservation and reduce rates of iatrogenic nerve injury, improving quality of life for patients post-surgery.

In addition to sparing nerves during surgical interventions, the ability to detect the edge of tumors is also incredibly important in clinical medicine. For example, post-surgical tumor margin status is one of the most important prognostic factors for local cancer recurrence and is considered the main measure of a tumor resection's success. Patients undergoing breast conserving surgery (BCS), the most common treatment option for patients with early stage breast cancer, are left with involved or close surgical margins 20-60% of the time, determined by pathological assessment following completion of the surgery. Involved or close margins require follow up re-excision surgery and result in negative patient outcomes.

The current intraoperative guidance techniques, or lack thereof, handicap surgeons' ability to successfully complete the goals of a procedure. Therefore, an imaging modality that can provide intraoperative guidance by highlighting important structures (e.g., nerves) or tissues (e.g., tumor) would greatly benefit surgical outcomes and significantly reduce comorbidities.

Fluorescence-guided surgery (FGS) has the potential to revolutionize surgery by enhancing visualization of specific tissues intraoperatively, effectively bridging the gap between preoperative imaging and surgical guidance. Using optical imaging of targeted fluorescent probes, FGS offers sensitive, real-time, wide-field imaging using compact imaging systems that are easily integrated into the operating room. Several imaging systems have already been developed for FGS applications and are in use clinically. See, for instance: Lee et al. Plastic and reconstructive surgery 126, 1472-1481 (2010); Tummers et al. European journal of surgical oncology: the journal of the European Society of Surgical Oncology and the British Association of Surgical Oncology 40, 850-858 (2014); Troyan et al. Annals of surgical oncology 16, 2943-2952 (2009); Ashitate et al. The Journal of surgical research 176, 7-13 (2012); Verbeek et al. The Journal of urology 190, 574-579 (2013); Gibbs-Strauss et al. Molecular imaging 10, 91-101 (2011); Hirche et al. Surgical innovation 20, 516-523 (2013); Gotoh et al. Journal of surgical oncology 100, 75-79 (2009); and Kitagawa et al. Anticancer research 35, 6201-6205 (2015). Utilizing near-infrared (NIR) light (650-900 nm), many of these systems can identify targeted tissue at millimeter to centimeter imaging depths due to the increased tissue penetration of light and minimal background autofluorescence at these wavelengths (FIG. 1). Additionally, the use of NIR light allows these systems to be implemented in the surgical field without affecting conventional white light visualization. FGS have been successfully utilized in a wide variety of clinical applications to improve surgical outcomes, including tumor resection, sentinel lymph node mapping, angiography, lymphography, and ureter and bile duct anatomic imaging. See, for instance: Kitai et al. Breast Cancer 12, 211-215 (2005); Peek et al. Future Oncol 13, 455-467 (2017); Jeschke et al. Urology 80, 1080-1086 (2012); Chang et al. Plastic and reconstructive surgery 132, 1305-1314 (2013); Yamamoto et al. Eur J Vasc Endovasc Surg 43, 426-432 (2012); Boni et al. Surg Endosc 29, 2046-2055 (2015); Stummer et al. Neurosurgery 42, 518-525; discussion 525-516 (1998); Stummer et al. Lancet Oncol 7, 392-401 (2006); van Dam et al. Nat Med 17, 1315-1319 (2011); van der Vorst et al. World J Gastrointest Surg 4, 180-184 (2012); AI-Taher et al. J Laparoendosc Adv Surg Tech A 26, 870-875 (2016); Ankersmit et al. Surgical innovation 24, 245-252 (2017); and Samkoe et al. Cancer Control 25, 1073274817752332 (2018). For example, FGS applications in glioma resection using 5-aminolevulinic acid (5-ALA) and its fluorescent metabolite protoporphyrin IX (PpIX) has significantly enhanced complete resection rates and revolutionized neurosurgical treatment of brain tumors over the past decade (Hadjipanayis et al. Neurosurgery 77, 663-673 (2015)).

While the promise of FGS has been demonstrated in a variety of clinical and preclinical applications over the past several decades, few efforts in clinical translation of new targeted imaging agents for FGS have been successful, largely due to the enormous regulatory challenge and cost of introducing diagnostic imaging agents into the clinic. Only four fluorescent contrast agents are clinically approved, three of which were grandfathered in from their application as colorimetric dyes prior to the widespread use of fluorescence imaging (Gibbs. Quantitative imaging in medicine and surgery 2, 177-187 (2012)). Of these four dyes, only two, indocyanine green (ICG) and methylene blue, emit NIR fluorescence and both are blood pool agents. There is thus a great need for the development and clinical translation of tissue-specific fluorescent contrast agents for FGS.

Systemically administered probes are subject to the body's biodistribution and clearance, which can mask binding due to low levels of accumulation of a microdose in the targeted tissue. One method for ensuring adequate binding in the tissue of interest and improving sensitivity is local or direct administration at the surgical site. By directly applying the fluorescent probe to the tissues of interest within the surgical field, selective labeling can be attained with a significantly lower dose than systemic administration, yielding equivalent to higher intensity fluorescence signal in the tissue of interest. Aside from significantly lowering the required dose for signal, direct (local) administration provides rapid and highly selective staining, which is beneficial for certain applications to avoid unwanted background that can be caused upon systemic administration.

FGS is well suited to aid in the preservation of vital nerve structures. Direct administration is an especially suited administration strategy for fluorescence-guided nerve sparing RP because nerve labeling via systemic administration would generate high background from nerves in the prostate, which are not able to be spared, and renal nerve-specific fluorophore clearance generates significant fluorescence in the urine within the adjacent bladder (Barth and Summer. Theranostics (2016); Tewari et al. BJU international 98, 314-323 (2006); Patel et al. Eur Urol 61, 571-576 (2012); Barth & Gibbs. Theranostics 7, 573-593 (2017)).

Currently, no NIR nerve-specific fluorophore exists and further fluorophore development is required to obtain a proper candidate for clinical translation. Several classes of nerve specific fluorophores have been studied for FGS. See, for instance: Gibbs-Strauss et al. Molecular imaging 10, 91-101 (2011); Wu et al. Journal of medicinal chemistry 51, 6682-6688 (2008); Wang et al. The journal of histochemistry and cytochemistry: official journal of the Histochemistry Society 58, 611-621 (2010); Gibbs et al. PloS one 8, e73493 (2013); Stankoff et al. Proceedings of the National Academy of Sciences of the United States of America 103, 9304-9309 (2006); Cotero et al. Molecular imaging and biology: MIB: the official publication of the Academy of Molecular Imaging 14, 708-717 (2012); Cotero et al. PloS one 10, e0130276 (2015); Bajaj et al. The journal of histochemistry and cytochemistry: official journal of the Histochemistry Society 61, 19-30 (2013); Gibbs-Strauss et al. Molecular imaging 9, 128-140 (2010); Meyers et al. The Journal of neuroscience: the official journal of the Society for Neuroscience 23, 4054-4065 (2003); Wang et al. The Journal of Neuroscience: the official journal of the Society for Neuroscience 31, 2382-2390 (2011); and Park et al. Theranostics 4, 823-833 (2014). Of these, Oxazine 4 is the most promising candidate for development, showing high nerve-specificity and red shifted absorption and emission spectra close to the NIR (Park et al. Theranostics 4, 823-833 (2014)).

FGS offers a rapid and accurate approach to intra-surgical margin assessment that does not compromise tissue integrity. Direct administration of tumor-specific fluorescent probes to resected specimens is an attractive alternative to systemic administration that incurs no risks of toxicity to the patient and would provide an extremely rapid route to clinical use. However, early efforts to stain tissues in this manner resulted in high amounts of non-specific uptake and poor tumor to normal tissue contrast. However, using a dual probe staining approach, containing a targeted and untargeted probe, non-specific uptake can be corrected and highly specific tumor signal achieved (Davis et al. Opt Lett 38, 5184-5187 (2013); Barth et al. Theranostics 7, 4722-4734 (2017)).

Additionally, direct administration methods can be utilized in retrospective analysis of excised tissues such as tumors for tumor margin detection, allowing for indirect, yet still rapid FGS and post-surgical diagnostics.

### SUMMARY OF THE DISCLOSURE

The current disclosure describes work undertaken to develop novel fluorescent probe formulations and local administration/direct application to a tissue of interest and systemic administration methods for FGS not subject to the drawbacks noted in the Background. This disclosure provides significant progress in the development and characterization of clinically viable administration methods and novel imaging probes for nerve identification and tumor margin detection.

Particular embodiments include a gel-based formulation including a nerve-specific fluorophore to facilitate improved signal to background ratio (SBR) for direct administration during surgery through reduction in background staining. In particular embodiments, the gel-based formulations are liquid at room temperature but become a viscous gel upon contact with body temperature. For nerve staining at a surgical site, this characteristic is useful because it diminishes the spread of the applied fluorophore, improving overall SBR through background reduction.

For the gel-based formulation for direct administration, all excipients are FDA approved for human use and the formulation increases the solubility of nerve-specific fluorophores (e.g., LGW1-08) to clinically relevant concentrations. These formulations can be applied directly to the tissue of interest, where they undergo Sol-Gel transition at the site of application. This property is important for the formulation syringe-ability and retention at the tissue of interest. In particular embodiments, the sol-gel transition occurs within, e.g., 1-2 minutes (or, less than 30 seconds) after application, which is important to its practical use in a surgical setting. Further, the formulation can be removed from the tissue easily by washing the tissue with a pharmaceutically acceptable irrigation solution such as saline. These formulations can be scaled-up and produced under GMP conditions.

Also disclosed are two additional clinically-relevant formulations including a nerve-specific fluorophore for systemic administration. One formulation for systemic administration includes cyclodextrin (the main excipient is (2-hydroxypropyl)-β-cyclodextrin) and the second formulation for systemic administration includes a DSPE-PEG micelle. The main excipient in this formulation is N-(methylpolyoxyethylene oxycarbonyl)-1,2-distearoyl-sn-glycero-3-phosphoethanolamine, sodium salt.

In formulations for systemic administration, all excipients are FDA approved for human use, and both formulations increase the solubility of nerve-specific fluorophores (e.g., LGW1-08) to a clinically relevant concentration. Both of the formulations provide clinically relevant pharmacokinetic profiles and clinically acceptable safety (acute toxicity study) profiles for nerve-specific fluorophores. The formulations developed for systemic administration produce the required clinical effect, specifically nerve-specific fluorophore accumulation in the nerve of interest at the desirable SBR. Furthermore, both formulations can be scaled-up and produced under GMP conditions.

### BRIEF DESCRIPTION OF THE DRAWINGS

Many of the drawings submitted herein are better understood in color. Applicant considers the color versions of the drawings as part of the original submission and reserve the right to present color images of the drawings in later proceedings.

FIG. 1. Absorption spectra of various tissue components. Due to low absorbance of all major components between 650 and 900 nm wavelengths the near-infrared (NIR) is the optimal medical optical imaging window. OxyHb=oxygenated hemoglobin, DeoxyHb=deoxyhemoglobin. FIG. reprinted from Chance, B. Annals of the New York Academy of Sciences 838, 29-45 (1998). With permission. Copyright Clearance Center Rightslink.

FIG. 2. Schematic of gel-formulation components, preparation, and characterization.

FIGS. 3A, 3B: Initial gel formulation screening. (FIG. 3A) Representative images and (FIG. 3B) quantified intensities and nerve signal to background (SBRs) for each concentration of Na Alginate and PEO-PPO-PEO (poly(ethylene oxide)-poly(propylene oxide)-poly(ethylene oxide)) triblock in 125 μM LGW1-08 gel formulations for initial screening and comparison to a co-solvent formulation. All images are representative of data collected for n=6 nerve sites per fluorophore. Images are displayed without normalization. All quantified data is presented as mean+/−standard deviation. FL=fluorescence. Nerve intensity data for each formulation was compared to the co-solvent formulation data to test for significance. Nerve SBR data for each formulation was compared to control unstained data to test for significance. *=p value <0.05, **=p value <0.01, ***=p value <0.001, ****=p value <0.0001.

FIG. 4: Gel formulation viscosity assessment. Schematic, representative images, and quantified tissue spread surface areas for the gel formulation viscosity assessment studies. Dotted lines indicate the area of tissue spread of each formulation in each representative image. Data is representative of n=3 reps per formulation and angle tested.

FIGS. 5A, 5B: Gel formulation direct administration staining parameter testing. (FIG. 5A) Representative brachial plexus and sciatic nerve images and (FIG. 5B) quantified intensity and nerve SBR ratios for each concentration and incubation time tested during staining parameter testing studies. Images are representative of n=6 nerve sites. Images are displayed without normalization. FL=Fluorescence; min=minute.

FIGS. 6A-6C: Gel formulation direct administration washing protocol testing. (FIG. 6A) Representative images and (FIG. 6B) quantified nerve intensities and SBRs for wash testing studies. All images are representative of data collected for n=6 nerve sites per wash temperature, with images collected at 0, 1, 2, 3, 4, 5, 6, 9, 12, 16, and 18 wash. (FIG. 6C) Nerve intensity and SBRs were calculated from images captured for 30 minutes following the final 18^{th} wash step to visualize the effects of clearance. All quantified data is presented as mean+/−standard deviation.

FIGS. 7A-7D. PEO-PPO-PEO triblock formulation toxicology testing. (FIG. 7A) blood marker, (FIG. 7B) electrolyte, and (FIG. 7C) hematological analysis results represented as concentrations (Units per liter (U/L), milligram per deciliter (mg/dL), millimole per liter (mmol/L), mole per microliter (M/μL), kilo per microliter (K/μL)) or percentages following administration of 22% PEO-PPO-PEO triblock either loaded with 100× microdose (1760 μg mouse) of LGW1-08 or unloaded. The dashed line boxes on each graph represent the normal expected range of values. (FIG. 7D) The measured mean weights for mice 14 days following administration of the loaded formulations vs. control un-administered animals. All data are representative of data collected for n=5 mice per formulation. All quantified data is presented as mean+/−standard deviation. M−1 d=measured values one day post injection; M−14 d=measured values 14 days post injection.

FIG. 8: Large animal direct administration formulation comparison. Color and fluorescence images captured of the swine iliac plexus stained via direct administration of either co-solvent or PEO-PPO-PEO triblock formulated Oxazine 4. Images of the staining method were captured during application of the formulation while color and fluorescence images were captured 2.5 hours following completion of the staining protocol.

FIG. 9. Schematic of systemic administration formulation components, preparation, and characterization.

FIGS. 10A-10D: Clinically viable systemic administration formulation initial screening. (FIG. 10A) Representative images and (FIG. 10C) quantification for Oxazine 4 using the varied formulation strategies in murine models. (FIG. 10B) Representative images and (FIG. 10D) quantification for LGW1-08 using the varied formulation strategies in murine models. All images are representative of data collected for n=12 nerve sites per formulation. All quantified data is presented as mean+/−standard deviation. N/M=Nerve to Muscle Ratio, N/A=Nerve to Adipose Ratio, Co-solv.=Co-solvent, Cyclod.=Cyclodextrin, DSPE-PEG=DSPE-PEG Micelles, F127=F127 Micelles, Lipo.=Liposomes. Nerve Intensity/s, N/M, and N/A data for each tested formulation was compared to the co-solvent formulation data to test for significance, where *=p value <0.05, **=p value <0.01, ***=p value <0.001, ****=p value <0.0001.

FIGS. 11A-11D: DSPE-PEG micelle and cyclodextrin toxicology testing. (FIG. 11A) Blood marker, (FIG. 11B) electrolyte, and (FIG. 11C) hematological analysis results represented as concentrations (Units per liter (U/L), milligram per deciliter (mg/dL), millimole per liter (mmol/L), mole per microliter (M/μL), kilo per microliter (K/μL)) or percentages following administration of the maximum tolerated dose (MTD, 3 mg/kg). The dashed line boxes on each graph represent the normal expected range of values. (FIG. 11D) The measured mean weights for mice 14 days following administration of the MTD. All data are representative of data collected for n=5 mice per formulation. All quantified data is presented as mean+/−standard deviation. M−1 d=measured values one day post injection; M−14 d=measured values 14 days post injection.

FIGS. 12A, 12B: DSPE-PEG micelle and cyclodextrin vs. co-solvent pharmacokinetics and biodistribution. (FIG. 12A) The blood LGW1-08 concentrations and release kinetics and (FIG. 12B) LCMS tissue biodistribution of DSPE-PEG micelles and cyclodextrin vs. co-solvent administered LGW1-08. All animals were systemically administered LGW1-08 at 2 mg/kg and blood was collected at 0, 1, 2, 4, 8, and 24 hours post injection. Organs were collected for biodistribution analysis at 0, 1, 2, 4, and 8 hours post injection All data is representative of n=5 mice per formulation. All quantified data is presented as mean+/−standard deviation. A=Terminal rate constant; t_{1/2}=elimination half-life; AUC_{inf}=total area under the plasma concentration-time curve; CL=total body clearance of drug from plasma; V_{z}=Volume of distribution during terminal phase.

FIGS. 13A-13C. DSPE-PEG micelle and cyclodextrin vs. co-solvent fluorescence pharmacokinetics and biodistribution. (FIG. 13A) Representative nerve images, (FIG. 13B) fluorescence signal intensities and nerve SBRs, and (FIG. 13C) fluorescence tissue biodistribution for co-solvent, DSPE-PEG micelle and cyclodextrin formulated LGW01-08. All animals were systemically administered LGW1-08 at 2 mg/kg and imaged at 0, 1, 2, 4, 8, and 24 hours post injection. All images are representative of data collected for n=12 nerve sites or n=5 mice per formulation. All quantified data is presented as mean+/−standard deviation.

FIGS. 14A-14C. DSPE-PEG micelle and cyclodextrin pharmacodynamic dose response. (FIG. 14A) tissue intensities, (FIG. 14B) nerve to muscle, and (FIG. 14C) nerve to adipose values for 0, 0.1, 0.2, 0.3, 0.6, 1, 1.5, 2, 2.5, and 3 mg/kg dose of LGW1-08 in DSPE-PEG micelles and cyclodextrin from images collected 2 hours following injection. In FIG. 14A, each set of bar graphs for each dose follows the order indicated in the legend: micelle nerve, micelle muscle, micelle adipose (for diamond set); and cyclodextrin nerve, cyclodextrin muscle, and cyclodextrin adipose (for star set). The means were calculated from n=12 nerve sites per dose. All quantified data is presented as mean+/−standard deviation.

FIG. 15. Chemical structures of an oxazine dye library.

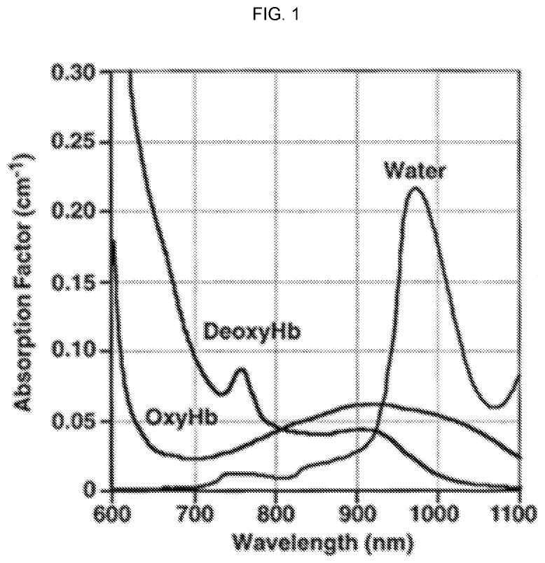

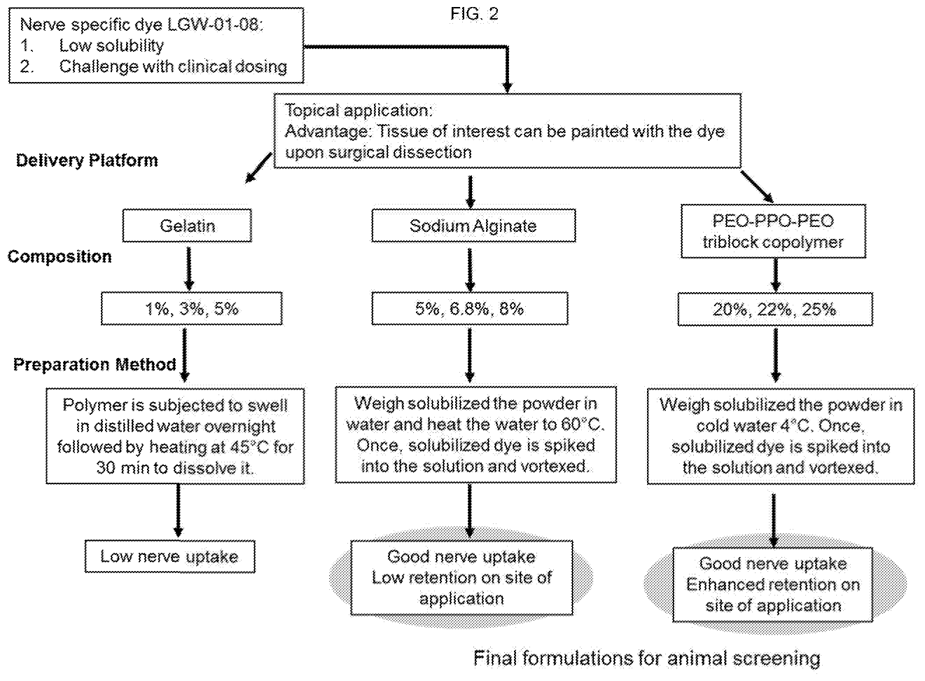

### DETAILED DESCRIPTION

Nerve damage plagues surgical outcomes, significantly affecting post-surgical quality of life. Despite the practice of nerve sparing techniques for decades, intraoperative nerve identification and sparing remains difficult and success rates are strongly correlated with surgeon experience level and ability to master the technique (Walsh & Donker. The Journal of urology 128, 492-497 (1982); Ficarra et al. Eur Urol 62, 405-417 (2012); Damber & Khatami. Acta oncologica 44, 599-604 (2005)). Fluorescence-guided surgery (FGS) shows promise for enhanced visualization of specifically highlighted tissue, such as nerves and tumor tissue, intraoperatively. FGS using optical imaging technology is capable of real-time, wide field identification of targeted tissues with high sensitivity and specificity from tissue targeted fluorescent probes. See, for instance: Frangioni. Journal of clinical oncology: official journal of the American Society of Clinical Oncology 26, 4012-4021 (2008); Gibbs. Quantitative imaging in medicine and surgery 2, 177-187 (2012); Gioux et al. Molecular imaging 9, 237-255 (2010); Vahrmeijer et al. Nature reviews. Clinical oncology 10, 507-518 (2013); and Nguyen et al. Nature reviews. Cancer 13, 653-662 (2013). Operating in the near-infrared (NIR) optical window (650-900 nm wavelengths) where tissue chromophore absorbance, autofluorescence and scattering are minimal, FGS technologies have the ability to identify targeted tissues at millimeter to centimeter depths against a black background (Chance. Annals of the New York Academy of Sciences 838, 29-45 (1998); Gibbs. Quantitative imaging in medicine and surgery 2, 177-187 (2012)).

Several imaging systems have been developed for FGS applications. see, for instance: Lee et al. Plastic and reconstructive surgery 126, 1472-1481 (2010); Tummers et al. European journal of surgical oncology: the journal of the European Society of Surgical Oncology and the British Association of Surgical Oncology 40, 850-858 (2014); Troyan et al. Annals of surgical oncology 16, 2943-2952 (2009); Ashitate et al. Real-time simultaneous near-infrared fluorescence imaging of bile duct and arterial anatomy. The Journal of surgical research 176, 7-13 (2012); Verbeek et al. The Journal of urology 190, 574-579 (2013); Gibbs-Strauss et al. Molecular imaging 10, 91-101 (2011); Hirche et al. Surgical innovation 20, 516-523 (2013); Gotoh et al. Journal of surgical oncology 100, 75-79 (2009); and Kitagawa et al. Anticancer research 35, 6201-6205 (2015); Importantly, the da Vinci surgical robot, frequently used for robotic assisted radical prostatectomy (RP), can be equipped with an FDA approved fluorescence imaging channel.

Direct administration (also sometimes referred to as local administration) is an attractive alternative to systemic administration of fluorescent probes for minimizing potential toxicity and easing regulatory burdens for first in human clinical studies. By selectively labeling tissues within the surgical field, direct administration requires a significantly lower dose than systemic administration. A direct administration methodology has been developed that provides equivalent nerve signal to background (SBR) to systemic administration following a 15-minute staining protocol. Barth & Gibbs. Theranostics 7, 573-593 (2017). This methodology has been successfully applied to autonomic nerve models, which closely mimic the nerves surrounding the prostate. This method has additional benefits in the application to RP since nerve labeling via systemic administration during RP would generate high background from nerves in the prostate, which are not able to be spared, and renal fluorophore clearance, producing significant fluorescence signal in the urine within the adjacent bladder. Both of these extraneous fluorescence signals would diminish the ability to identify the cavernous nerves within the neurovascular bundle (NVB), which are responsible for continence and potency (Barth and Summer. Theranostics (2016). Tewari et al. BJU international 98, 314-323 (2006); Patel et al. Eur Urol 61, 571-576 (2012)). Perhaps most importantly, the direct administration methodology requires 16 times lower dose than systemic administration and when scaled to humans by body surface area the dose falls within the requirements for clinical translation under an exploratory investigational new drug (eIND) application to the FDA. Studies conducted under an eIND require minimal preclinical toxicity testing, since only a microdose (<100 μg) is administered to each patient, significantly reducing the cost of first-in-human studies.

While the direct administration methodology has provided high nerve specificity and SBR with a short staining protocol in preclinical rodent models (Barth & Gibbs. Theranostics 7, 573-593 (2017)), preliminary staining studies in large animal models generated significant background. To facilitate clinical translation, an improved formulation strategy that is FDA approved and facilitates increased application control for staining a variety of tissue surfaces, angles, and morphologies will be required.

Several classes of nerve specific fluorescence imaging probes have been studied preclinically for FGS. See, for instance: Gibbs-Strauss et al. Molecular imaging 10, 91-101 (2011); Wu et al. Journal of medicinal chemistry 51, 6682-6688 (2008); Wang et al. The journal of histochemistry and cytochemistry: official journal of the Histochemistry Society 58, 611-621 (2010); Gibbs et al. PloS one 8, e73493 (2013); Stankoff et al. Proceedings of the National Academy of Sciences of the United States of America 103, 9304-9309 (2006); Cotero et al. Molecular imaging and biology: MIB: the official publication of the Academy of Molecular Imaging 14, 708-717 (2012); Cotero et al. PloS one 10, e0130276 (2015); Bajaj et al. The journal of histochemistry and cytochemistry: official journal of the Histochemistry Society 61, 19-30 (2013); Gibbs-Strauss et al. Molecular imaging 9, 128-140 (2010); Meyers et al. The Journal of neuroscience: the official journal of the Society for Neuroscience 23, 4054-4065 (2003); Wang et al. The Journal of neuroscience: the official journal of the Society for Neuroscience 31, 2382-2390 (2011); Park et al. Theranostics 4, 823-833 (2014). Of these, oxazine fluorophores (e.g., Oxazine 4) have demonstrated the most promise for clinical translation, with high nerve specificity following both direct and systemic administration. LGW1-08 is a particularly promising compound and was chosen as the lead compound for advancement to clinical studies. Although LGW1-08 has been shown to demonstrate high nerve specificity and adequate fluorescence signal for real time imaging, previous studies have been conducted utilizing a co-solvent formulation as a vehicle for intravenous injection (Gibbs-Strauss et al. Molecular imaging 10, 91-101 (2011); Barth & Gibbs. Theranostics 7, 573-593 (2017)). The co-solvent formulation is only stable at room temperature for <30 minutes, cannot solubilize concentrations above 5 mg/mL, and requires the use of dimethyl sulfoxide and Kolliphor EL as solubilizing agents, which hampers clinical translation due to vehicle induced toxicity issues. Additionally, the co-solvent formulation is liquid based and thus not ideal for staining angled or vertical tissue surfaces. Therefore, a clinically viable formulation with FDA approval was needed for direct administration and intravenous injection of nerve-specific fluorescence for FGS.

The current disclosure describes clinically relevant formulation strategies for direct administration and intravenous injection of LGW01-08 and other relevant compounds.

LGW1-08 is a near-infrared nerve specific oxazine fluorophore that shows promise in fluorescence imaging. The structure of LGW1-08 is the following. Its synthesis is described further below.

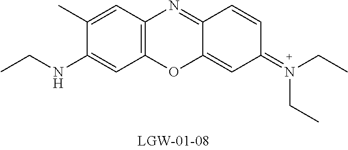

Additional fluorophores useful for nerve specific fluorescence imaging. In particular embodiments, the following compounds can be used in formulations and methods of the disclosure.

In particular embodiments, the fluorophores are compounds of Formula I:

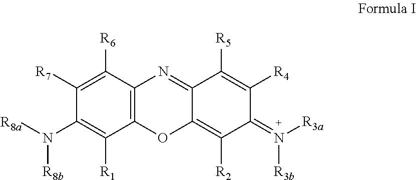

wherein:

R₁ and R₂ are each independently selected from hydrogen and C₁-C₄ alkyl;

R₃ₐ and R_{3b} are independently selected from hydrogen and C₁-C₄ alkyl, with the proviso that at least one of R₃ₐ and R_{3b} is not hydrogen; or R₃ₐ and R_{3b} together with the nitrogen atom to which they are bound form a 5- or 6-membered nitrogen-containing ring;

or R_{3b} is selected from hydrogen and C₁-C₄ alkyl, and R₃ₐ together with the nitrogen to which it is bound and R₄ forms a fused 5-membered or 6-membered ring, the 5-membered or 6-membered ring having one nitrogen heteroatom, two nitrogen heteroatoms, or one nitrogen heteroatom and one oxygen heteroatom, wherein each nitrogen heteroatom is optionally substituted by a C₁-C₄ alkyl substituent;

or R₃ₐ, the nitrogen to which it is bound, and R₄ form a fused 6-membered, nitrogen-containing ring and R_{3b}, the nitrogen to which it is bound, and R₂ together form a fused 6-membered, nitrogen-containing ring;

R₄ and R₇ are each independently selected from hydrogen, halogen, or C₁-C₄ alkyl;

R₅ and R₆ are each independently selected from hydrogen or C₁-C₄ alkyl;

R₈ₐ and R_{8b} are independently selected from hydrogen and C₁-C₄ alkyl, with the proviso that at least one of R₈ₐ and R_{8b} is not hydrogen; or R₈ₐ and R_{8b} together with the nitrogen to which they are bound form a 5- or 6-membered nitrogen-containing ring optionally substituted by 1, 2, 3, or 4 C₁-C₄ alkyl substituents;

or R_{8b} is selected from hydrogen and C₁-C₄ alkyl, and R₈ₐ together with the nitrogen to which it is bound and R₇ forms a fused 6-membered ring having one nitrogen heteroatom, two nitrogen heteroatoms, or one nitrogen heteroatom and one oxygen heteroatom, wherein each nitrogen heteroatom is optionally substituted by 1, 2, 3, or 4 C₁-C₄ alkyl substituents;

or R₈ₐ, the nitrogen to which it is bound, and R₇ form a fused 6-membered, nitrogen-containing ring and R_{8b}, the nitrogen to which it is bound, and R₁ together form a fused 6-membered, nitrogen-containing ring.

Also provided is a compound of Formula I, as defined above, with the proviso that the compounds of Formula I do not include N3,N3,N7,N7-tetraethyl-10H-phenoxazine-3,7-diamine; N3,N7-diethyl-10H-phenoxazine-3,7-diamine; N3,N3,N7,N7-tetramethyl-10H-phenoxazine-3,7-diamine; and N7,N7-diethyl-N3,N3,2-trimethyl-10H-phenoxazine-3,7-diamine.

In particular embodiments, the fluorophores are compounds of Formula I:

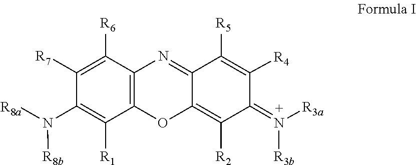

wherein:

R₁ and R₂ are each hydrogen;

R₃ₐ and R_{3b} are independently selected from hydrogen and C₁-C₄ alkyl, with the proviso that at least one of R₃ₐ and R_{3b} is not hydrogen; or R₃ₐ and R_{3b} together with the nitrogen atom to which they are bound form a 5- or 6-membered nitrogen-containing ring;

or R_{3b} is selected from hydrogen and C₁-C₂ alkyl, and R3a together with the nitrogen to which it is bound and R₄ forms a fused 6-membered ring having one nitrogen heteroatom, two nitrogen heteroatoms, or one nitrogen heteroatom and one oxygen heteroatom, wherein each nitrogen heteroatom is optionally substituted by a C₁-C₂ alkyl substituent;

R₄, R₅, R₆, and R₇ are each independently selected from hydrogen or C₁-C₂ alkyl;

R₈ₐ and R_{8b} are independently selected from hydrogen and C₁-C₂ alkyl, with the proviso that at least one of R₈ₐ and R_{8b} is not hydrogen; or R₈ₐ and R_{8b} together with the nitrogen to which they are bound form a 5- or 6-membered nitrogen-containing ring optionally substituted by 1, 2, 3, or 4 C₁-C₂ alkyl substituents;

or R_{8b} is selected from hydrogen and C₁-C₂ alkyl, and R₈ₐ together the nitrogen to which it is bound and R₇ forms a fused 6-membered ring having one nitrogen heteroatom, two nitrogen heteroatoms, or one nitrogen heteroatom and one oxygen heteroatom, wherein each nitrogen heteroatom is optionally substituted by 1, 2, 3, or 4 C₁-C₂ alkyl substituents;

or R₈ₐ, the nitrogen to which it is bound, and R₇ together form a fused 6-membered, nitrogen-containing ring and R_{8b}, the nitrogen to which it is bound, and R₁ together form a fused 6-membered, nitrogen-containing ring.

In particular embodiments, the fluorophores are compounds of Formula I:

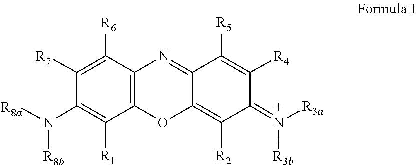

wherein:

R₁, R₂, R₄, R₅, R₆, and R₇ are each independently hydrogen or C₁-C₄ alkyl;

R₃ₐ and R_{3b} are each independently hydrogen or C₁-C₄ alkyl, with the proviso that at least one of R₃ₐ and R_{3b} is not hydrogen, or R₃ₐ and R_{3b} together with the nitrogen atom to which they are bound form a 5- or 6-membered nitrogen containing ring; and

R₈ₐ and R_{8b} are each independently hydrogen or C₁-C₄ alkyl, with the proviso that at least one of R₈ₐ and R_{8b} are not hydrogen, or R₈ₐ and R_{8b} together with the nitrogen atom to which they are bound form a 5- or 6-membered nitrogen containing ring.

In particular embodiments, the fluorophores are compounds of Formula I:

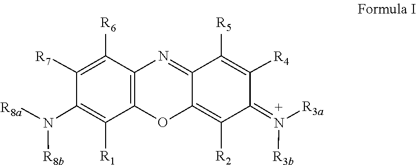

wherein:

R₁, R₂, R₄, R₅, R₆, and R₇ are each independently hydrogen or C₁-C₄ alkyl;

R₃ₐ and R_{3b} are each independently hydrogen or C₁-C₄ alkyl, with the proviso that at least one of R₃ₐ and R_{3b} is not hydrogen; and

R₈ₐ and R_{8b} together with the nitrogen atom to which they are bound form a 5- or 6-membered nitrogen containing ring.

In particular embodiments, the fluorophores are compounds selected individually from Formula I(a), Formula I(b), Formula I(c), Formula I(d), Formula I(e), and Formula I(f):

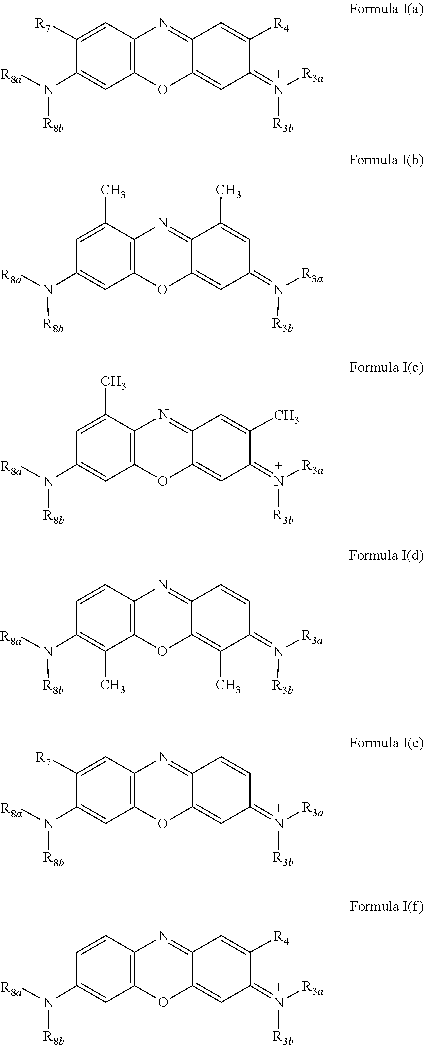

wherein:

R₄ and R₇ in each instance are independently selected from halogen and CH₃;

R₃ₐ and R_{3b} in each instance are independently selected from hydrogen and C₁-C₄ alkyl, with the proviso that at least one of R₃ₐ and R_{3b} is not hydrogen; or R₃ₐ and R_{3b} together with the nitrogen atom to which they are bound form a 5- or 6-membered nitrogen-containing ring;

or R_{3b} is selected from hydrogen and C₁-C₄ alkyl, and R₃ₐ together with the nitrogen to which it is bound and R₄ forms a fused 6-membered ring having one nitrogen heteroatom, two nitrogen heteroatoms, or one nitrogen heteroatom and one oxygen heteroatom, wherein each nitrogen heteroatom is optionally substituted by a C₁-C₄ alkyl substituent;

or R₃ₐ, the nitrogen to which it is bound, and R₄ form a fused 6-membered, nitrogen-containing ring and R_{3b}, the nitrogen to which it is bound, and R₂ together form a fused 6-membered, nitrogen-containing ring;

R₈ₐ and R_{8b} in each instance are independently selected from hydrogen and C₁-C₄ alkyl, with the proviso that at least one of R₈ₐ and R_{8b} is not hydrogen; or R₈ₐ and R_{8b} together with the nitrogen to which they are bound form a 5- or 6-membered nitrogen-containing ring optionally substituted by 1, 2, 3, or 4 C₁-C₄ alkyl substituents;

or R_{8b} is selected from hydrogen and C₁-C₄ alkyl, and R₈ₐ together with the nitrogen to which it is bound and R₇ forms a fused 6-membered ring having one nitrogen heteroatom, two nitrogen heteroatoms, or one nitrogen heteroatom and one oxygen heteroatom, wherein each nitrogen heteroatom is optionally substituted by 1, 2, 3, or 4 C₁-C₄ alkyl substituents; and

or R₈ₐ, the nitrogen to which it is bound, and R₇ form a fused 6-membered, nitrogen-containing ring and R_{8b}, the nitrogen to which it is bound, and R₁ together form a fused 6-membered, nitrogen-containing ring.

In particular embodiments, the fluorophores are compounds of Formula I(a):

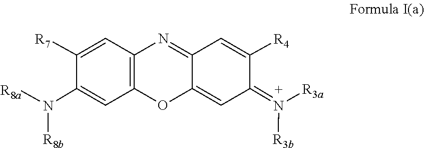

wherein R₄ and R₇ are independently selected from CH₃ and halogen; and each of R₃ₐ, R_{3b}, R₈ₐ, and R_{8b} are independently selected from hydrogen and C₁-C₄ alkyl.

In particular embodiments, the fluorophores are compounds of Formula I(e):

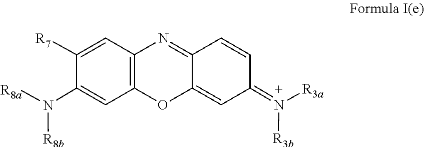

wherein R₇ is CH₃; and each of R₃ₐ, R_{3b}, R₈ₐ, and R_{8b} are independently selected from hydrogen and C₁-C₄ alkyl; with the proviso that at least one of R₃ₐ, R_{3b}, R₈ₐ, and R_{8b} is not hydrogen.

In particular embodiments, the fluorophores are compounds of Formula I(f):

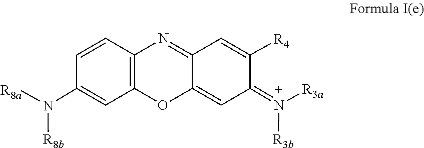

wherein R₄ is selected from CH₃ and hydrogen; R₈ₐ and R_{8b} together with the nitrogen to which they are bound form a 5- or 6-membered nitrogen-containing ring; and of R₃ₐ and R_{3b} are independently selected from hydrogen and C₁-C₄ alkyl; or R₃ₐ and R_{3b} together with the nitrogen to which they are bound form a 5- or 6-membered nitrogen-containing ring.

In particular embodiments, the fluorophores are compounds of Formula I(g):

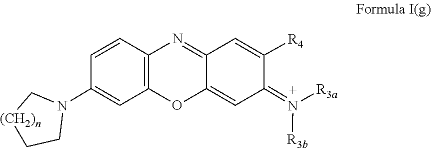

wherein:

R₃ₐ and R_{3b} are independently selected from hydrogen and C₁-C₄ alkyl; with the proviso that at least one of R₃ₐ and R_{3b} is C₁-C₄ alkyl; or R₃ₐ and R_{3b} together with the nitrogen to which they are bound form a 5- or 6-membered nitrogen-containing ring;

R₄ is selected from hydrogen and CH₃; and

n is an integer selected from 1 and 2.

It is understood in the description above that n=1 indicates the presence of a pyrrolidinyl ring and n=2 indicates a piperidinyl ring.

In particular embodiments, the fluorophores are compounds of Formula II:

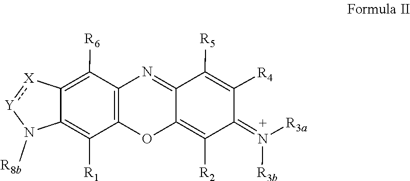

wherein:

R₁, R₂, R₄, R₅, and R₆ are each independently selected from hydrogen or C₁-C₄ alkyl;

X is selected from the group of —C(H)—, —CH₂—, —CH(C₁-C₄ alkyl)-, —C(C₁-C₄ alkyl)-, —N(H)—, —N(C₁-C₄ alkyl)-, and —O—;

Y is selected from —CH₂—, —CH₂—CH₂—, —CH₂—CH—, —CH(C₁-C₄ alkyl)-CH₂—, —C(C₁-C₄ alkyl)₂-CH₂—, —CH(C₁-C₄ alkyl)-CH—, and —C(C₁-C₄ alkyl)₂-CH—;

the dashed line ( [symbol] ) represents an optional double bond that exists when X is selected from —C(H)— or —C(C₁-C₄ alkyl)- and Y is selected from —CH₂—CH—, —CH(C₁-C₄ alkyl)-CH—, and —C(C₁-C₄ alkyl)₂-CH—;

R₃ₐ and R_{3b} are each independently hydrogen or C₁-C₄ alkyl, with the proviso that at least one of R₃ₐ and R_{3b} is not hydrogen, or R₃ₐ and R_{3b} together with the nitrogen atom to which they are bound form a 5- or 6-membered nitrogen containing ring; and

R_{8b} is selected from hydrogen and C₁-C₄ alkyl.

In particular embodiments, the fluorophores are compounds of Formula III:

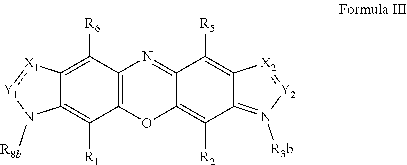

R₁, R₂, R₅, and R₆ are each independently selected from hydrogen and C₁-C₄ alkyl;

X₁ and X₂ are selected from —CH—, —CH₂—, —C(C₁-C₄ alkyl)-, —N(H)—, —N(C₁-C₄ alkyl)-, and —O—;

Y₁ and Y₂ are independently selected from —CH—, —CH₂—, —CH₂—CH₂—, —CH₂—CH—, —CH(C₁-C₄ alkyl)-CH₂—, —C(C₁-C₄ alkyl)₂-CH₂—, —CH(C₁-C₄ alkyl)-CH—, and —C(C₁-C₄ alkyl)₂-CH—;

the dashed line ( [symbol] ) in each instance represents and optional double bond when X₁ or X₂ is —C(H)— or —C(C₁-C₄ alkyl)- and Y₁ or Y₂ is selected from —CH—, —CH₂—CH—, —CH(C₁-C₄ alkyl)-CH—, and —C(C₁-C₄ alkyl)₂-CH—; and

R_{3b} and R_{8b} are each independently selected from hydrogen and C₁-C₄ alkyl.

In particular embodiments, the fluorophores are compounds of Formula IV:

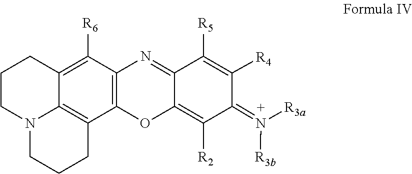

wherein:

R₂, R₄, R₅, and Re are each independently selected from hydrogen and C₁-C₄ alkyl; and

R₃ₐ and R_{3b} are each independently hydrogen or C₁-C₄ alkyl, with the proviso that at least one of R₃ₐ and R_{3b} is not hydrogen, or R₃ₐ and R_{3b} together with the nitrogen atom to which they are bound form a 5- or 6-membered nitrogen containing ring.

In particular embodiments, the fluorophores are compounds of Formula V:

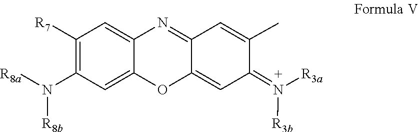

wherein:

R₃ₐ is C₁-C₄ alkyl;

R_{3b} is selected from H and C₁-C₄ alkyl; and

a) R₇ is hydrogen; and R₈ₐ and R_{8b}, along with the nitrogen atom to which they are bound, form a 5-membered saturated ring containing one nitrogen heteroatom or a 6-membered saturated ring containing one nitrogen heteroatom; or

b) R₇ and R₈ₐ, along with the nitrogen atom to which R₈ₐ is bound, form a 6-membered saturated or partially unsaturated ring containing one nitrogen heteroatom, the ring being optionally substituted by 1, 2, 3, or 4 C₁-C₄ alkyl substituents; and R_{8b} is selected from H and C₁-C₄ alkyl.

In particular embodiments, the fluorophores are compounds of Formula VI:

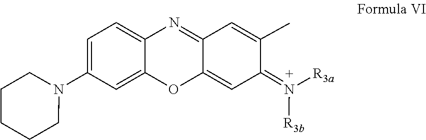

wherein R₃ₐ is C₁-C₄ alkyl; and R_{3b} is selected from H and C₁-C₄ alkyl.

In particular embodiments, the fluorophores are compounds of Formula VII:

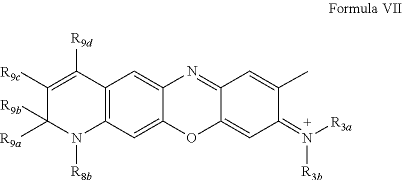

wherein R₃ₐ is C₁-C₄ alkyl; R_{3b} is selected from H and C₁-C₄ alkyl; R_{8b} is selected from H and C₁-C₄ alkyl; and each of R₉ₐ, R_{9b}, R_{9c}, and R_{9d} are independently selected from H and methyl.

In particular embodiments, the fluorophores are compounds of Formula VIII:

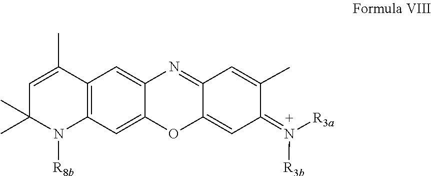

wherein R₃ₐ is C₁-C₄ alkyl; R_{3b} is selected from H and C₁-C₄ alkyl; R_{8b} is selected from H and C₁-C₄ alkyl.

Gel-Based Formulation for Direct Administration.

Gelatin is an animal based protein produced from collagen. The intermolecular bonds in gelatin mainly consist of hydrogen bonds which make the gel thermally reversible. The gel melts below human body temperature making it ideal for injecting it through tubes. Solutions with three concentrations (1.0, 3.0 and 5.0%, w/v) of gelatin were prepared by allowing the gelatin to swell in distilled water overnight (15 h) followed by heating at 45° C. for 30 min to dissolve it. Once in solution, the solubilized dye is spiked into the solution and vortexed. However, the solution was too viscous to let the dye molecule pass through the polymer, leading to low nerve staining.

F127 (Poloxamer 407) has been studied most extensively as a drug delivery system since it has the least toxicity in the commercially available nonionic triblock copolymers composed of a centrail hydrophobic chain of polyoxypropylene flanked by two hydrophilic chains of poly oxyethylene (PLURONIC® series). F127 is more soluble in cold water than in hot water as a result of increased solvation and hydrogen bonding at lower temperatures. At 20% or higher w/w concentration in water, F127 aqueous solutions are liquid at refrigerated temperatures (4-5° C.) but converts to gel upon warming to room temperature. The gelation is reversible upon cooling. This phenomenon suggests that when applied directly or injected into a body cavity or subcutaneously, the gel preparation will form a solid artificial barrier and a sustained release depot. The unique sol-gel-sol transition behavior makes this system a very attractive drug delivery system for painting the tissue of interest. Three different concentrations (20%, 22%, 25%) F127 gels were evaluated and based on animal screening it was found that 22% F127 showed optimum retention at the tissue site with ideal nerve uptake. Particular embodiments can utilize a PEO-PPO-PEO (poly(ethylene oxide)-poly(propylene oxide)-poly(ethylene oxide)) triblock copolymer with a molecular weight of 12,000-13,000 Dalton. Particular embodiments can utilize a PEO-PPO-PEO triblock copolymer with a molecular weight of 12,600 Dalton. In particular embodiments, a formulation including a fluorophore for direct administration can include a concentration of PEO-PPO-PEO triblock copolymer at a range of 18% to 26%, at a range of 19% to 25%, at a range of 20% to 24%, and at a range of 21% to 23%. In particular embodiments, a formulation for direct administration can include a concentration of PEO-PPO-PEO triblock copolymer at 18%, 19%, 20%, 21%, 22%, 23%, 24%, 25%, and 26%, and all values in between. In particular embodiments, a formulation for direct administration can include a concentration of PEO-PPO-PEO triblock copolymer at 22%. In particular embodiments, a gel-based formulation for tissue imaging includes (i) 18-26% PEO-PPO-PEO triblock copolymer; (ii) 19-25% PEO-PPO-PEO triblock copolymer; (iii) 20-24% PEO-PPO-PEO triblock copolymer; and (iv) 21-23% PEO-PPO-PEO triblock copolymer.

F-127 is an FDA approved polymer and its use within the current disclosure is novel because of the following reasons:
i) PEO-PPO-PEO triblock based gel has never been used to paint a tissue site of interest.
ii) The formulation increases solubility as well as permeability of a nerve-specific fluorophore.
iii) The formulation provides ideal viscosity for maximum retention of the nerve-specific fluorophore on the site of application without running out.
iv) The formulation produces similar nerve to muscle ratio as the co-solvent formulation, which being toxic is not applicable in clinic.

Thus, disclosed herein is the development of a clinically viable gel-based formulation strategy that enables direct administration of nerve-specific fluorescent contrast agents with increased control for nerve sparing FGS applications. A PEO-PPO-PEO triblock (e.g., F127 Pluronic®) formulation was used to solubilize a novel near-infrared nerve specific oxazine fluorophore, LGW1-08, providing increased staining control for a variety of tissue surfaces, angles, and morphologies. Additionally, the formulation developed herein possesses unique gelling characteristics, allowing it to easily be spread as a liquid followed by rapid gelling at body temperature for subsequent tissue hold. Further analysis of the direct administration protocol has decreased the time to complete staining to a total of 1-2 minutes. The resulting gel formulation and direct administration methodology provides an ideal platform for clinical translation of novel nerve-specific fluorophores for FGS.

Alginates have been used as versatile bio-polymers for numerous applications. Alginates have conventionally been used as thickening, gel-forming and stabilizing agents. Alginate has a unique property of gelling in the presence of divalent ions such as calcium. 5.0%, 6.8% and 8% were evaluated and when screened for maximum retention at the application site, 6.8% was ideal but compared to PEO-PPO-PEO triblock it performed poorly in terms of the desired retention at the tissue site. In particular embodiments, a formulation including a fluorophore for direct administration can include a concentration of sodium alginate at a range of 5% to 10%, at a range of 5% to 9%, at a range of 5% to 8%, at a range of 6% to 8%, and at a range of 6% to 7%. In particular embodiments, a formulation for direct administration can include a concentration of sodium alginate at 5%, 6%, 7%, 8%, 9%, and 10%, and all values in between. In particular embodiments, a formulation for direct administration can include a concentration of sodium alginate at 6.5%. In particular embodiments, a gel-based formulation for tissue imaging includes (i) 5-10% sodium alginate; (ii) 5-9% sodium alginate; (iii) 5-8% sodium alginate; (iv) 6-8% sodium alginate; and (v) 6-7% sodium alginate. In particular embodiments, a formulation including a fluorophore for direct administration can include 5-10% alginate salt. In particular embodiments, an alignate salt includes calcium alginate, potassium alginate, magnesium alginate, and ammonium alginate.

Also provided is a gel-based formulation for tissue imaging including (i) a fluorophore and (ii) 5-8% sodium alginate and/or 20-24% PEO-PPO-PEO block copolymer.

Further provided is a gel-based formulation for tissue imaging including (i) a fluorophore and (ii) 6-7% sodium alginate and/or 20-24% PEO-PPO-PEO block copolymer.

Further provided is a gel-based formulation for tissue imaging including (i) a fluorophore and (ii) 6-7% sodium alginate and/or 21-23% PEO-PPO-PEO block copolymer.

Accordingly, a PEO-PPO-PEO triblock based formulation and/or a sodium alginate based formulation strategy is preferred to replace the laboratory grade co-solvent formulation used for initial development of nerve-specific fluorophores described herein.

Systemic Administration.

To maintain nerve-specificity, the molecular weight of fluorophore must remain low enough to permit passing through the blood-nerve-barrier following systemic administration. Thus, although LGW01-08 has demonstrated high nerve specificity and adequate fluorescence signal for real time imaging following systemic administration, the fluorophore's lipophilicity necessitates formulation as a vehicle for intravenous injection. Gibbs-Strauss et al. Molecular imaging 10, 91-101 (2011); Barth & Gibbs. Theranostics 7, 573-593 (2017).

Formulations for Systemic Administration.

Based on the solubility of the nerve specific dye, liposomes sphingomyelin cholesterol (55:45), a FDA approved lipid mix was considered first for encapsulation of the agent as the lipid vesicles have both the lipid bilayer and hydrophilic core. Two common methods for preparation of liposomes are passive and pH gradient method. For passive loading method, both LGW01-08 and lipids were dissolved in a mixture of chloroform and methanol (70:30) and subjected to rotary evaporation under vacuum. Upon achieving a thin film, the film was hydrated in citrate buffer at 200 rpm at 60° C. for an hour, after which the solution was extruded at 60° C. using Avanti extruder for 20 passes to obtain unilamellar vesicles. The vesicles were then passed through a column to separate the unencapsulated dye and stored at 4° C. till further use. However, as expected poor loading was achieved with the passive loading method.

To achieve better loading, pH gradient was achieved by using ammonium sulphate buffer instead of citrate buffer and the internal pH of the vesicle was made acidic by passing the lipid solution through a column pre-equilibrated with HEPES buffer pH 7.4. This method increased the loading to 0.26 mg/mL, which was around the solubility of LGW1-08 (0.25 mg/mL). Thus, liposomal formulation was not successful in increasing the solubility of LGW01-08.

The next delivery platform which could increase the solubility of the nerve dye without compromising the nerve specificity was micelles. They are 10-100 nm in size and include a core and shell structure; the inner core is the hydrophobic part of the block copolymer, which encapsulates the poorly water-soluble drug, whereas the outer shell formed by the hydrophilic block of the copolymer protects the drug from the aqueous environment and stabilizes the micelle against recognition in vivo by the reticuloendothelial system. Commercially available (A-B-A block), PLURONIC® ethylene oxide-propylene oxide block co-polymer capable of self-assembly to form micelle was evaluated for encapsulating the nerve dye. The size and hydrophobic blocks in the copolymer influence the loading of the molecule in the micelles. Based on previous experience, the PEO-PPO-PEO triblock was weighed and dissolved in acetone to which the dye pre-dissolved in acetone was added. The mixture was subjected to rotary evaporation under vacuum. Upon achieving a thin film, the film was hydrated in saline and centrifuged at 7500 g for 10 minutes. The supernatant was passed through a 0.22μ filter and analyzed for nerve dye loading. The solubility was increased almost 2-fold and a encapsulation of 0.46 mg/mL was achieved.

In order to achieve higher encapsulation, two more polymeric micelle platforms: Poly(ethylene glycol)(2 k)-block-poly(D,L-lactic acid) (1.8 k) (PEG-b-PLA) and Distearyl-phosphatidylethanolamine-PEG2000 (DSPE-PEG) were evaluated. Both the micelles were prepared using the solvent casting method. Briefly, 2 mg of LGW01-08 and 60 mg of DSPE-PEG lipid/60 mg PEG-PLA dissolved in 2 mL acetonitrile were combined in a 25 mL round bottom flask, which was evaporated under reduced pressure to form a thin dye distributed polymeric lipid film. The film was rehydrated in 2 mL of normal saline water by vortexing, centrifuged and filtered using a 0.22 micron nylon filter. Micelles were characterized for drug loading by using multiskan spectrum spectrophotometer (Thermo Fischer, Waltham, Mass.). Triplicate samples were prepared for quantification by diluting the complex 100-fold in 10% triton solution. The encapsulation achieved for DSPE-PEG micelle was higher than the PEG-PLA micelles. The solubility of the nerve dye was increased almost 3 folds in the DSPE-PEG micelles.

The goal was to have multiple formulations for animal nerve specificity screening and thus one additional FDA approved polymer, hydroxy-propyl-β cyclodextrin (HP-β-CD) was evaluated for loading of the nerve dye. Cyclodextrin can enhance the aqueous solubility of poorly water soluble molecules and at the same time increase the ability to permeate through biological membranes. Particularly, hydroxypropyl derivatives have shown to increase solubility, stability and bioavailability of numerous molecules.

HP-β-CD complex was prepared by the solvent casting method. Briefly, 2 mg of LGW01-08 and 60 mg of HP-β-CD was dissolved in 2 mL of 95% ethanol, which was evaporated under reduced pressure to form a thin dye distributed HP-β-CD film. The HP-β-CD complex was achieved by rehydration of the thin film with normal saline, centrifuged and filtered through 0.22 micron nylon filter. The LGW01-08 loading in the complex was quantified by photometric analysis at 638 nm using multiskan spectrum spectrophotometer (Thermo Fischer, Waltham, Mass.). Triplicate samples were prepared for quantification by diluting the complex 100-fold in 10% triton solution.

In particular embodiments, a formulation for systemic administration includes a fluorophore encapsulated by DSPE-PEG micelles at a concentration of 0.5 mg/mL to 0.9 mg/mL, 0.5 mg/mL to 1.2 mg/mL, or 0.7 mg/mL to 1.0 mg/mL. In particular embodiments, a formulation for systemic administration includes a fluorophore encapsulated by DSPE-PEG micelles at 0.5 mg/mL, 0.6 mg/mL, 0.7 mg/mL, 0.8 mg/mL, 0.9 mg/mL, 1.0 mg/mL, 1.1 mg/mL, and 1.2 mg/mL. In particular embodiments, a formulation for systemic administration includes a fluorophore encapsulated by DSPE-PEG micelles at 0.7 mg/mL. In particular embodiments, a formulation for systemic administration includes a fluorophore encapsulated by cyclodextrin at a concentration of 0.5 mg/mL to 0.9 mg/mL, 0.5 mg/mL to 1.2 mg/mL, or 0.7 mg/mL to 1.0 mg/mL. In particular embodiments, a formulation for systemic administration includes a fluorophore encapsulated by cyclodextrin at 0.5 mg/mL, 0.6 mg/mL, 0.7 mg/mL, 0.8 mg/mL, 0.9 mg/mL, 1.0 mg/mL, 1.1 mg/mL, and 1.2 mg/mL. In particular embodiments, a formulation for systemic administration includes a fluorophore encapsulated by cyclodextrin at 0.7 mg/mL.

Both a cyclodextrin and DSPE-PEG micelle-based formulation strategy are preferred to replace the laboratory grade co-solvent formulation used for initial nerve-specific fluorophore development. Both of these formulation strategies are FDA approved and are novel because of the following reasons:
i) Both increase the solubility of nerve specific dye
ii) They produce similar nerve to muscle ratio as the co-solvent formulation, which being toxic is not applicable in clinic.
iii) They do not modify the pharmacokinetic property of the dye resulting in circulation half-life of the dye to be around 12 h, ideal for the clinical application of the molecule.

The disclosed formulations for delivery of fluorophores have improved stability compared to previously employed co-solvent formulations. Additionally, toxicity testing, pharmacokinetics, and dose ranging studies have been carried out to determine important pharmacological properties of the novel fluorophore/formulation combinations. The work presented herein provides an extensive framework for clinical translation of this promising nerve specific contrast agent to improve nerve and tissue identification intraoperatively.

Chemical Synthesis.

LGW-01-08.

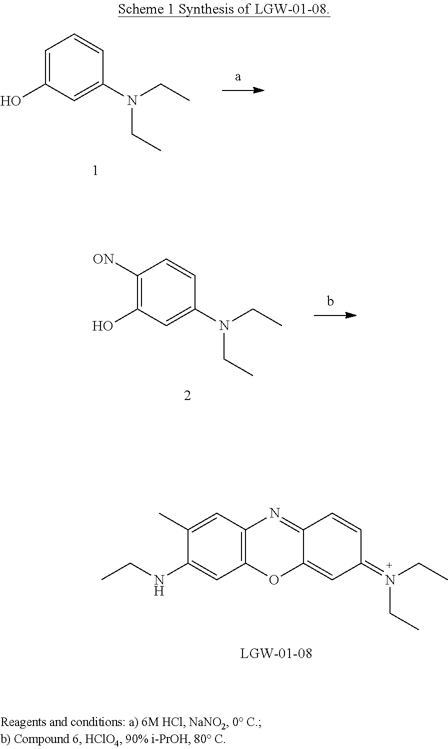

### 5-(diethylamino)-2-nitrosophenol (2)

Compound 1 (10 g, 60.52 mmol) was dissolved in an ice-cold 6 M HCl solution (50 mL). To the solution above, NaNO₂ (4.26 g, 61.73 mmol) was added portion wise over 1 h while maintaining the temperature of the solution below 5° C., such that no brown NOx vapors were observed. The reaction mixture was stirred for additional 2 h. After which, the precipitate was filtered through a Büchner funnel and washed with small portions of ice-cold 2 M HCl solution. The product was left in the funnel and air dried overnight to afford compound 2 (9.67 g, 82%) as a yellow solid, which was used for the next step without further purification.

### N-ethyl-N-(7-(ethylamino)-8-methyl-3H-phenoxazin-3-ylidene)ethanaminium (LGW-01-08)

Compound 6 (150 mg, 0.99 mmol) was dissolved in a solution of i-PrOH/H₂O (9/1, 2 mL) at 80° C. for 30 min. A suspended solution of 2 (202 mg, 1.04 mmol) and HClO₄ (70%, 90 μL) in 90% i-PrOH (3 mL) was added into the solution above in 4 portions over 1 h. The resulting solution was stirred overnight. During which time, the color of the reaction mixture changed from light brown to green and finally dark blue. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-01-08 (254 mg, 72%) as a dark green solid.

LGW-01-18.

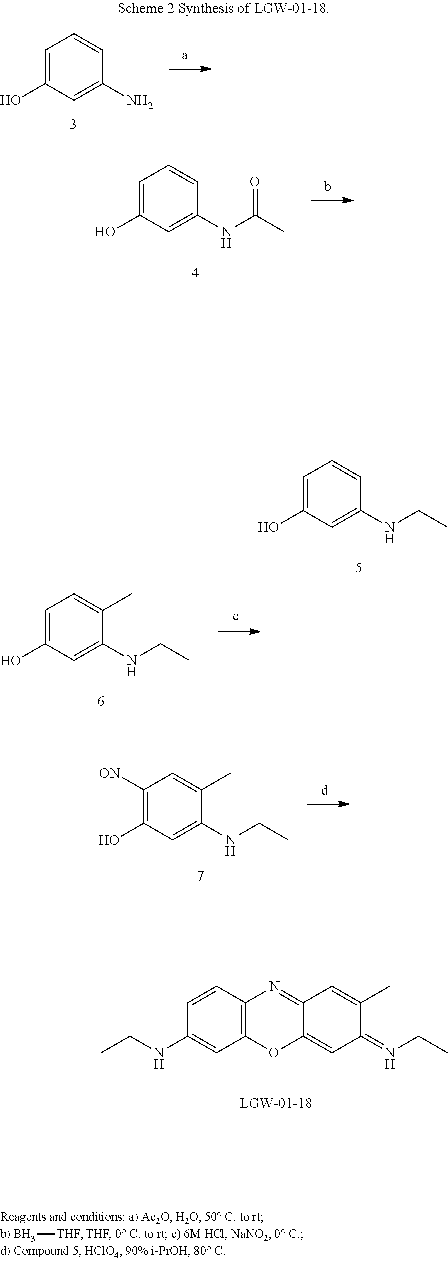

### N-(3-hydroxyphenyl)acetamide (4)

Compound 3 (1 g, 9.16 mmol) was suspended in 10 mL DI water, to which Acetic anhydride (2.60 mL, 27.49 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of DI water. The product was left in the funnel and air dried overnight to afford Compound 4 (1.19 g, 86%) as a light gray solid, which was used for the next step without further purification.

### 3-(ethylamino)phenol (5)

A solution of 4 (1 g, 6.62 mmol) in anhydrous THF (20 mL) was stirred in an ice bath under N₂ for 30 mins. Borane tetrahydrofuran complex solution (1 M, 20 mL) was added to the solution above using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, and the residue was purified by flash column chromatography with silica gel (25 g), using DCM/Hexane as eluent to obtain 5 (832 mg, 92%) as a black solid.

### 5-(ethylamino)-4-methyl-2-nitrosophenol (7)

Compound 6 (10 g, 66.13 mmol) was dissolved in an ice-cold 6 M HCl solution (50 mL). To the solution above, NaNO₂ (4.79 g, 69.44 mmol) was added portion wise over 1 h while maintaining the temperature of the solution below 5° C., such that no brown NOx vapors were observed. The reaction mixture was stirred for additional 2 h. After which, the precipitate was filtered through a Büchner funnel and washed with small portions of ice-cold 2M HCl solution. The product was left in the funnel and air dried overnight to afford compound 7 (9.42 g, 79%) as a yellow solid, which was used for the next step without further purification.

LGW-01-21

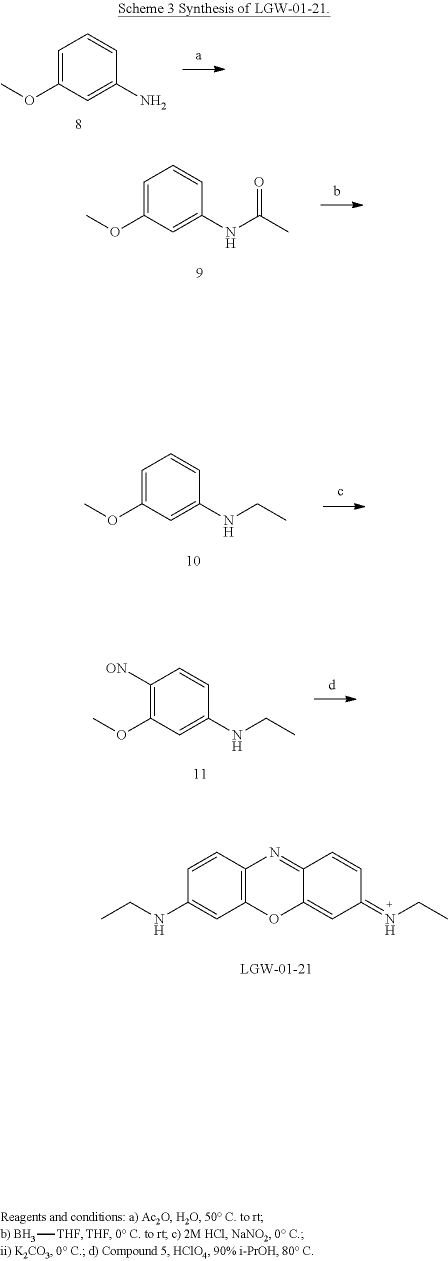

### N-(3-methoxyphenyl)acetamide (9)

Compound 8 (0.91 mL, 8.12 mmol) was suspended in 10 mL DI water, to which Acetic anhydride (2.30 mL, 24.36 mmol) was added dropwise. The reaction mixture was placed in an ultrasonication bath for 1 min, then was stirred in a water bath (50° C.) for 10 min. The resulting clear solution was stirred overnight at rt. After which, the solution was diluted with 50 mL DI water, and solid K₂CO₃ was added until the pH value of the solution rose above 8. The white precipitate was collected via vacuum filtration and washed with small portions of DI water. The product was left in the funnel and air dried overnight to afford compound 9 (1.27 g, 95%) as a light gray solid, which was used for the next step without further purification.

### N-ethyl-3-methoxyaniline (10)

A solution of 9 (1 g, 6.05 mmol) in anhydrous THF (18 mL) was stirred in an ice bath under N₂ for 30 mins. Borane tetrahydrofuran complex solution (1 M, 18 mL) was added to the solution above using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, and the residue was purified by flash column chromatography with silica gel (25 g), using DCM/Hexane as eluent to give 10 (878 mg, 96%) as a clear liquid.

### N-ethyl-3-methoxy-4-nitrosoaniline (11)

Compound 10 (0.5 g, 3.31 mmol) was dissolved in an ice-cold 2 M HCl solution (10 mL). To the solution above, NaNO₂ (0.25 g, 3.64 mmol) was added portion wise over 1 h while maintaining the temperature of the solution below 5° C., such that no brown NOx vapors were observed. The reaction mixture was stirred for additional 2 h. The solution was carefully basified with solid K₂CO₃ until the pH value rose above 8. After which, the aqueous solution was extracted with DCM (3×50 mL). The combined organic layers were rinsed with brine and dried over anhydrous Na₂SO₄. The solvent was removed using a rotary evaporator to give 11 (0.534 g, 90%) as a light green oil, which was used for the next step without further purification.

### (E)-N-(7-(ethylamino)-3H-phenoxazin-3-ylidene)ethanaminium (LGW-01-21)

Compound 5 (50 mg, 0.36 mmol) was dissolved in a solution of i-PrOH/H₂O (9/1, 1 mL) at 80° C. for 30 min. A suspended solution of 11 (69 mg, 0.38 mmol) and HClO₄ (70%, 35 μL) in 90% i-PrOH (2 mL) was added into the solution above in 4 portions over 1 h. The resulting solution was stirred overnight. During which time, the color of the reaction mixture changed from light brown to green and finally dark blue. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-01-21 (9 mg, 8%) as a dark blue solid.

LGW-01-23

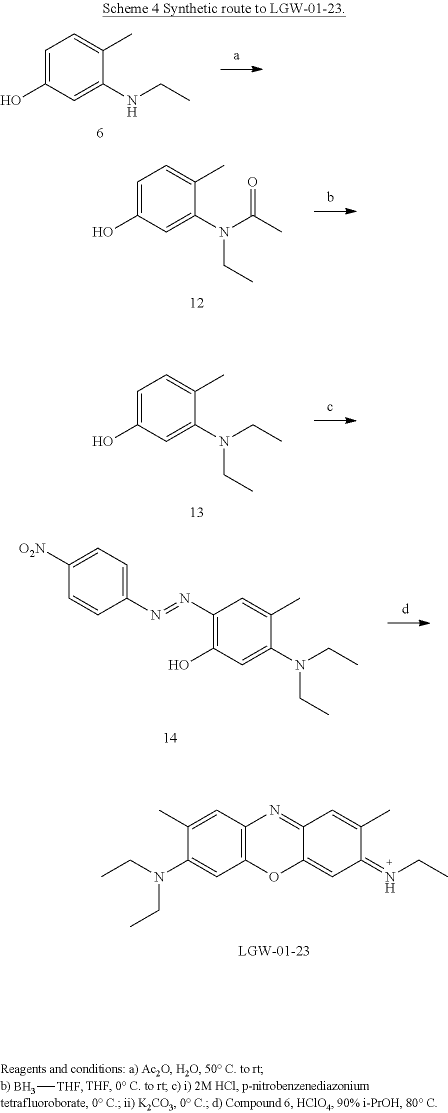

### N-ethyl-N-(5-hydroxy-2-methylphenyl)acetamide (12)

Compound 6 (1 g, 6.61 mmol) was suspended in 10 mL DI water, to which Acetic anhydride (1.9 mL, 19.84 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of DI water. The product was left in the funnel and air dried overnight to afford compound 12 (1.09 g, 85%) as a light gray solid, which was used for the next step without further purification.

### 3-(diethylamino)-4-methylphenol (13)

A solution of 12 (1 g, 5.17 mmol) in anhydrous THF (16 mL) was stirred in an ice bath under N₂ for 30 mins. Borane tetrahydrofuran complex solution (1 M, 16 mL) was added to the solution above using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, and the residue was purified by flash column chromatography with silica gel (25 g), using DCM/Hexane as eluent to give 13 (872 mg, 94%).

### (E)-5-(diethylamino)-4-methyl-2-((4-nitrophenyl)diazenyl)phenol (14)

Compound 13 (0.5 g, 2.79 mmol) was dissolved in 1 mL MeOH. The solution was chilled in an ice bath, then was treated with HCl (2 M, 10 mL). After 15 mins, p-nitrobenzenediazonium tetrafluoroborate (0.695 g, 2.93 mmol) was added to the solution above in 5 portions over 15 mins, then stirred at 0° C. for additional 1 h. During which time, the color of the reaction mixture changed from orange to dark red. After which, the solution was carefully basified with solid K₂CO₃ until pH value of the solution rose above 8. The deep red precipitate was collected via vacuum filtration and washed with small portions of DI water. The product was left in the funnel and air dried overnight to afford compound 14 (0.77 g, 84%), which was used for the next step without further purification.

### (Z)—N-(7-(diethylamino)-2,8-dimethyl-3H-phenoxazin-3-ylidene)ethanaminium (LGW-01-23)

Compound 6 (50 mg, 0.33 mmol) was dissolved in a solution of i-PrOH/H₂O (9/1, 3 mL) at 80° C. for 30 min. Compound 14 (109 mg, 0.33 mmol) was added to the solution above in 5 portions over 15 mins. Then the reaction mixture was treated with HClO₄ (70%, 29 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-01-23 (10.23 mg, 10%) as a dark blue solid.

LGW-01-25

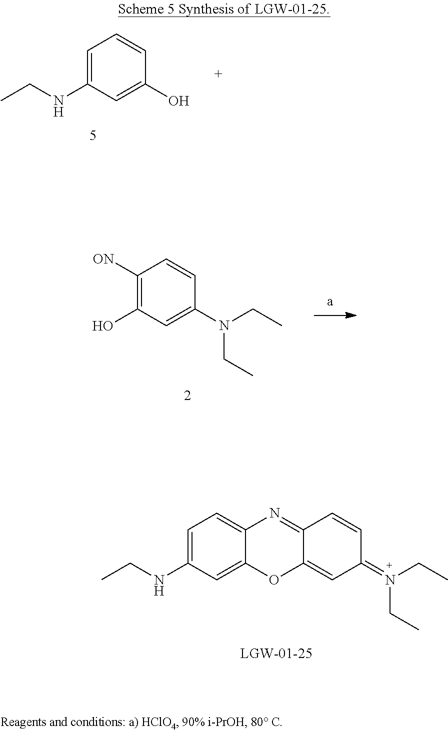

### N-ethyl-N-(7-(ethylamino)-3H-phenoxazin-3-ylidene)ethanaminium (LGW-01-25)

Compound 5 (50 mg, 0.36 mmol) was dissolved in a solution of i-PrOH/H₂O (9/1, 1 mL) at 80° C. for 30 min. A suspended solution of 2 (74 mg, 0.38 mmol) and HClO₄ (70%, 35 μL) in 90% i-PrOH (2 mL) was added into the solution above in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-01-25 (12 mg, 11%) as a dark blue solid.

LGW-01-39

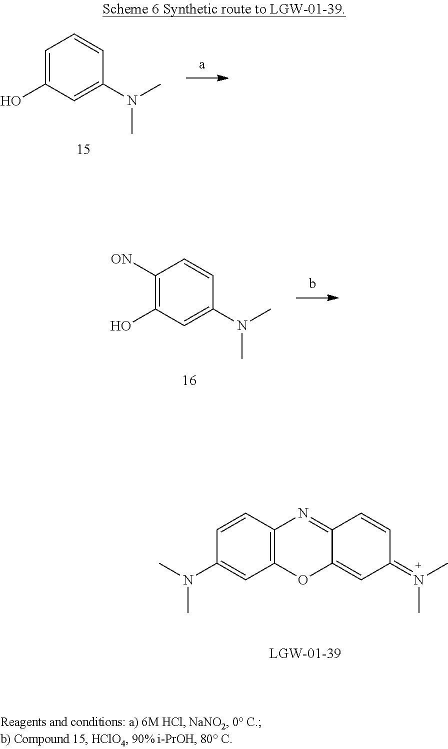

### 5-(dimethylamino)-2-nitrosophenol (16)

Compound 15 (1 g, 7.29 mmol) was dissolved in an ice-cold 6 M HCl solution (5 mL). To the solution above, NaNO₂ (0.513 g, 7.44 mmol) was added portion wise over 1 h while maintaining the temperature of the solution below 5 C, such that no brown NOx vapors were observed. The reaction mixture was stirred for additional 2 h. After which, the precipitate was filtered through a Büchner funnel and washed with small portions of ice-cold 2 M HCl solution. The product was left in the funnel and air dried overnight to afford compound 16 (1.01 g, 84%) as a yellow solid, which was used for the next step without further purification.

### N-(7-(dimethylamino)-3H-phenoxazin-3-ylidene)-N-methylmethanaminium (LGW-01-39)

Compound 15 (50 mg, 0.36 mmol) was dissolved in a solution of i-PrOH/H₂O (9/1, 1 mL) at 80° C. for 30 min. A suspended solution of 16 (64 mg, 0.38 mmol) and HClO₄ (70%, 35 μL) in 90% i-PrOH (2 mL) was added into the solution above in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-01-39 (67 mg, 69%) as a dark blue solid.

LGW-01-44

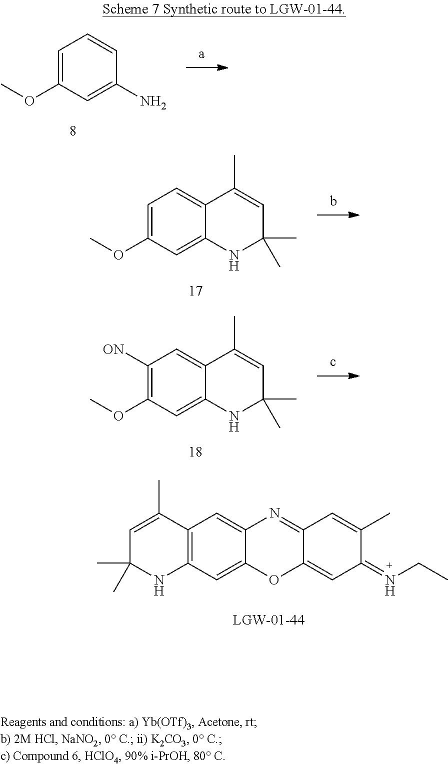

### 7-methoxy-2,2,4-trimethyl-1,2-dihydroquinoline (17)

Compound 17 was synthesized using a modified protocol published by Belov Vladimir et al (Chemistry—A European Journal 15, 10762-10776, doi:10.1002/chem.200901333 (2009)). Compound 8 (9.09 mL, 81.2 mmol) was diluted in acetone (200 mL) under N₂, to the solution above ytterbium(III) triflate (2.52 g, 4.06 mmol) was added. The resulting solution was stirred at rt for 24 h. After which, the reaction mixture was concentrated under reduced pressure. The residue was dissolved in EtOAc, which was washed with water, brine, and dried over anhydrous Na₂SO₄. The organic solvent was removed using rotary evaporator. The crude product was purified by flash column chromatography with silica gel (100 g), using EtOAc/Hexane as eluent to give compound 17 (13.50 g, 82%) as a pale yellow solid.

### 7-methoxy-2,2,4-trimethyl-6-nitroso-1,2-dihydroquinoline (18)

compound 17 (1 g, 4.92 mmol) was dissolved in an ice-cold 2 M HCl solution (15 mL). To the solution above, NaNO₂ (0.37 g, 5.41 mmol) was added portion wise over 1 h while maintaining the temperature of the solution below 5° C., such that no brown NOx vapors were observed. The reaction mixture was stirred for additional 2 h. The solution was carefully basified with solid K₂CO₃ until pH value of the above solution rose above 8. After which, the precipitate was filtered through a Buchner funnel and washed with small portions of DI water. The product was left in the funnel and air dried overnight to afford compound 18 (1.04 g, 91%) as a yellow-brownish solid, which was used for the next step without further purification.

### (Z)—N-(2,2,4,8-tetramethyl-1,2-dihydro-9H-pyrido[3,2-b]phenoxazin-9-ylidene)ethanaminium (LGW-01-44)

Compound 6 (50 mg, 0.33 mmol) was dissolved in a solution of i-PrOH/H₂O (9/1, 1 mL) at 80° C. for 30 min. A suspended solution of 18 (81 mg, 0.35 mmol) and HClO₄ (70%, 30 μL) in 90% i-PrOH (2 mL) was added into the solution above in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-01-44 (83 mg, 66%) as a dark blue solid.

LGW-01-56

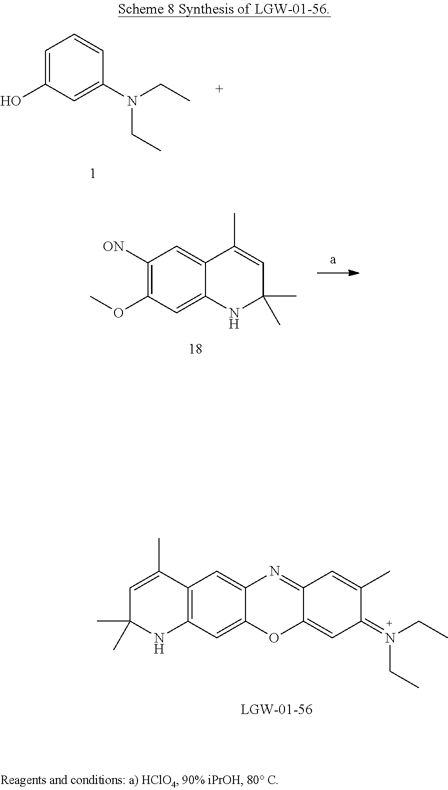

### N-ethyl-N-(2,2,4,8-tetramethyl-1,2-dihydro-9H-pyrido[3,2-b]phenoxazin-9-ylidene)ethanaminium (LGW-01-56)

Compound 1 (50 mg, 0.3 mmol) was dissolved in a solution of i-PrOH/H₂O (9/1, 1 mL) at 80° C. for 30 min. A suspended solution of 18 (74 mg, 0.32 mmol) and HClO₄ (70%, 30 μL) in 90% i-PrOH (2 mL) was added into the solution above in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-01-56 (69 mg, 58%) as a dark blue solid.

LGW-01-61

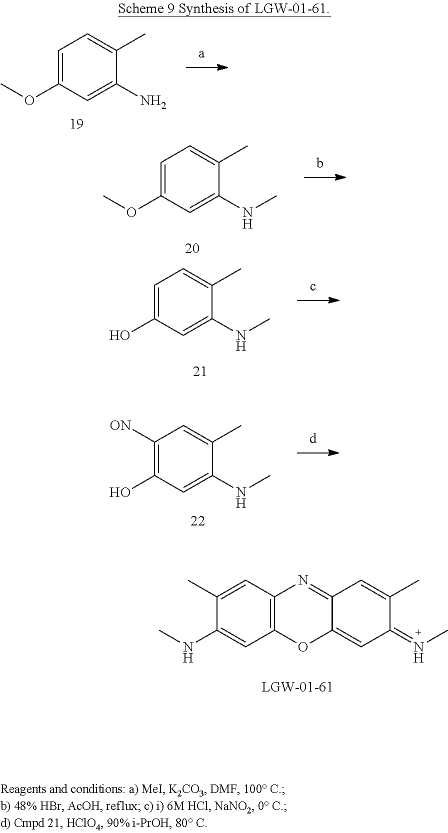

### 5-methoxy-N,2-dimethylaniline (20)

To a suspension of compound 19 (2 g, 14.58 mmol) and K₂CO₃ (2.12 g, 15.31 mmol) in anhydrous DMF (10 mL) under N₂, was added Mel (0.91 mL, 14.58 mmol) at rt. The reaction mixture was then heated up to 100° C. and stirred for additional 2 h. The solution was cooled down to rt and concentrated under reduced pressure. The crude product was diluted with DI water, and the resulting suspension was extracted with DCM (3×50 mL). The combined organic layers were rinsed with brine, dried over anhydrous Na₂SO₄. The solvent was removed using a rotary evaporator, and the residue was purified by flash column chromatography with silica gel (50 g), using EtOAc/Hexane as eluent to give compound 20 (1.41 g, 64%).

### 4-methyl-3-(methylamino)phenol (21)

Compound 20 (0.9 g, 5.95 mmol) was dissolved in glacial AcOH (9 mL) at rt after which aqueous HBr (48%, 9 mL) was added. The resulting solution was heated at 110° C. for 5 h before cooling. After which, the reaction mixture was diluted with 50 mL DI water, and the pH of the solution was adjusted to 5-6 with 2 M NaOH solution. The aqueous solution was extracted with DCM (3×50 mL). The combined organic layers were rinsed with brine, dried over anhydrous Na₂SO₄. The solvent was removed using a rotary evaporator and the residue was purified by flash column chromatography with silica gel (30 g), using EtOAc/Hexane as eluent to give compound 21 (478 mg, 59%).

### 4-methyl-5-(methylamino)-2-nitrosophenol (22)

Compound 21 (200 mg, 1.46 mmol) was dissolved in an ice-cold 6 M HCl solution (2 mL). To the solution above, NaNO₂ (106 mg, 1.53 mmol) was added portion wise over 1 h while maintaining the temperature of the solution below 5° C., such that no brown NOx vapors were observed. The reaction mixture was stirred for additional 2 h. After which, the precipitate was filtered through a Büchner funnel and washed with small portions of ice-cold 2 M HCl solution. The product was left in the funnel and air dried overnight to afford compound 22 (192 mg, 79%), which was used for the next step without further purification.

### (Z)—N-(2,8-dimethyl-7-(methylamino)-3H-phenoxazin-3-ylidene)methanaminium (LGW-01-61)

Compound 21 (40 mg, 0.29 mmol) was dissolved in a solution of i-PrOH/H₂O (9/1, 1 mL) at 80° C. for 30 min. A suspended solution of 22 (51 mg, 0.31 mmol) and HClO₄ (70%, 30 μL) in 90% i-PrOH (2 mL) was added to the solution in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-01-61 (58 mg, 64%) as a dark blue solid.

LGW-01-64

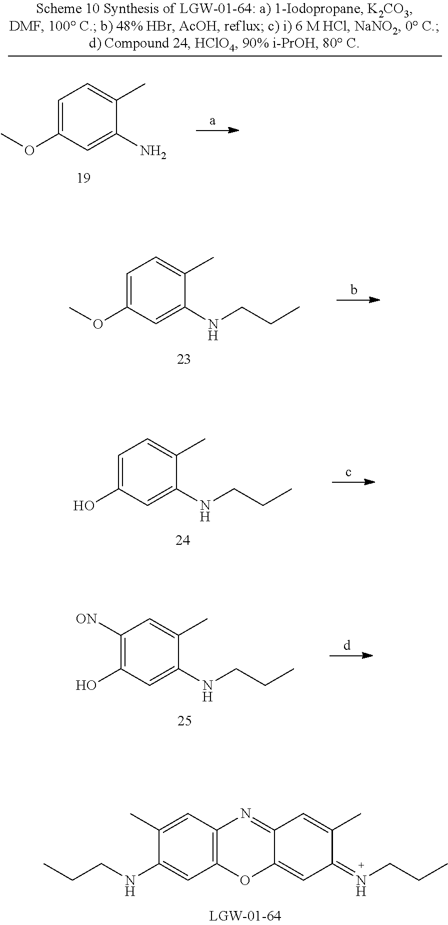

### 5-methoxy-2-methyl-N-propylaniline (23)

To a suspension of compound 19 (1 g, 5.58 mmol) and K₂CO₃ (2.12 g, 15.31 mmol) in anhydrous DMF (10 mL) under N₂, was added 1-Iodopropane (1.42 mL, 14.58 mmol) at rt. The reaction mixture was heated to 100° C., and stirred for additional 2 h. The solution was cooled down to rt and concentrated under reduced pressure. The crude product was diluted with DI water, and the resulting suspension was extracted with DCM (3×50 mL). The combined organic layers were rinsed with brine and dried over anhydrous Na₂SO₄. The solvent was removed using a rotary evaporator, and the residue was purified by flash column chromatography with silica gel (50 g), using EtOAc/Hexane as eluent to give compound 23 (1.53 g, 59%).

### 4-methyl-3-(propylamino)phenol (24)

Compound 23 (1 g, 5.58 mmol) was dissolved in glacial AcOH (9 mL) at rt, aqueous HBr (48%, 9 mL) was added to the solution above. The resulting solution was heated at 110° C. for 5 h before cooling. After which the reaction mixture was diluted with 50 mL DI water, and the pH of the solution was adjusted to 5-6 with 2 M NaOH solution. The aqueous solution was extracted with DCM (3×50 mL). The combined organic layers were rinsed with brine, dried over anhydrous Na₂SO₄. The solvent was removed using a rotary evaporator and the residue was purified by flash column chromatography with silica gel (30 g), using EtOAc/Hexane as eluent to give compound 24 (518 mg, 56%) as brown oil.

### 4-methyl-2-nitroso-5-(propylamino)phenol (25)

Compound 24 (200 mg, 1.21 mmol) was dissolved in an ice-cold 6 M HCl solution (2 mL). To the solution above, NaNO₂ (88 mg, 1.27 mmol) was added portion wise over 1 h while maintaining the temperature of the solution below 5° C., such that no brown NOx vapors were observed. The reaction mixture was stirred for additional 2 h. After which, the precipitate was filtered through a Büchner funnel and washed with small portions of ice-cold 2 M HCl solution. The product was left in the funnel and air dried overnight to afford compound 25 (215 mg, 91%) as an orange solid, which was used for the next step without further purification.

### (Z)—N-(2,8-dimethyl-7-(propylamino)-3H-phenoxazin-3-ylidene)propan-1-aminium (LGW-01-64)

Compound 24 (50 mg, 0.3 mmol) was dissolved in a solution of i-PrOH/H₂O (9/1, 1 mL) at 80° C. for 30 min. A suspended solution of 25 (62 mg, 0.31 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-01-64 (72 mg, 65%) as a dark blue solid.

LGW-01-99

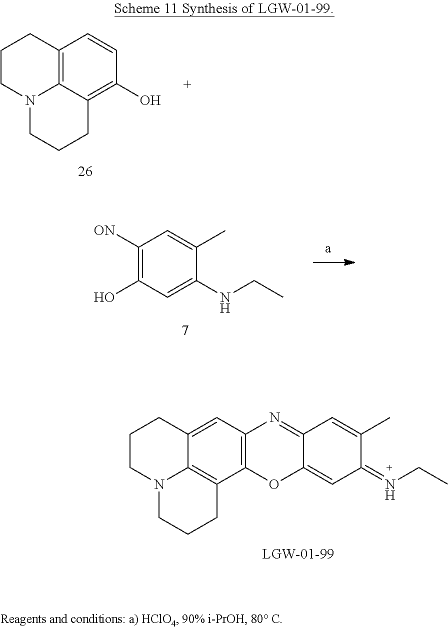

### (Z)—N-(11-methyl-2,3,6,7-tetrahydro-1H,5H,12H-quinolizino[1,9-bc]phenoxazin-12-ylidene)ethanaminium (LGW-01-99)

Compound 26 (50 mg, 0.26 mmol) was dissolved in a solution of i-PrOH/H₂O (9/1, 1 mL) at 80° C. for 30 min. A suspended solution of 7 (50 mg, 0.27 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-01-99 (33 mg, 33%) as a dark blue solid.

LGW-02-57

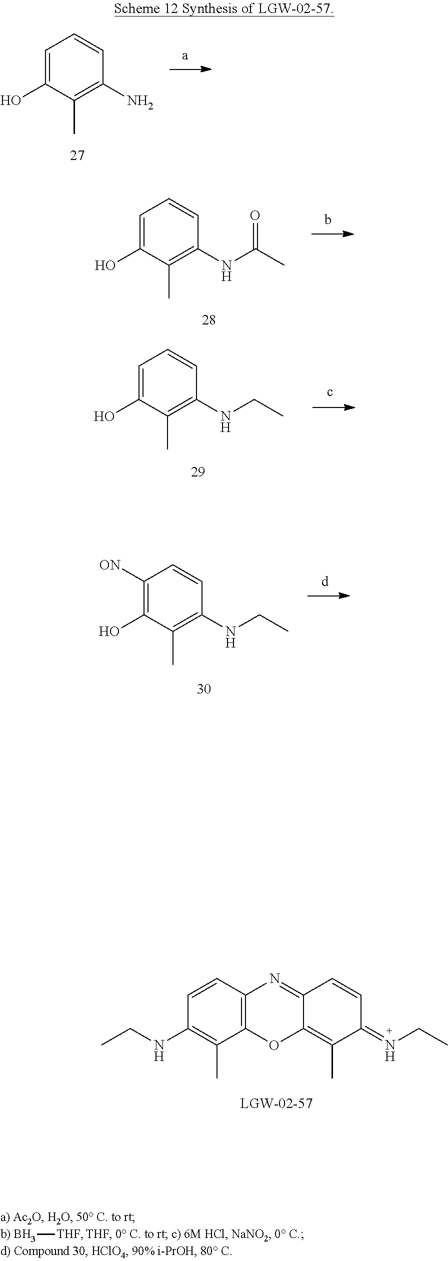

### N-(3-hydroxy-2-methylphenyl)acetamide (28)

Compound 27 (2 g, 16.24 mmol) was suspended in 10 mL DI water, to which Acetic anhydride (4.61 mL, 48.72 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of DI water. The product was left in the funnel and air dried overnight to afford compound 28 (1.65 g, 62%) as a light gray solid, which was used for the next step without further purification.

### 3-(ethylamino)-2-methylphenol (29)

A solution of 28 (1.5 g, 9.08 mmol) in anhydrous THF (25 mL) was stirred in an ice bath under N₂ for 30 mins. Borane tetrahydrofuran complex solution (1 M, 27 mL) was added to the solution above using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, and the residue was purified by flash column chromatography with silica gel (25 g), using DCM/Hexane as eluent to obtain 29 (1.18 g, 86%) as brown oil.

### 3-(ethylamino)-2-methyl-6-nitrosophenol (30)

Compound 29 (300 mg, 1.98 mmol) was dissolved in an ice-cold 6 M HCl solution (2 mL). To the solution above, NaNO₂ (144 mg, 2.08 mmol) was added portion wise over 1 h while maintaining the temperature of the solution below 5° C., such that no brown NOx vapors were observed. The reaction mixture was stirred for additional 2 h. After which, the precipitate was filtered through a Büchner funnel and washed with small portions of ice-cold 2 M HCl solution. The product was left in the funnel and air dried overnight to afford compound 30 (257 mg, 72%) as a brown solid, which was used for the next step without further purification.

### (E)-N-(7-(ethylamino)-4,6-dimethyl-3H-phenoxazin-3-ylidene)ethanaminium (LGW-02-57)

Compound 29 (50 mg, 0.33 mmol) was dissolved in a solution of i-PrOH/H₂O (9/1, 1 mL) at 80° C. for 30 min. A suspended solution of 30 (63 mg, 0.35 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-02-57 (8 mg, 7%) as a dark blue solid.

LGW-02-58

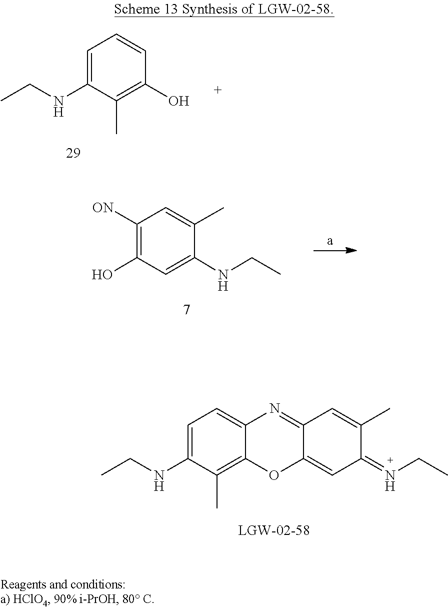

### (Z)—N-(7-(ethylamino)-2,6-dimethyl-3H-phenoxazin-3-ylidene)ethanaminium (LGW-02-58)

Compound 29 (50 mg, 0.33 mmol) was dissolved in a solution of i-PrOH/H₂O (9/1, 1 mL) at 80° C. for 30 min. A suspended solution of 7 (63 mg, 0.35 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-02-58 (72 mg, 73%) as a dark blue solid.

LGW-02-59

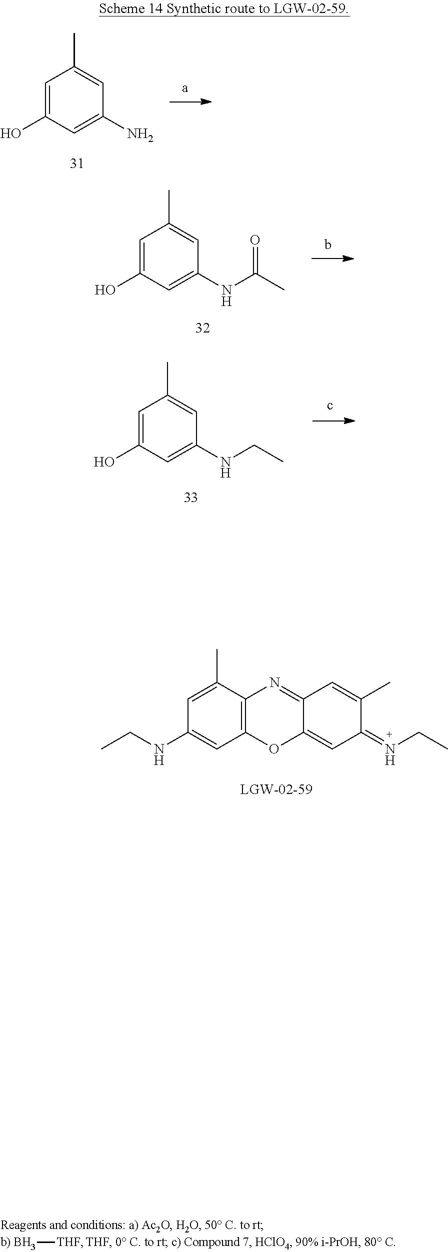

### N-(3-hydroxy-5-methylphenyl)acetamide (32)

Compound 31 (1 g, 8.12 mmol) was suspended in 10 mL DI water, to which Acetic anhydride (4.61 mL, 48.72 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of DI water. The product was left in the funnel and air dried overnight to afford compound 32 (1.28 g, 95%) as a light gray solid, which was used for the next step without further purification.

### 3-(ethylamino)-5-methylphenol (33)

A solution of 32 (1.2 g, 7.26 mmol) in anhydrous THF (22 mL) was stirred in an ice bath under N₂ for 30 mins. Borane tetrahydrofuran complex solution (1 M, 22 mL) was added to the solution above using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, the residue was purified by flash column chromatography with silica gel (25 g), using DCM/Hexane as eluent to give 33 (1.04 g, 94%).

### (Z)—N-(7-(ethylamino)-2,9-dimethyl-3H-phenoxazin-3-ylidene)ethanaminium (LGW-02-59)

Compound 33 (50 mg, 0.33 mmol) was dissolved in a solution of i-PrOH/H₂O (9/1, 1 mL) at 80° C. for 30 min. A suspended solution of 7 (63 mg, 0.35 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-02-59 (9 mg, 8%) as a dark blue solid.

LGW-02-60

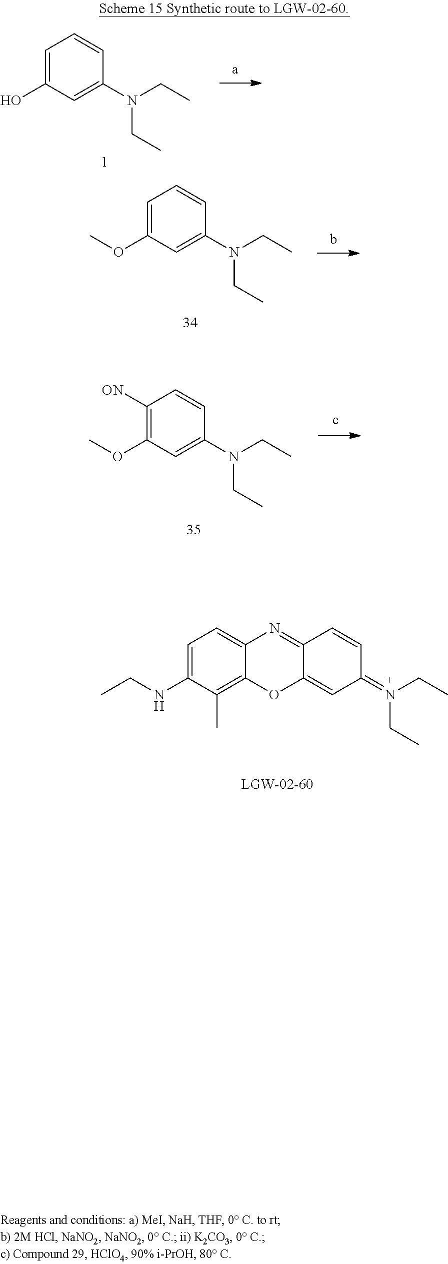

### N,N-diethyl-3-methoxyaniline (34)

Compound 1 (5 g, 30.26 mmol) was dissolved in anhydrous THF (50 mL) under N₂, and chilled in an ice bath for 30 mins. NaH (60%, 3.63 g, 90.78 mmol) was added to the solution in 3 portions over 10 mins while the temperature was maintained below 5° C. After 10 mins, Mel (7.54 mL, 121 mmol) was added into the reaction mixture in one portion. The resulting suspension was slowly warmed to rt and stirred overnight. Upon completion of the reaction, DI water was added to the reaction mixture to destroy excess NaH. Organic solvent was removed under reduced pressure, and the residue was extracted with DCM (3×100 mL). The combined organic layers were rinsed with brine and dried over anhydrous Na₂SO₄. The solvent was removed by rotary evaporation and the residue purified by flash column chromatography with silica gel (100 g), using DCM/Hexane as eluent to give compound 34 (4.70 g, 88%) as clear oil.

### N,N-diethyl-3-methoxy-4-nitrosoaniline (35)

Compound 34 (1.08 g, 6.02 mmol) was dissolved in an ice-cold 2 M HCl solution (15 mL). To the solution above, NaNO₂ (0.457 g, 6.63 mmol) was added portion wise over 1 h while maintaining the temperature of the solution below 5° C., such that no brown NOx vapors were observed. The reaction mixture was stirred for an additional 2 h. The solution was carefully basified with solid K₂CO₃ until pH value of the solution rose above 8. After which, the precipitate was filtered through a Buchner funnel and washed with small portions of DI water. The product was left in the funnel and air dried overnight to afford compound 35 (1.05 g, 84%) as a green solid, which was used for the next step without further purification.

### N-ethyl-N-(7-(ethylamino)-6-methyl-3H-phenoxazin-3-ylidene)ethanaminium (LGW-02-60)

Compound 29 (50 mg, 0.33 mmol) was dissolved in a solution of i-PrOH/H₂O (9/1, 1 mL) at 80° C. for 30 min. A suspended solution of 35 (72 mg, 0.35 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-02-60 (95 mg, 81%) as a dark blue solid.

LGW-02-61

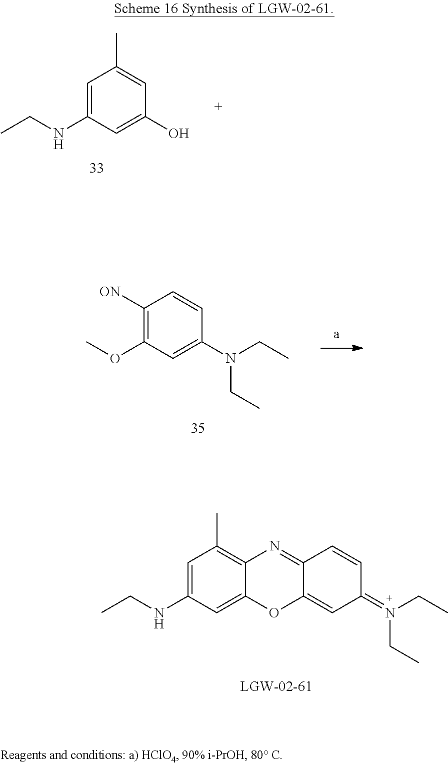

### N-ethyl-N-(7-(ethylamino)-9-methyl-3H-phenoxazin-3-ylidene)ethanaminium (LGW-02-61)

Compound 33 (50 mg, 0.33 mmol) was dissolved in a solution of i-PrOH/H₂O (9/1, 1 mL) at 80° C. for 30 min. A suspended solution of 35 (72 mg, 0.35 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-02-61 (52 mg, 44%) as a dark blue solid.

LGW-02-86

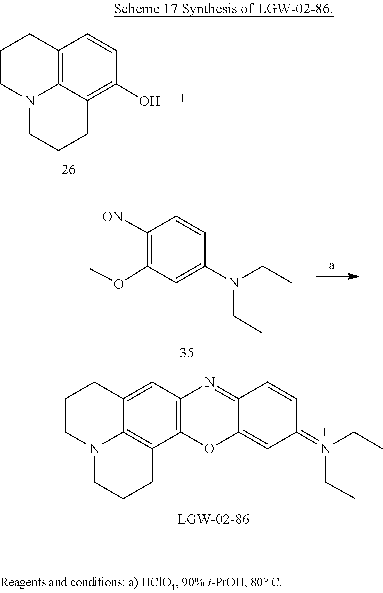

### N-ethyl-N-(2,3,6,7-tetrahydro-1H,5H,12H-quinolizino[1,9-bc]phenoxazin-12-ylidene)ethanaminium (LGW-02-86)

Compound 26 (50 mg, 0.26 mmol) was dissolved in a solution of i-PrOH/H₂O (9/1, 1 mL) at 80° C. for 30 min. A suspended solution of 35 (58 mg, 0.28 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-02-86 (36 mg, 34%) as a dark blue solid.

LGW-02-87

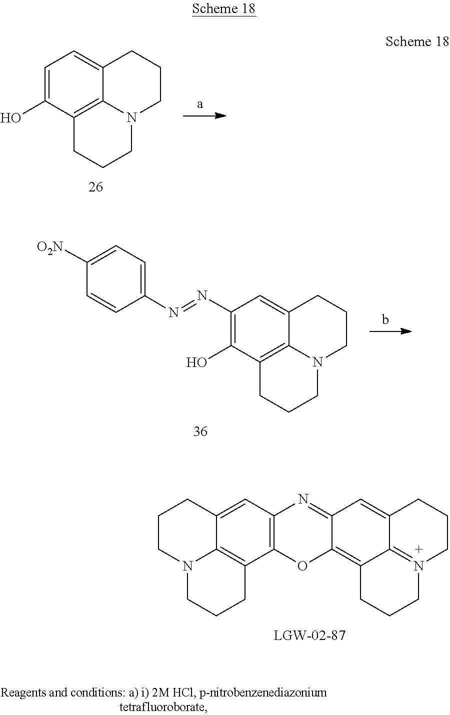

0° C.; ii) K₂CO₃, 0° C.; b) Compound 26, HClO₄, 90% i-PrOH, 80° C.

### (E)-9-((4-nitrophenyl)diazenyl)-2,3,6,7-tetrahydro-1H,5H-pyrido[3,2,1-ij]quinolin-8-ol (36)

Compound 26 (0.13 g, 0.69 mmol) was dissolved in 1 mL MeOH. The solution was chilled in an ice bath, then was treated with HCl (2 M, 10 mL). After 15 mins, p-nitrobenzenediazonium tetrafluoroborate (0.18 g, 0.76 mmol) was added to the solution in 5 portions over 15 mins, then stirred at 0° C. for additional 1 h. During which time, the color of the reaction mixture changed from orange to dark red. After which, the solution was carefully basified with solid K₂CO₃ until pH value of the solution rose above 8. The deep red precipitate was collected via vacuum filtration and washed with small portions of DI water. The product was left in the funnel and air dried overnight to afford compound 36 (186 mg, 80%) as a dark red solid, which was used for the next step without further purification.

### 2,3,6,7,12,13,16,17-octahydro-1H,5H,11H,15H-diquinolizino[1,9-bc:1′,9′-hi]phenoxazine-4-ium (LGW-02-87)

Compound 26 (50 mg, 0.26 mmol) was dissolved in a solution of i-PrOH/H₂O (9/1, 3 mL) at 80° C. for 30 min. Compound 36 (89 mg, 0.33 mmol) was added to the solution above in 5 portions over 15 mins. Then the reaction mixture was treated with HClO₄ (70%, 25 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-02-87 (37 mg, 34%) as a dark blue solid.

LGW-02-91

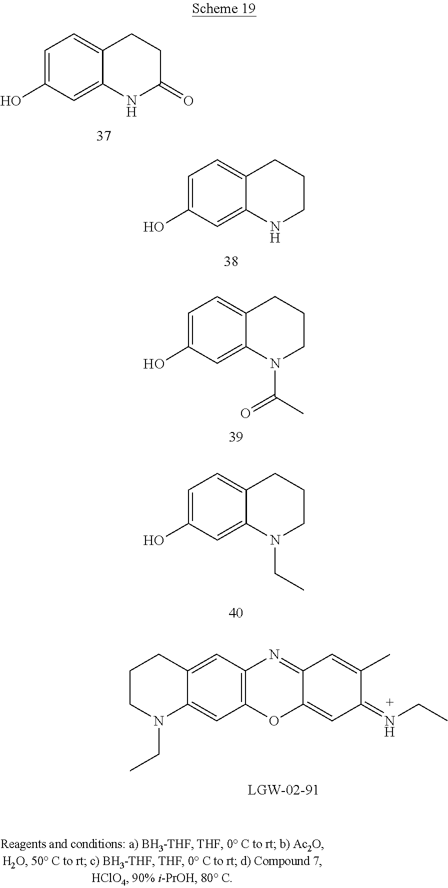 [SMILES: O=C1CCc2ccc(O)cc2N1 ; Oc1ccc2c(c1)NCCC2 ; CC(=O)N1CCCc2ccc(O)cc21 ; CCN1CCCc2ccc(O)cc21 ; CC/N=c1/cc2oc3cc4c(cc3nc-2cc1C)CCCN4CC]

### 1,2,3,4-tetrahydroquinolin-7-ol (38)

A solution of 37 (2 g, 12.87 mmol) in anhydrous THF (38 mL) was stirred in an ice bath under N₂ for 30 mins. Borane tetrahydrofuran complex solution (1 M, 38 mL) was added to the solution above using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, the residue was purified by flash column chromatography with silica gel (50 g), using EtOAc/Hexane as eluent to give 38 (1.83 g, 95%) as a yellow solid.

### 1-(7-hydroxy-3,4-dihydroquinolin-1(2H)-yl)ethan-1-one (39)

Compound 38 (1 g, 6.7 mmol) was suspended in 10 mL DI water, to which Acetic anhydride (1.9 mL, 20.11 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of DI water. The product was left in the funnel and air dried overnight to afford compound 39 (1.13 g, 88%) as a white solid, which was used for the next step without further purification.

### 1-ethyl-1,2,3,4-tetrahydroquinolin-7-ol (40)

A solution of 39 (1.13 g, 5.91 mmol) in anhydrous THF (18 mL) was stirred in an ice bath under N₂ for 30 mins. Borane tetrahydrofuran complex solution (1 M, 18 mL) was added to the solution above using a syringe pump over 30 mins, while maintaining the temperature of the solution below 5° C. The resulting reaction mixture was left in the ice bath and slowly warmed to rt. After 24 h, the solution was placed in an ice bath again, and excess borane reagent was destroyed by carefully adding MeOH until no gas evolved. The solvent was evaporated under reduced pressure, the residue was purified by flash column chromatography with silica gel (25 g), using EtOAc/Hexane as eluent to obtain 40 (0.96 g, 92%) as dark oil.

### (Z)—N-(1-ethyl-8-methyl-1,2,3,4-tetrahydro-9H-pyrido[3,2-b]phenoxazin-9-ylidene)ethanaminium (LGW-02-91)

Compound 40 (50 mg, 0.28 mmol) was dissolved in a solution of i-PrOH/H₂O (9/1, 1 mL) at 80° C. for 30 min. A suspended solution of 35 (54 mg, 0.30 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-02-91 (39 mg, 38%) as a dark blue solid.

LGW-02-92

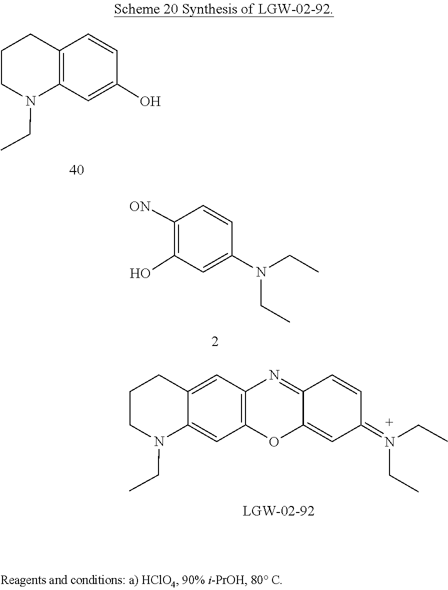

### N-ethyl-N-(1-ethyl-1,2,3,4-tetrahydro-9H-pyrido[3,2-b]phenoxazin-9-ylidene)ethanaminium (LGW-02-92)

Compound 40 (50 mg, 0.28 mmol) was dissolved in a solution of i-PrOH/H₂O (9/1, 1 mL) at 80° C. for 30 min. A suspended solution of 2 (58 mg, 0.30 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-02-92 (43 mg, 40%) as a dark blue solid.

LGW-02-95

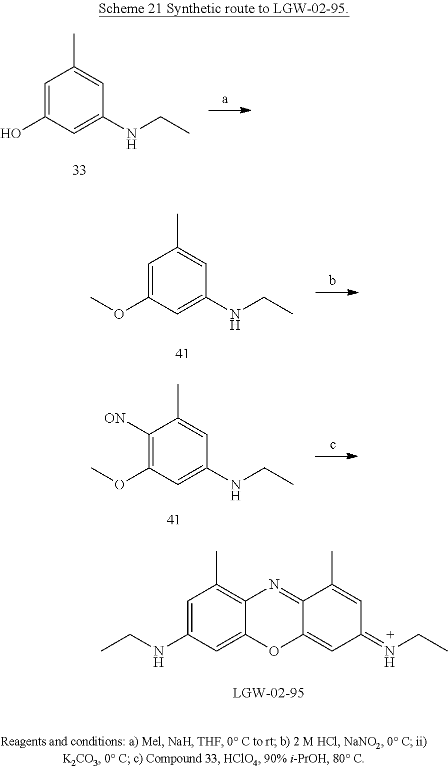

### N-ethyl-3-methoxy-5-methylaniline (41)

Compound 33 (0.5 g, 3.31 mmol) was dissolved in anhydrous THF (10 mL) under N₂, and chilled in an ice bath for 30 mins. NaH (60%, 139 mg, 3.47 mmol) was added to the solution in 3 portions over 10 mins while the temperature was maintained below 5° C. After 10 mins, Mel (0.216 mL, 3.47 mmol) was added into the reaction mixture in one portion. The resulting suspension was slowly warmed up to rt and stirred overnight. Upon completion of the reaction, DI water was added to the reaction mixture to destroy excess NaH. Organic solvent was removed under reduced pressure, and the residue was extracted with DCM (3×25 mL). The combined organic layers were rinsed with brine and dried over anhydrous Na₂SO₄. The solvent was removed using a rotary evaporator and the residue was purified by flash column chromatography with silica gel (100 g), using DCM/Hexane as eluent to give compound 41 (0.37 g, 68%).

### N-ethyl-3-methoxy-5-methyl-4-nitrosoaniline (42)

Compound 41 (200 mg, 1.21 mmol) was dissolved in an ice-cold 2 M HCl solution (5 mL). NaNO₂ (92 mg, 1.33 mmol) was added portion wise over 1 h while maintaining the temperature of the solution below 5° C., such that no brown NOx vapors were observed. The reaction mixture was stirred for additional 2 h. The solution was carefully basified with solid K₂CO₃ until pH value of the above solution rose above 8. After which, the aqueous solution was extracted with DCM (3×25 mL). The combined organic layers were rinsed with brine and dried over anhydrous Na₂SO₄. The solvent was removed using a rotary evaporator to give 42 (109 mg, 46%) as a light green oil, which was used for the next step without further purification.

### (E)-N-(7-(ethylamino)-1,9-dimethyl-3H-phenoxazin-3-ylidene)ethanaminium (LGW-02-95)

Compound 33 (50 mg, 0.33 mmol) was dissolved in a solution of i-PrOH/H₂O (9/1, 1 mL) at 80° C. for 30 min. A suspended solution of 42 (68 mg, 0.35 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-02-95 (9 mg, 7%) as a dark blue solid.

LGW-02-99

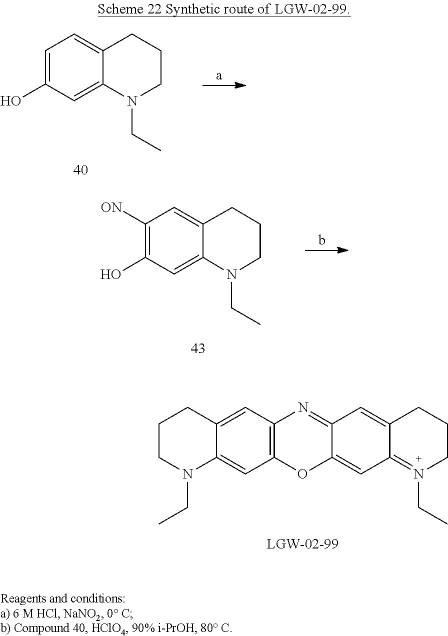

### 1-ethyl-6-nitroso-1,2,3,4-tetrahydroquinolin-7-ol (43)

Compound 40 (450 mg, 2.54 mmol) was dissolved in an ice-cold 6 M HCl solution (8 mL). NaNO₂ (184 mg, 2.67 mmol) was added portion wise over 1 h while maintaining the temperature of the solution below 5° C., such that no brown NOx vapors were observed. The reaction mixture was stirred for additional 2 h. After which, the precipitate was filtered through a Büchner funnel and washed with small portions of ice-cold 2 M HCl solution. The product was left in the funnel and air dried overnight to afford compound 43 (392 mg, 75%), which was used for the next step without further purification.

### 1,11-diethyl-3,4,8,9,10,11-hexahydro-2H-dipyrido[3,2-b:2′,3′-i]phenoxazin-1-ium (LGW-02-99)

Compound 40 (33 mg, 0.19 mmol) was dissolved in a solution of i-PrOH/H₂O (9/1, 1 mL) at 80° C. for 30 min. A suspended solution of 43 (40 mg, 0.2 mmol) and HClO₄ (70%, 20 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-02-99 (12 mg, 19%) as a dark blue solid.

LGW-03-01

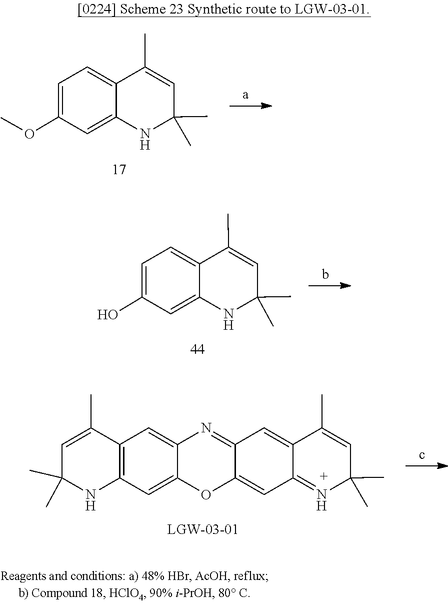

### 2,2,4-trimethyl-1,2-dihydroquinolin-7-ol (44)

Compound 17 (1 g, 4.92 mmol) was dissolved in glacial AcOH (4 mL) at rt and aqueous HBr (48%, 4 mL) was added. The resulting solution was heated at 110° C. for 5 h before cooling. After which the reaction mixture was diluted with 50 mL DI water, and the pH of the solution was adjusted to 5-6 with 2 M NaOH solution. The aqueous solution was extracted with DCM (3×50 mL). The combined organic layers were rinsed with brine and dried over anhydrous Na₂SO₄. The solvent was removed using a rotary evaporator and the residue was purified by flash column chromatography with silica gel (30 g), using EtOAc/Hexane as eluent to give compound 44 (619 mg, 67%) as a brown solid.

### 2,2,4,8,10,10-hexamethyl-10,11-dihydro-2H-dipyrido[3,2-b:2′,3′-i]phenoxazin-1-ium (LGW-03-01)

Compound 44 (55 mg, 0.29 mmol) was dissolved in a solution of i-PrOH/H₂O (9/1, 1 mL) at 80° C. for 30 min. A suspended solution of 18 (71 mg, 0.31 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-01 (17 mg, 16%) as a dark blue solid.

LGW-03-06

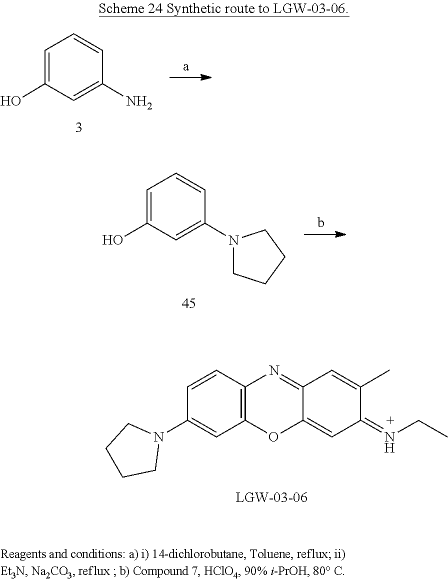 [SMILES: Nc1cccc(O)c1 ; Oc1cccc(N2CCCC2)c1 ; CC/N=c1/cc2oc3cc(N4CCCC4)ccc3nc-2cc1C]

### 3-(pyrrolidin-1-yl)phenol (45)

To a suspension of compound 3 (5 g, 45.82 mmol) in anhydrous toluene, was added 1,4-dichlorobutane (5.52 mL, 50.40 mmol). The reaction mixture was refluxed for 24 h then cooled down to rt. After which, Et₃N (9.58 mL, 68.73 mmol) and Na₂CO₃ (4.86 g, 45.82 mmol) in 10 mL DI water was added to the reaction flask. The resulting reaction mixture was refluxed for an additional 24 h. Upon completion of the reaction, organic solvent was removed under reduced pressure. The aqueous phase was extracted with DCM (3×100 mL). The combined organic layers were rinsed with brine and dried over anhydrous Na₂SO₄. The solvent was removed using a rotary evaporator and the residue was purified by flash column chromatography with silica gel (100 g), using DCM/Hexane as eluent to give compound 45 (5.37 g, 72%) as a light gray solid.

### (Z)—N-(2-methyl-7-(pyrrolidin-1-yl)-3H-phenoxazin-3-ylidene)ethanaminium (LGW-03-06)

Compound 45 (50 mg, 0.31 mmol) was dissolved in a solution of i-PrOH/H₂O (9/1, 1 mL) at 80° C. for 30 min. A suspended solution of 7 (58 mg, 0.32 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-06 (64 mg, 59%) as a dark blue solid.

LGW-03-07

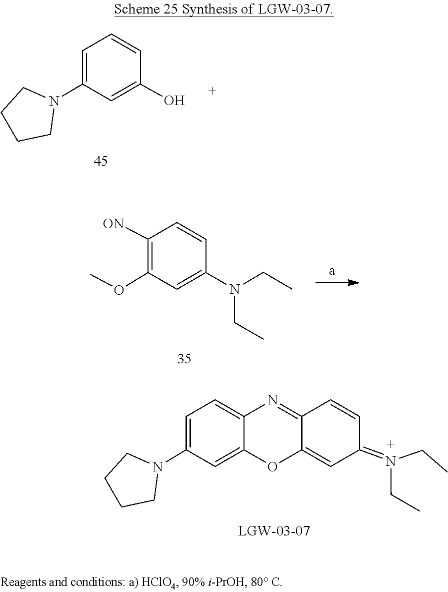

### N-ethyl-N-(7-(pyrrolidin-1-yl)-3H-phenoxazin-3-ylidene)ethanaminium (LGW-03-07)

Compound 45 (50 mg, 0.31 mmol) was dissolved in a solution of i-PrOH/H₂O (9/1, 1 mL) at 80° C. for 30 min. A suspended solution of 35 (67 mg, 0.32 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-07 (13 mg, 12%) as a dark blue solid.

LGW-03-12

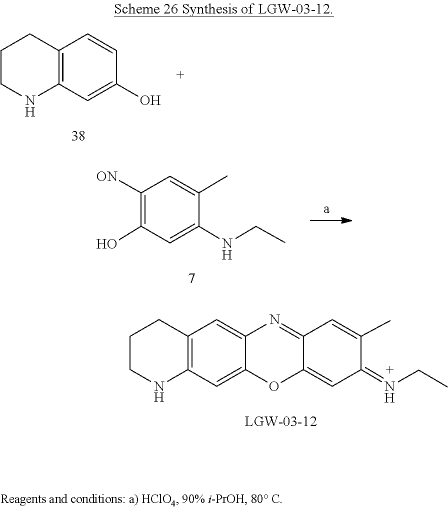

### (Z)—N-(8-methyl-1,2,3,4-tetrahydro-9H-pyrido[3,2-b]phenoxazin-9-ylidene)ethanaminium (LGW-03-12)

Compound 38 (50 mg, 0.34 mmol) was dissolved in a solution of i-PrOH/H₂O (9/1, 1 mL) at 80° C. for 30 min. A suspended solution of 7 (63 mg, 0.35 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-12 (57 mg, 50%) as a dark blue solid.

LGW-03-13

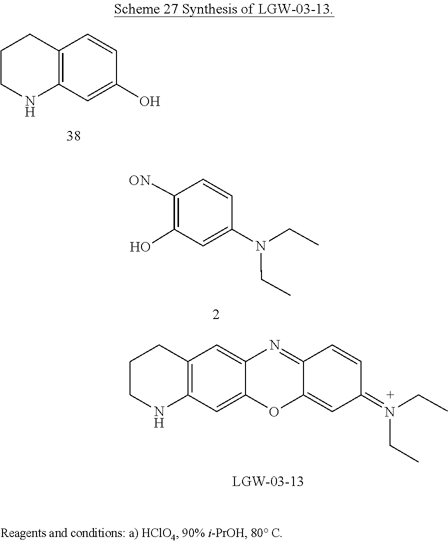

### N-ethyl-N-(1,2,3,4-tetrahydro-9H-pyrido[3,2-b]phenoxazin-9-ylidene)ethanaminium (LGW-03-13)

Compound 38 (50 mg, 0.34 mmol) was dissolved in a solution of i-PrOH/H₂O (9/1, 1 mL) at 80° C. for 30 min. A suspended solution of 2 (68 mg, 0.35 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-13 (23 mg, 20%) as a dark blue solid.

LGW-03-18

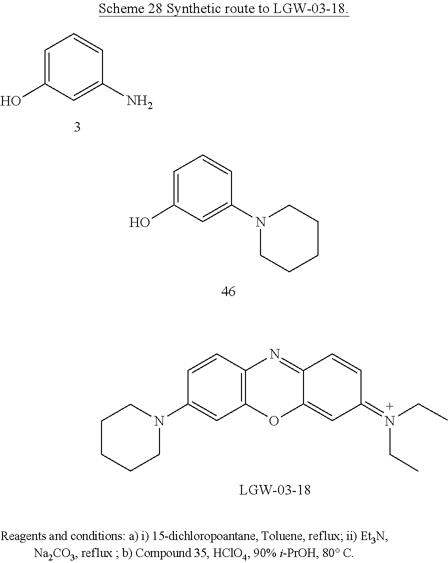

### 3-(piperidin-1-yl)phenol (46)

To a suspension of compound 3 (5 g, 45.82 mmol) in anhydrous toluene, was added 1,4-dichloropentane (6.64 mL, 50.40 mmol). The reaction mixture was refluxed for 24 h then cooled down to rt. After which, Et₃N (9.58 mL, 68.73 mmol) and Na₂CO₃ (4.86 g, 45.82 mmol) in 10 mL DI water was added to the reaction flask. The resulting reaction mixture was refluxed for an additional 24 h. Upon completion of the reaction, organic solvent was removed under reduced pressure. The aqueous phase was extracted with DCM (3×100 mL). The combined organic layers were rinsed with brine and dried over anhydrous Na₂SO₄. The solvent was removed using a rotary evaporator and the residue was purified by flash column chromatography with silica gel (100 g), using DCM/Hexane as eluent to give compound 46 (5.71 g, 70%).

### N-ethyl-N-(7-(piperidin-1-yl)-3H-phenoxazin-3-ylidene)ethanaminium (LGW-03-18)

Compound 46 (50 mg, 0.28 mmol) was dissolved in a solution of i-PrOH/H₂O (9/1, 1 mL) at 80° C. for 30 min. A suspended solution of 35 (62 mg, 0.30 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-18 (49 mg, 46%) as a dark blue solid.

LGW-03-21

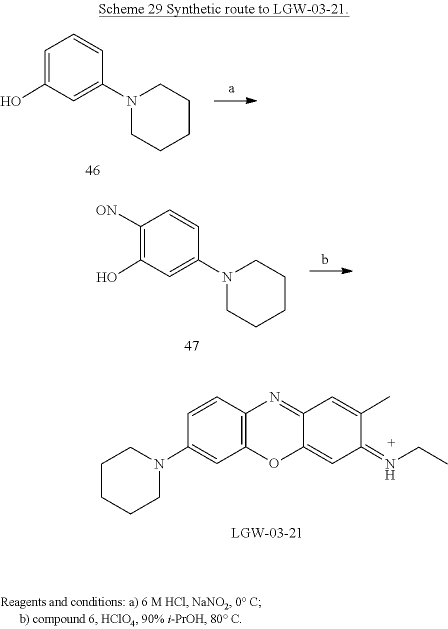

### 2-nitroso-5-(piperidin-1-yl)phenol (47)

Compound 46 (0.5 g, 2.82 mmol) was dissolved in an ice-cold 6 M HCl solution (5 mL). To the solution, NaNO₂ (0.214 g, 3.10 mmol) was added portion wise over 1 h while maintaining the temperature of the solution below 5° C., such that no brown NOx vapors were observed. The reaction mixture was stirred for additional 2 h. After which, the precipitate was filtered through a Büchner funnel and washed with small portions of ice-cold 2 M HCl solution. The product was left in the funnel and air dried overnight to afford compound 47 (0.514 g, 88%) as a brown solid, which was used for the next step without further purification.

### (Z)—N-(2-methyl-7-(piperidin-1-yl)-3H-phenoxazin-3-ylidene)ethanaminium (LGW-03-21)

Compound 6 (30 mg, 0.2 mmol) was dissolved in a solution of i-PrOH/H₂O (9/1, 1 mL) at 80° C. for 30 min. A suspended solution of 47 (43 mg, 0.35 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-21 (12 mg, 17%) as a dark blue solid.

LGW-03-23

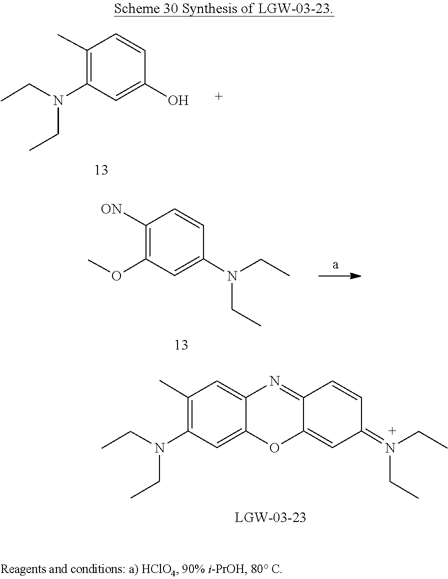

### N-(7-(diethylamino)-8-methyl-3H-phenoxazin-3-ylidene)-N-ethylethanaminium (LGW-03-23)

Compound 13 (50 mg, 0.28 mmol) was dissolved in a solution of i-PrOH/H₂O (9/1, 1 mL) at 80° C. for 30 min. A suspended solution of 35 (61 mg, 0.29 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-23 (60 mg, 56%) as a dark blue solid.

LGW-03-31

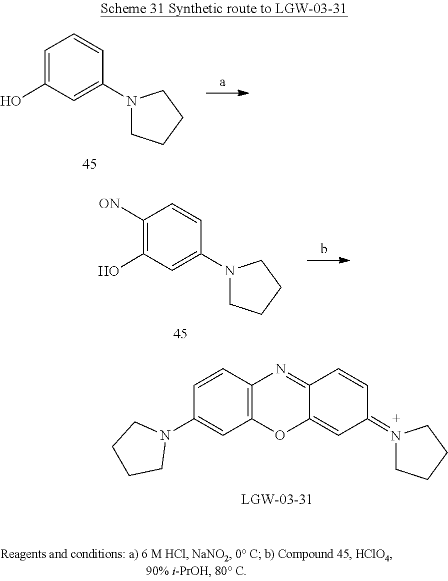

### 2-nitroso-5-(pyrrolidin-1-yl)phenol (48)

Compound 45 (0.4 g, 2.45 mmol) was dissolved in an ice-cold 6 M HCl solution (4 mL). To the solution, NaNO₂ (0.178 g, 2.57 mmol) was added portion wise over 1 h while maintaining the temperature of the solution below 5° C., such that no brown NOx vapors were observed. The reaction mixture was stirred for additional 2 h. After which, the precipitate was filtered through a Büchner funnel and washed with small portions of ice-cold 2 M HCl solution. The product was left in the funnel and air dried overnight to afford compound 48 (0.403 g, 86%) as a bright yellow solid, which was used for the next step without further purification.

### 1-(7-(pyrrolidin-1-yl)-3H-phenoxazin-3-ylidene)pyrrolidin-1-ium (LGW-03-31)

Compound 45 (40 mg, 0.25 mmol) was dissolved in a solution of i-PrOH/H₂O (9/1, 1 mL) at 80° C. for 30 min. A suspended solution of 48 (50 mg, 0.26 mmol) and HClO₄ (70%, 25 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-31 (37 mg, 41%) as a dark blue solid.

LGW-03-32

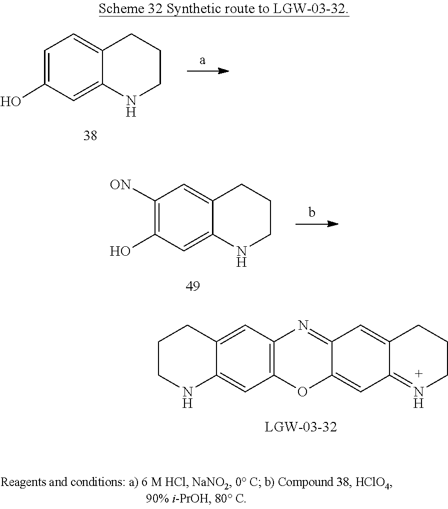

### 6-nitroso-1,2,3,4-tetrahydroquinolin-7-ol (49)

Compound 38 (0.4 g, 2.68 mmol) was dissolved in an ice-cold 6 M HCl solution (4 mL). To the solution, NaNO₂ (0.2 g, 2.95 mmol) was added portion wise over 1 h while maintaining the temperature of the solution below 5° C., such that no brown NOx vapors were observed. The reaction mixture was stirred for additional 2 h. After which, the precipitate was filtered through a Büchner funnel and washed with small portions of ice-cold 2 M HCl solution. The product was left in the funnel and air dried overnight to afford compound 49 (0.418 g, 89%) as a light green solid, which was used for the next step without further purification.

### 3,4,8,9,10,11-hexahydro-2H-dipyrido[3,2-b:2′,3′-i]phenoxazin-1-ium (LGW-03-32)

Compound 38 (40 mg, 0.27 mmol) was dissolved in a solution of i-PrOH/H₂O (9/1, 1 mL) at 80° C. for 30 min. A suspended solution of 49 (50 mg, 0.28 mmol) and HClO₄ (70%, 25 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-32 (31 mg, 40%) as a dark blue solid.

LGW-03-37

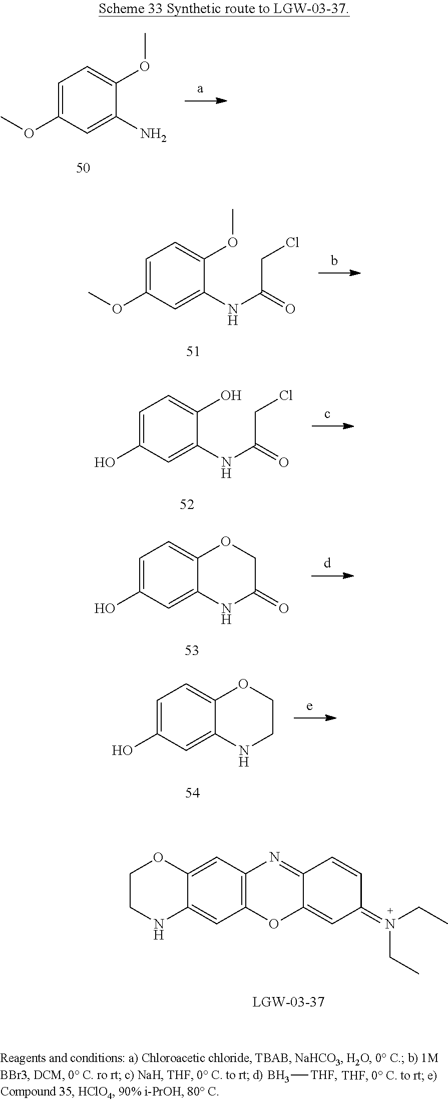

### 2-chloro-N-(2,5-dimethoxyphenyl)acetamide (51)

Compound 50 (20 g, 130 mmol) was dissolved in anhydrous MeCN (60 mL) under N₂, and chilled in an ice bath. To the solution, Et₃N (40 mL, 287 mmol) and Chloroacetic chloride (12.68 mL, 159 mmol) were carefully added. The reaction mixture was stirred for 1 h, then diluted with 500 mL DI water. The solid suspension was filtered off to yield compound 51 (21 g, 70%) as a light brown solid, which was used for the next step without further purification.

### 2-chloro-N-(2,5-dihydroxyphenyl)acetamide (52)

Compound 51 (10 g, 43.54 mmol) was dissolved in anhydrous DCM (20 mL) under N₂, and chilled in an ice bath. To the solution above, was added BBr₃ (1 M in DCM, 130 mL, 130 mmol) dropwise over 1 h using a syringe pump. The reaction mixture was slowly warmed up to rt and stirred overnight. The reaction flask was placed in an ice bath, after sufficient amount of time for cooling, water was carefully added to the reaction mixture to destroy excess BBr₃. The resulting precipitate was collected via vacuum filtration and washed with small portions of DI water. The product was left in the funnel and air dried overnight to afford compound 52 (7.96 g, 91%), which was used for the next step without further purification.

### 6-hydroxy-2H-benzo[b][1,4]oxazin-3(4H)-one (53)

Compound 52 (6 g, 29.76 mmol) was dissolved in anhydrous THF (50 mL) under N₂, and chilled in an ice bath. After sufficient time for cooling, NaH (60%, 4.17 g, 104 mmol) was added to the solution in 4 portions over 10 mins. The reaction mixture was slowly warmed to rt and stirred overnight. Upon the completion of the reaction, ice-cold water was carefully added to the reaction flask to destroy excess NaH. The reaction mixture was acidified with 2 M HCl, followed by extraction with EtOAc (5×100 mL). The combined organic layers were rinsed with brine and dried over anhydrous Na₂SO₄. The solvent was removed using a rotary evaporator and the residue was purified by flash column chromatography with silica gel (100 g), using EtOAc/DCM/Hexane as eluent to give compound 53 (3.12 g, 63%) as a light brown solid.

### 3,4-dihydro-2H-benzo[b][1,4]oxazin-6-ol (54)

A solution of 53 (2.2 g, 13.32 mmol) in anhydrous THF (40 mL) was stirred in an ice bath under N₂ for 30 mins. Borane tetrahydrofuran complex solution (1 M, 40 mL) was added to the solution above using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, the residue was purified by flash column chromatography with silica gel (50 g), using ErOAc/Hexane as eluent to obtain 54 (1.914 g, 95%).

### N-(3,4-dihydro-[1,4]oxazino[2,3-b]phenoxazin-8(2H)-ylidene)-N-ethylethanaminium (LGW-03-37)

Compound 54 (36 mg, 0.24 mmol) was dissolved in a solution of i-PrOH/H₂O (9/1, 1 mL) at 80° C. for 30 min. A suspended solution of 35 (52 mg, 0.25 mmol) and HClO₄ (70%, 25 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-37 (27 mg, 32%) as a dark blue solid.

LGW-03-41

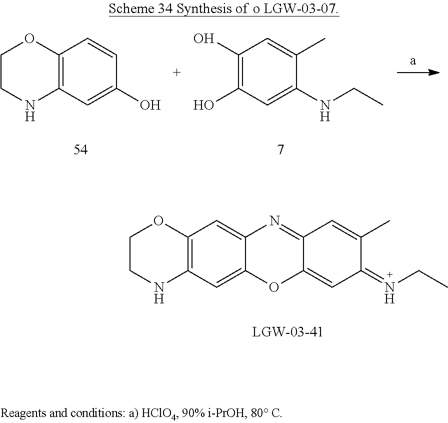 [SMILES: Oc1ccc2c(c1)NCCO2 ; CCNc1cc(O)c(O)cc1C ; CC/N=c1/cc2oc3cc4c(cc3nc-2cc1C)OCCN4]

### (Z)—N-(9-methyl-3,4-dihydro-[1,4]oxazino[2,3-b]phenoxazin-8(2H)-ylidene)ethanaminium (LGW-03-41)

Compound 54 (50 mg, 0.33 mmol) was dissolved in a solution of i-PrOH/H₂O (9/1, 1 mL) at 80° C. for 30 min. A suspended solution of 7 (63 mg, 0.35 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-41 (50 mg, 44%) as a dark blue solid.

LGW-03-52

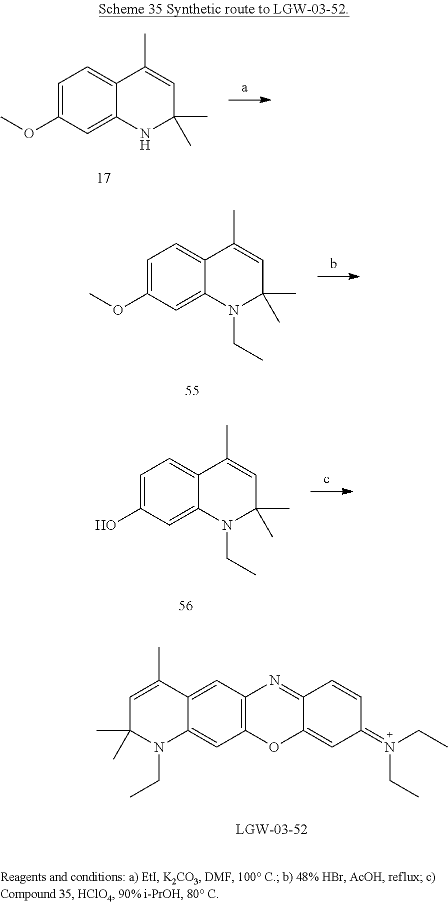

### 1-ethyl-7-methoxy-2,2,4-trimethyl-1,2-dihydroquinoline (55)

To a suspension of compound 17 (4 g, 19.68 mmol) and K₂CO₃ (2.72 g, 19.68 mmol) in anhydrous DMF (20 mL) under N₂, was added Etl (4.75 mL, 59.03 mmol) at rt. The reaction mixture was then heated up to 90° C. and stirred overnight. The solution was cooled down to rt and concentrated under reduced pressure. The crude product was diluted with 100 mL DI water, and the aqueous phase was extracted with EtOAc (4×100 mL). The combined organic layers were rinsed with brine and dried over anhydrous Na₂SO₄. The solvent was removed using a rotary evaporator and the residue was purified by flash column chromatography with silica gel (200 g), using DCM/Hexane as eluent to give compound 55 (3.95 g, 87%) as clear oil.

### 1-ethyl-2,2,4-trimethyl-1,2-dihydroquinolin-7-ol (56)

Compound 55 (0.5 g, 2.16 mmol) was dissolved in glacial AcOH (2 mL) at rt, aqueous HBr (48%, 2 mL) was added to the solution above. The resulting solution was heated at 110° C. for 5 h before cooling. After which the reaction mixture was diluted with 50 mL DI water, and the pH of the solution was adjusted to 5-6 with 2 M NaOH solution. The aqueous solution was extracted with DCM (3×25 mL). The combined organic layers were rinsed with brine and dried over anhydrous Na₂SO₄. The solvent was removed using a rotary evaporator and the residue was purified by flash column chromatography with silica gel (25 g), using EtOAc/Hexane as eluent to give compound 56 (292 mg, 62%).

### N-ethyl-N-(1-ethyl-2,2,4-trimethyl-1,2-dihydro-9H-pyrido[3,2-b]phenoxazin-9-ylidene)ethanaminium (LGW-03-52)

Compound 56 (40 mg, 0.18 mmol) was dissolved in a solution of i-PrOH/H₂O (9/1, 1 mL) at 80° C. for 30 min. A suspended solution of 35 (40 mg, 0.19 mmol) and HClO₄ (70%, 20 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-52 (18 mg, 24%) as a dark blue solid.

LGW-03-57

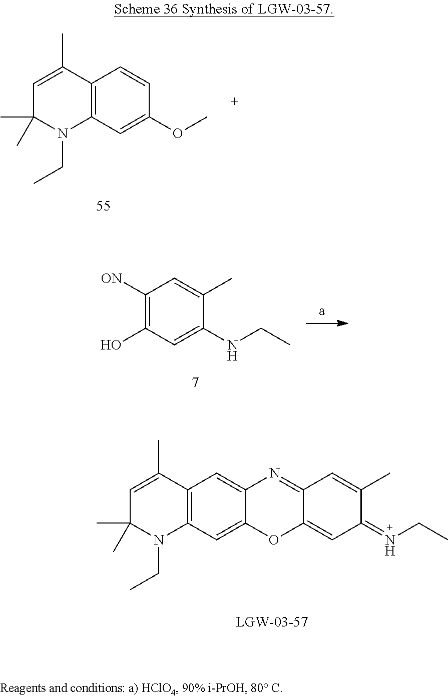

### (Z)—N-(1-ethyl-2,2,4,8-tetramethyl-1,2-dihydro-9H-pyrido[3,2-b]phenoxazin-9-ylidene)ethanaminium (LGW-03-57)

Compound 55 (50 mg, 0.22 mmol) was dissolved in a solution of i-PrOH/H₂O (9/1, 1 mL) at 80° C. for 30 min. A suspended solution of 7 (41 mg, 0.23 mmol) and HClO₄ (70%, 25 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-57 (67 mg, 76%) as a dark blue solid.

LGW-03-65

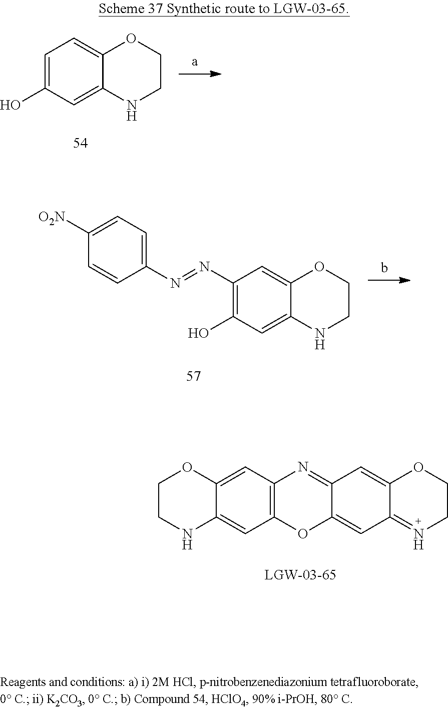 [SMILES: Oc1ccc2c(c1)NCCO2 ; O=[N+]([O-])c1ccc(/N=N/c2cc3c(cc2O)NCCO3)cc1 ; c1c2c(cc3c1Oc1cc4c(cc1=N3)OCCN=4)OCCN2]

### (E)-7-((4-nitrophenyl)diazenyl)-3,4-dihydro-2H-benzo[b][1,4]oxazin-6-ol (57)

Compound 54 (0.2 g, 1.32 mmol) was dissolved in 0.6 mL MeOH. The solution was chilled in an ice bath, then was treated with HCl (2 M, 6 mL). After 15 mins, p-nitrobenzenediazonium tetrafluoroborate (345 mg, 1.46 mmol) was added to the solution in 5 portions over 15 mins, then stirred at 0° C. for additional 1 h. During which time, the color of the reaction mixture changed from orange to dark red. After which, the solution was carefully basified with solid K₂CO₃ until pH value of the solution rose above 8. The deep red precipitate was collected via vacuum filtration and washed with small portions of DI water. The product was left in the funnel and air dried overnight to afford compound 57 (344 mg, 87%), which was used for the next step without further purification.

### 3,8,9,10-tetrahydro-2H-bis([1,4]oxazino)[2,3-b:3′,2′-i]phenoxazin-4-ium (LGW-03-65)

Compound 54 (50 mg, 0.33 mmol) was dissolved in a solution of i-PrOH/H₂O (9/1, 3 mL) at 80° C. for 30 min. Compound 57 (99 mg, 0.33 mmol) was added to the solution above in 5 portions over 15 mins. Then the reaction mixture was treated with HClO₄ (70%, 30 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-65 (62 mg, 55%) as a dark blue solid.

LGW-03-76

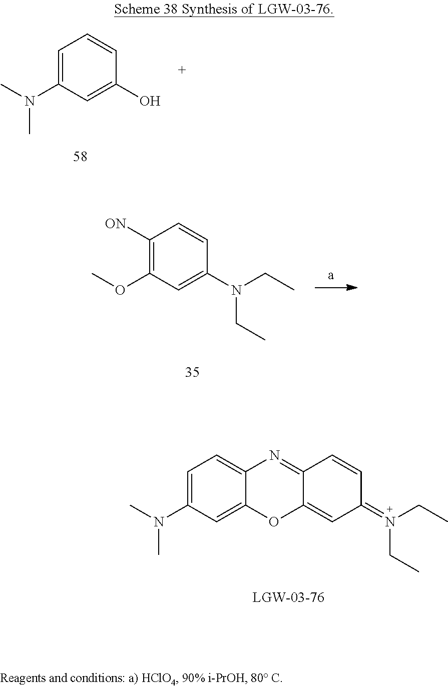

### N-(7-(dimethylamino)-3H-phenoxazin-3-ylidene)-N-ethylethanaminium (LGW-03-76)

Compound 58 (50 mg, 0.36 mmol) was dissolved in a solution of i-PrOH/H₂O (9/1, 1 mL) at 80° C. for 30 min. A suspended solution of 35 (80 mg, 0.38 mmol) and HClO₄ (70%, 35 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-76 (19 mg, 15%) as a dark blue solid.

LGW-03-88

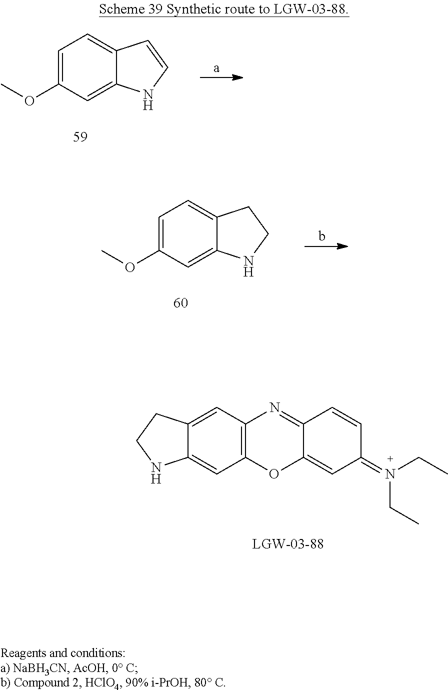

### 6-methoxyindoline (60)

Compound 39 (4 g, 27.18 mmol) was dissolved in acetic acid (10 mL). NaBH₃CN (6.83 g, 108.71 mmol) was added into the reaction flask portion-wise while maintaining the temperature below 10° C. The resulting solution was stirred for 1 h. After which, the solution was diluted with ice-cold water and basified with 2 M NaOH until the pH of the solution rose above 8. The reaction mixture was extracted with EtOAc (4×100 mL). The combined organic layers were rinsed with brine and dried over anhydrous Na₂SO₄. The solvent was removed using a rotary evaporator and the residue was purified by flash column chromatography with silica gel (100 g), using DCM/Hexane as eluent to give compound 60 (3.52 g, 87%) as light brown oil.

### N-(2,3-dihydropyrrolo[3,2-b]phenoxazin-8(1H)-ylidene)-N-ethylethanaminium (LGW-03-88)

Compound 60 (50 mg, 0.34 mmol) was dissolved in a solution of i-PrOH/H₂O (9/1, 1 mL) at 80° C. for 30 min. A suspended solution of 2 (68 mg, 0.35 mmol) and HClO₄ (70%, 35 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-03-88 (59 mg, 52%) as a dark blue solid.

LGW-04-31

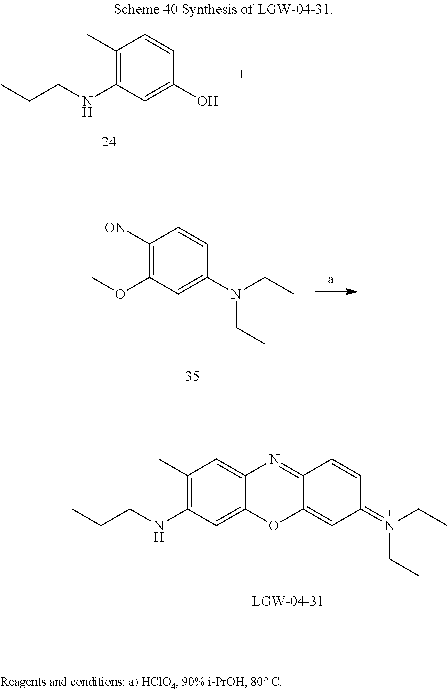

### N-ethyl-N-(8-methyl-7-(propylamino)-3H-phenoxazin-3-ylidene)ethanaminium (LGW-04-31)

Compound 24 (40 mg, 0.24 mmol) was dissolved in a solution of i-PrOH/H₂O (9/1, 1 mL) at 80° C. for 30 min. A suspended solution of 35 (53 mg, 0.25 mmol) and HClO₄ (70%, 25 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-04-31 (53 mg, 59%) as a dark blue solid.

LGW-04-32

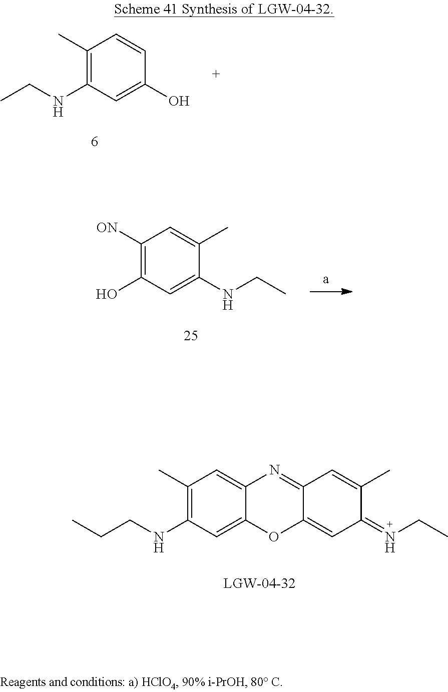

### (Z)—N-(2,8-dimethyl-7-(propylamino)-3H-phenoxazin-3-ylidene)ethanaminium (LGW-04-32)

Compound 6 (50 mg, 0.33 mmol) was dissolved in a solution of i-PrOH/H₂O (9/1, 1 mL) at 80° C. for 30 min. A suspended solution of 25 (67 mg, 0.35 mmol) and HClO₄ (70%, 35 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-04-32 (76 mg, 65%) as a dark blue solid.

LGW-04-36

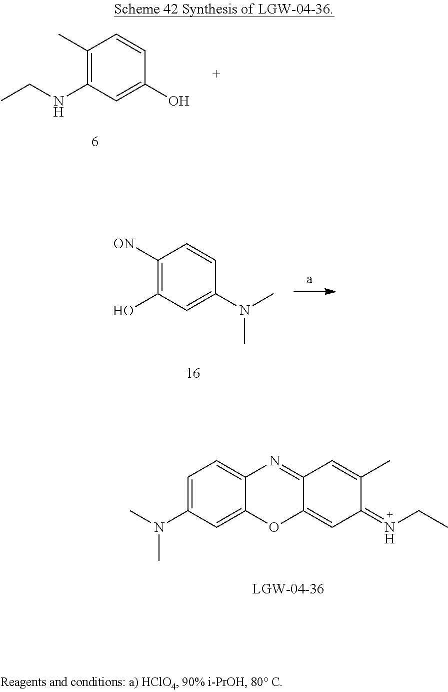

### (Z)—N-(7-(dimethylamino)-2-methyl-3H-phenoxazin-3-ylidene)ethanaminium (LGW-04-36)

Compound 6 (50 mg, 0.33 mmol) was dissolved in a solution of i-PrOH/H₂O (9/1, 1 mL) at 80° C. for 30 min. A suspended solution of 16 (58 mg, 0.35 mmol) and HClO₄ (70%, 35 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-04-36 (52 mg, 48%) as a dark blue solid.

LGW-04-81

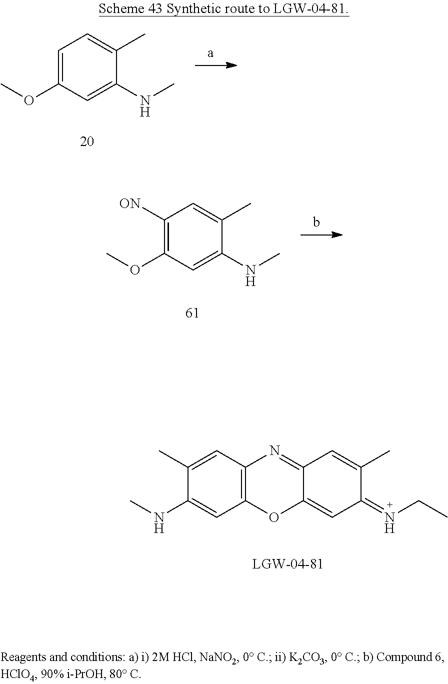

### 5-methoxy-N,2-dimethyl-4-nitrosoaniline (61)

Compound 20 (0.4 g, 2.65 mmol) was dissolved in an ice-cold 2 M HCl solution (5 mL). To the solution above, NaNO₂ (0.2 g, 2.91 mmol) was added portion wise over 1 h while maintaining the temperature of the solution below 5° C., such that no brown NOx vapors were observed. The reaction mixture was stirred for additional 2 h. The solution was carefully basified with solid K₂CO₃ until the pH value of the solution rose above 8. After which, the precipitate was filtered through a Büchner funnel and washed with small portions of DI water. The product was left in the funnel and air dried overnight to afford compound 61 (407 mg, 85%) as a green solid, which was used for the next step without further purification.

### (Z)—N-(2,8-dimethyl-7-(methylamino)-3H-phenoxazin-3-ylidene)ethanaminium (LGW-04-81)

Compound 6 (50 mg, 0.33 mmol) was dissolved in a solution of i-PrOH/H₂O (9/1, 1 mL) at 80° C. for 30 min. A suspended solution of 61 (63 mg, 0.35 mmol) and HClO₄ (70%, 35 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-04-81 (21 mg, 23%) as a dark blue solid.

LGW-04-84

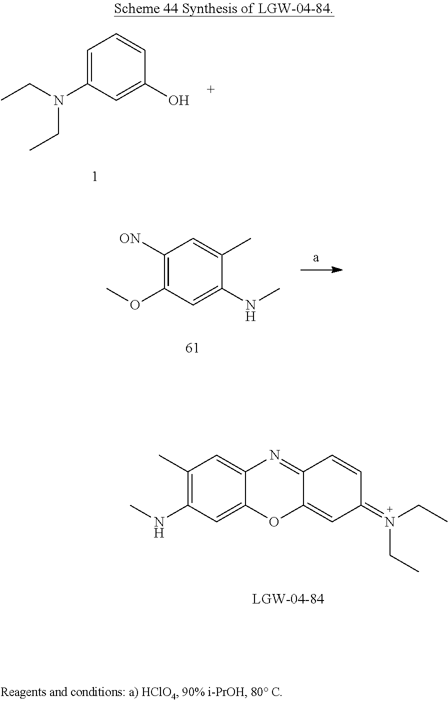

### N-ethyl-N-(8-methyl-7-(methylamino)-3H-phenoxazin-3-ylidene)ethanaminium (LGW-04-84)

Compound 1 (50 mg, 0.3 mmol) was dissolved in a solution of i-PrOH/H₂O (9/1, 1 mL) at 80° C. for 30 min. A suspended solution of 61 (57 mg, 0.32 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-04-84 (36 mg, 35%) as a dark blue solid.

LGW-04-91

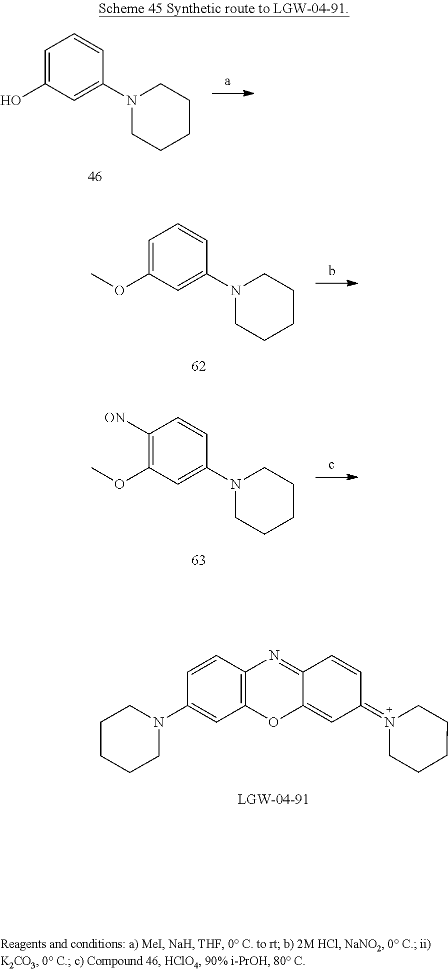

### 1-(3-methoxyphenyl)piperidine (62)

Compound 46 (0.5 g, 2.82 mmol) was dissolved in anhydrous THF (5 mL) under N₂, and chilled in an ice bath for 30 mins. NaH (60%, 0.136 g, 3.39 mmol) was added to the solution in 3 portions over 10 mins while the temperature maintained below 5° C. After 10 mins, Mel (0.211 mL, 3.39 mmol) was added into the reaction mixture. The resulting suspension was slowly warmed up to rt and stirred overnight. Upon completion of the reaction, ice-cold DI water was added to the reaction mixture to destroy excess NaH. Organic solvent was removed under reduced pressure, and the residue was extracted with DCM (3×25 mL). The combined organic layers were rinsed with brine and dried over anhydrous Na₂SO₄. The solvent was removed using a rotary evaporator and the residue was purified by flash column chromatography with silica gel (25 g), using DCM/Hexane as eluent to give compound 62 (0.447 g, 83%).

### 1-(3-methoxy-4-nitrosophenyl)piperidine (63)

Compound 62 (0.1 g, 0.52 mmol) was dissolved in an ice-cold 2 M HCl solution (2 mL). To the solution above, NaNO₂ (0.04 g, 0.58 mmol) was added portion wise over 1 h while maintaining the temperature of the solution below 5° C., such that no brown NOx vapors were observed. The reaction mixture was stirred for additional 2 h. The solution was carefully basified with solid K₂CO₃ until pH value of the solution rose above 8. After which, the aqueous solution was extracted with DCM (3×25 mL). The combined organic layers were rinsed with brine and dried over anhydrous Na₂SO₄. The solvent was removed using a rotary evaporator to give 63 (102 mg, 89%) as a light green oil, which was used for the next step without further purification.

### 1-(7-(piperidin-1-yl)-3H-phenoxazin-3-ylidene)piperidin-1-ium (LGW-04-91)

Compound 46 (50 mg, 0.28 mmol) was dissolved in a solution of i-PrOH/H₂O (9/1, 1 mL) at 80° C. for 30 min. A suspended solution of 63 (65 mg, 0.30 mmol) and HClO₄ (70%, 30 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-04-91 (28 mg, 26%) as a dark blue solid.

LGW-05-33

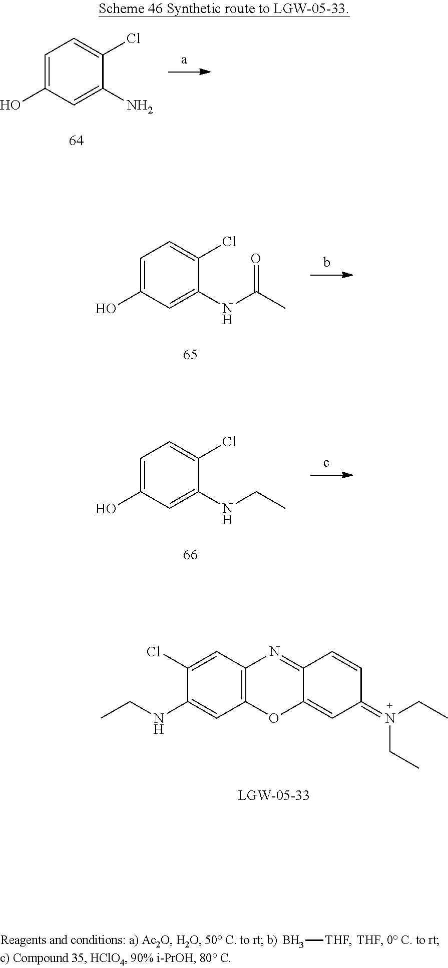

### N-(2-chloro-5-hydroxyphenyl)acetamide (65)

Compound 64 (1 g, 6.97 mmol) was suspended in 10 mL DI water, to which Acetic anhydride (2.6 mL, 27.86 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of DI water. The product was left in the funnel and air dried overnight to afford compound 65 (1.03 g, 80%) as a white solid, which was used for the next step without further purification.

### 4-chloro-3-(ethylamino)phenol (66)

A solution of 65 (1 g, 5.39 mmol) in anhydrous THF (16 mL) was stirred in an ice bath under N₂ for 30 mins. Borane tetrahydrofuran complex solution (1 M, 16 mL) was added to the solution above using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, the residue was purified by flash column chromatography with silica gel (25 g), using DCM/Hexane as eluent to obtain 66 (0.82 g, 89%) as a white solid.

### N-(8-chloro-7-(ethylamino)-3H-phenoxazin-3-ylidene)-N-ethylethanaminium (LGW-05-33)

Compound 66 (40 mg, 0.23 mmol) was dissolved in a solution of i-PrOH/H₂O (9/1, 1 mL) at 80° C. for 30 min. A suspended solution of 35 (51 mg, 0.24 mmol) and HClO₄ (70%, 25 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-33 (13 mg, 15%) as a dark blue solid.

LGW-05-39

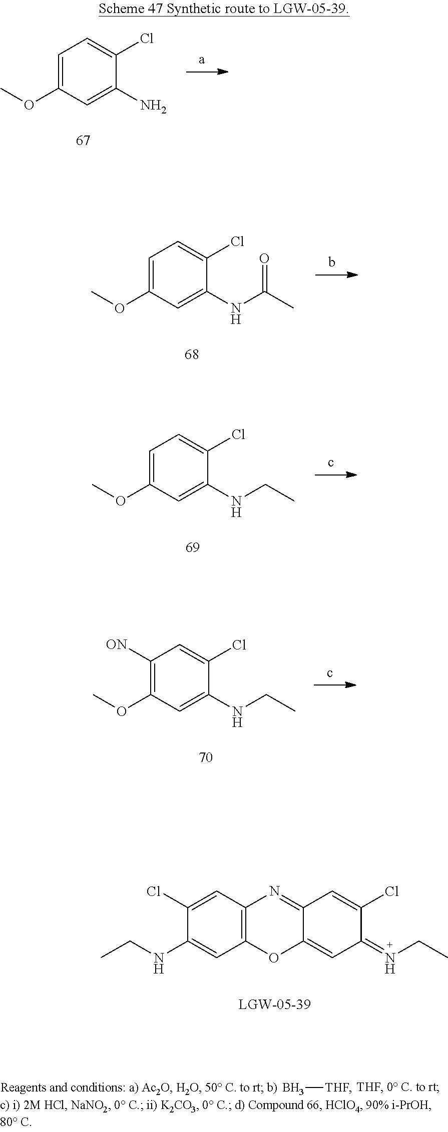

### N-(2-chloro-5-methoxyphenyl)acetamide (68)

Compound 67 (4 g, 25.38 mmol) was suspended in 40 mL DI water, to which Acetic anhydride (9.6 mL, 102 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of DI water. The product was left in the funnel and air dried overnight to afford compound 68 (4.91 g, 97%) as a white solid, which was used for the next step without further purification.

### 2-chloro-N-ethyl-5-methoxyaniline (69)

A solution of 68 (2 g, 10 mmol) in anhydrous THF (30 mL) was stirred in an ice bath under N₂ for 30 mins. Borane tetrahydrofuran complex solution (1 M, 30 mL) was added to the solution using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, the residue was purified by flash column chromatography with silica gel (25 g), using DCM/Hexane as eluent to obtain 69 (1.43 g, 77%) as a white solid.

### 2-chloro-N-ethyl-5-methoxy-4-nitrosoaniline (70)

Compound 69 (0.5 g, 2.69 mmol) was dissolved in an ice-cold 2 M HCl solution (10 mL). NaNO₂ (0.2 g, 2.96 mmol) was added portion wise over 1 h while maintaining the temperature of the solution below 5° C., such that no brown NOx vapors were observed. The reaction mixture was stirred for additional 2 h. The solution was carefully basified with solid K₂CO₃ until pH value of the solution rose above 8. After which, the aqueous solution was extracted with DCM (3×25 mL). The combined organic layers were rinsed with brine and dried over anhydrous Na₂SO₄. The solvent was removed using a rotary evaporator to give 63 (469 mg, 81%) as dark green oil, which was used for the next step without further purification.

### (Z)—N-(2,8-dichloro-7-(ethylamino)-3H-phenoxazin-3-ylidene)ethanaminium (LGW-05-39)

Compound 66 (60 mg, 0.35 mmol) was dissolved in a solution of i-PrOH/H₂O (9/1, 1 mL) at 80° C. for 30 min. A suspended solution of 70 (79 mg, 0.37 mmol) and HClO₄ (70%, 35 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-39 (99 mg, 74%) as a dark blue solid.

LGW-05-42

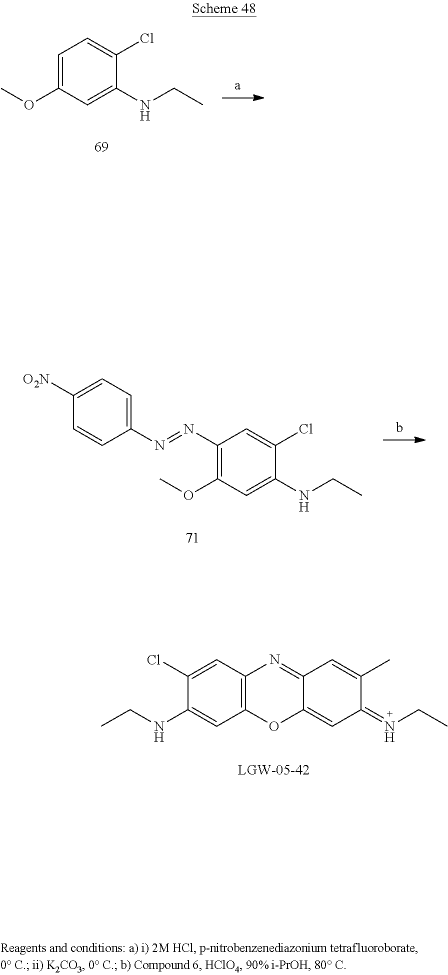

### (E)-2-chloro-N-ethyl-5-methoxy-4-((4-nitrophenyl)diazenyl)aniline (71)

Compound 69 (0.5 g, 2.69 mmol) was dissolved in 1.5 mL MeOH. The solution was chilled in an ice bath, then was treated with HCl (2 M, 13 mL). After 15 mins, p-nitrobenzenediazonium tetrafluoroborate (702 mg, 2.96 mmol) was added in 5 portions over 15 mins, then stirred at 0° C. for additional 1 h. During which time, the color of the reaction mixture changed from orange to dark red. After which, the solution was carefully basified with solid K₂CO₃ until pH value of the solution rose above 8. The deep red precipitate was collected via vacuum filtration and washed with small portions of DI water. The product was left in the funnel and air dried overnight to afford compound 71 (812 mg, 90%) as a red solid, which was used for the next step without further purification.

### (Z)—N-(8-chloro-7-(ethylamino)-2-methyl-3H-phenoxazin-3-ylidene)ethanaminium (LGW-05-42)

Compound 6 (45 mg, 0.3 mmol) was dissolved in a solution of i-PrOH/H₂O (9/1, 3 mL) at 80° C. for 30 min. Compound 71 (100 mg, 0.3 mmol) was added to the solution in 5 portions over 15 mins. Then the reaction mixture was treated with HClO₄ (70%, 30 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-42 (22 mg, 21%) as a dark blue solid.

LGW-05-65

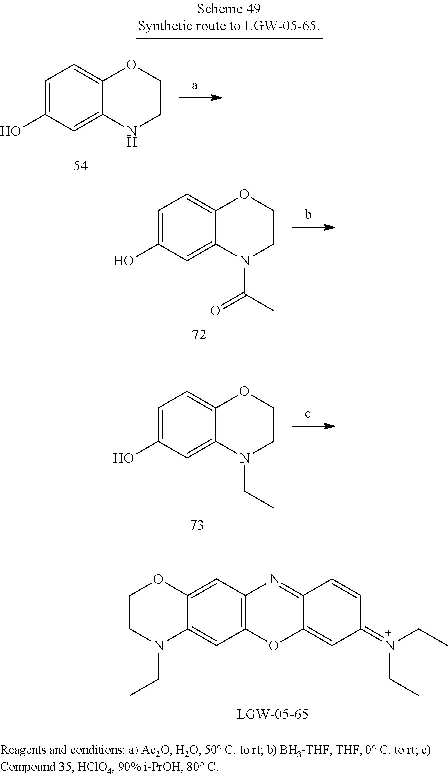 [SMILES: Oc1ccc2c(c1)NCCO2 ; CC(=O)N1CCOc2ccc(O)cc21 ; CCN1CCOc2ccc(O)cc21 ; CCN1CCOc2cc3nc4ccc(=[N+](CC)CC)cc-4oc3cc21]

### 1-(6-hydroxy-2,3-dihydro-4H-benzo[b][1,4]oxazin-4-yl)ethan-1-one (72)

Compound 54 (1 g, 6.62 mmol) was suspended in 10 mL DI water, to which Acetic anhydride (2.5 mL, 26.46 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of DI water. The product was left in the funnel and air dried overnight to afford compound 72 (1.21 g, 95%) as a white solid, which was used for the next step without further purification.

### 4-ethyl-3,4-dihydro-2H-benzo[b][1,4]oxazin-6-ol (73)

A solution of 72 (0.9 g, 10 mmol) in anhydrous THF (14 mL) was stirred in an ice bath under N₂ for 30 mins. Borane tetrahydrofuran complex solution (1 M, 14 mL) was added to the solution using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, the residue was purified by flash column chromatography with silica gel (25 g), using DCM/Hexane as eluent to give 73 (756 mg, 91%) as brown oil.

### N-ethyl-N-(4-ethyl-3,4-dihydro-[1,4]oxazino[2,3-b]phenoxazin-8(2H)-ylidene)ethanaminium (LGW-05-65)

Compound 73 (50 mg, 0.28 mmol) was dissolved in a solution of i-PrOH/H₂O (9/1, 1 mL) at 80° C. for 30 min. A suspended solution of 35 (61 mg, 0.29 mmol) and HClO₄ (70%, 30 μL) in 90% i-PrOH (2 mL) was in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-65 (81 mg, 76%) as a dark blue solid.

LGW-05-66

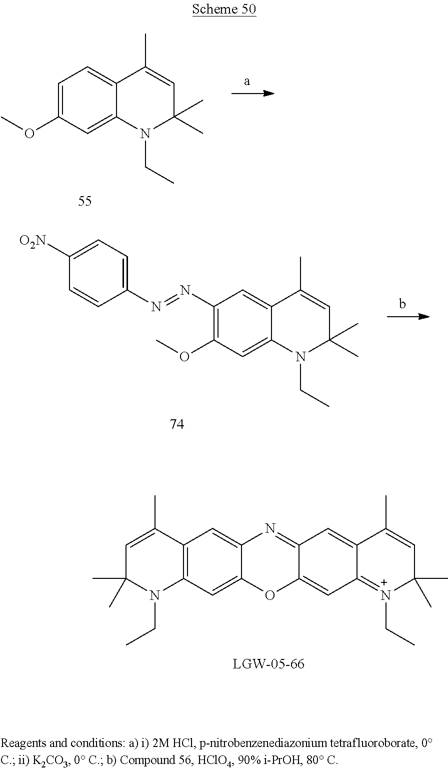

### (E)-1-ethyl-7-methoxy-2,2,4-trimethyl-6-((4-nitrophenyl)diazenyl)-1,2-dihydroquinoline (74)

Compound 55 (0.2 g, 0.86 mmol) was dissolved in 1 mL MeOH. The solution was chilled in an ice bath, then treated with HCl (2 M, 5 mL). After 15 mins, p-nitrobenzenediazonium tetrafluoroborate (215 mg, 0.91 mmol) was added to the solution in 5 portions over 15 mins, then stirred at 0° C. for additional 1 h. During which time, the color of the reaction mixture changed from orange to dark red. After which, the solution was carefully basified with solid K₂CO₃ until the pH value of the solution rose above 8. The deep red precipitate was collected via vacuum filtration and washed with small portions of DI water. The product was left in the funnel and air dried overnight to afford compound 74 (305 mg, 93%) as a deep red solid, which was used for the next step without further purification.

### 1,11-diethyl-2,2,4,8,10,10-hexamethyl-10,11-dihydro-2H-dipyrido[3,2-b:2′,3′-i]phenoxazin-1-ium (LGW-05-66)

Compound 56 (40 mg, 0.18 mmol) was dissolved in a solution of i-PrOH/H₂O (9/1, 3 mL) at 80° C. for 30 min. Compound 74 (70 mg, 0.18 mmol) was added to the solution in 5 portions over 15 mins. Then the reaction mixture was treated with HClO₄ (70%, 20 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-66 (11 mg, 13%) as a dark blue solid.

LGW-05-73

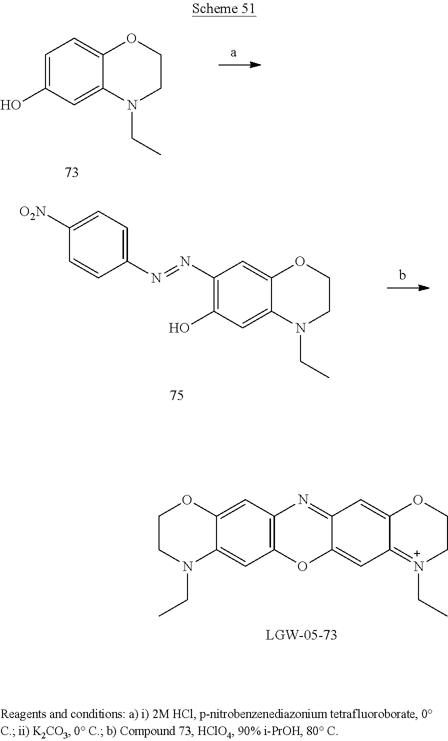 [SMILES: CCN1CCOc2ccc(O)cc21 ; CCN1CCOc2cc(/N=N/c3ccc([N+](=O)[O-])cc3)c(O)cc21 ; CCN1CCOc2cc3c(cc21)Oc1cc2c(cc1=N3)OCC[N+]=2CC]

### (E)-4-ethyl-7-((4-nitrophenyl)diazenyl)-3,4-dihydro-2H-benzo[b][1,4]oxazin-6-ol (75)

Compound 73 (0.2 g, 1.12 mmol) was dissolved in 1 mL MeOH. The solution was chilled in an ice bath, then was treated with HCl (2 M, 5 mL). After 15 mins, p-nitrobenzenediazonium tetrafluoroborate (290 mg, 1.23 mmol) was added to the solution in 5 portions over 15 mins, then stirred at 0° C. for an additional 1 h. During which time, the color of the reaction mixture changed from orange to dark red. After which, the solution was carefully basified with solid K₂CO₃ until pH value of the solution rose above 8. The deep red precipitate was collected via vacuum filtration and washed with small portions of DI water. The product was left in the funnel and air dried overnight to afford compound 75 (319 mg, 87%), which was used for the next step without further purification.

### 4,8-diethyl-3,8,9,10-tetrahydro-2H-bis([1,4]oxazino)[2,3-b:3′,2′-i]phenoxazin-4-ium (LGW-05-73)

Compound 73 (40 mg, 0.22 mmol) was dissolved in a solution of i-PrOH/H₂O (9/1, 3 mL) at 80° C. for 30 min. Compound 75 (73 mg, 0.22 mmol) was added to the solution in 5 portions over 15 mins. Then the reaction mixture was treated with HClO₄ (70%, 20 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-72 (12 mg, 14%) as a dark blue solid.

LGW-05-75

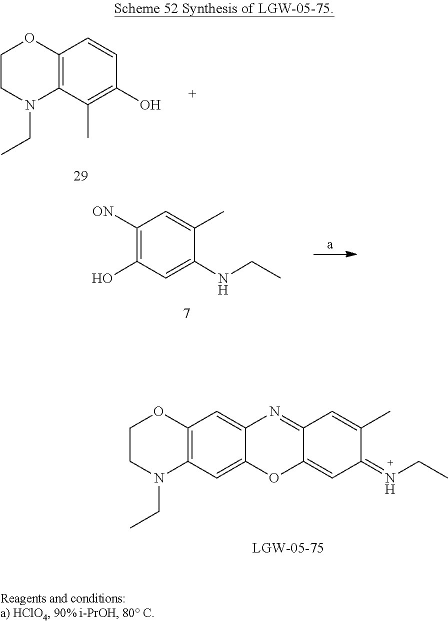

### (Z)—N-(4-ethyl-9-methyl-3,4-dihydro-[1,4]oxazino[2,3-b]phenoxazin-8(2H)-ylidene)ethanaminium (LGW-05-75)

Compound 73 (50 mg, 0.28 mmol) was dissolved in a solution of i-PrOH/H₂O (9/1, 1 mL) at 80° C. for 30 min. A suspended solution of 7 (53 mg, 0.29 mmol) and HClO₄ (70%, 35 μL) in 90% i-PrOH (2 mL) was added into the solution in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-75 (79 mg, 77%) as a dark blue solid.

LGW-05-76

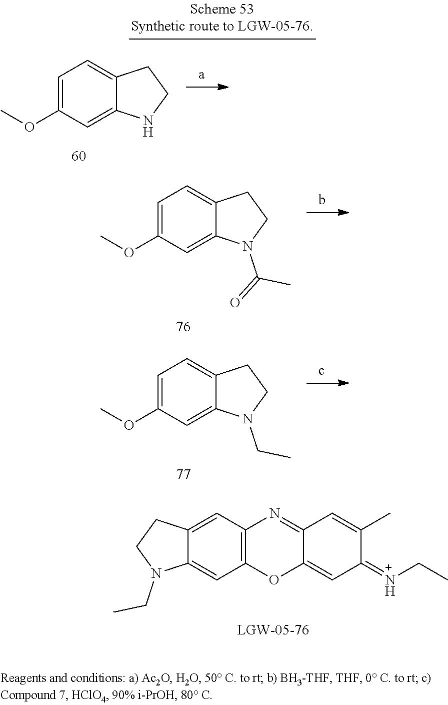 [SMILES: COc1ccc2c(c1)NCC2 ; COc1ccc2c(c1)N(C(C)=O)CC2 ; CCN1CCc2ccc(OC)cc21 ; CC/[NH+]=c1/cc2oc3cc4c(cc3nc-2cc1C)CCN4CC]

### 1-(6-methoxyindolin-1-yl)ethan-1-one (76)

Compound 60 (1 g, 6.7 mmol) was suspended in 10 mL DI water, to which Acetic anhydride (2.53 mL, 26.81 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of DI water. The product was left in the funnel and air dried overnight to afford compound 76 (1.12 g, 87%) as a white solid, which was used for the next step without further purification.

### 1-ethyl-6-methoxyindoline (77)

A solution of 76 (1 g, 5.23 mmol) in anhydrous THF (16 mL) was stirred in an ice bath under N₂ for 30 mins. Borane tetrahydrofuran complex solution (1 M, 16 mL) was added to the solution above using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, the residue was purified by flash column chromatography with silica gel (25 g), using DCM/Hexane as eluent to give 77 (861 mg, 93%).

### (Z)—N-(1-ethyl-7-methyl-2,3-dihydropyrrolo[3,2-b]phenoxazin-8(1H)-ylidene)ethanaminium (LGW-05-76)

Compound 77 (40 mg, 0.23 mmol) was dissolved in a solution of i-PrOH/H₂O (9/1, 1 mL) at 80° C. for 30 min. A suspended solution of 7 (43 mg, 0.24 mmol) and HClO₄ (70%, 25 μL) in 90% i-PrOH (2 mL) was added into the solution in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-76 (20 mg, 25%) as a dark blue solid.

LGW-05-81

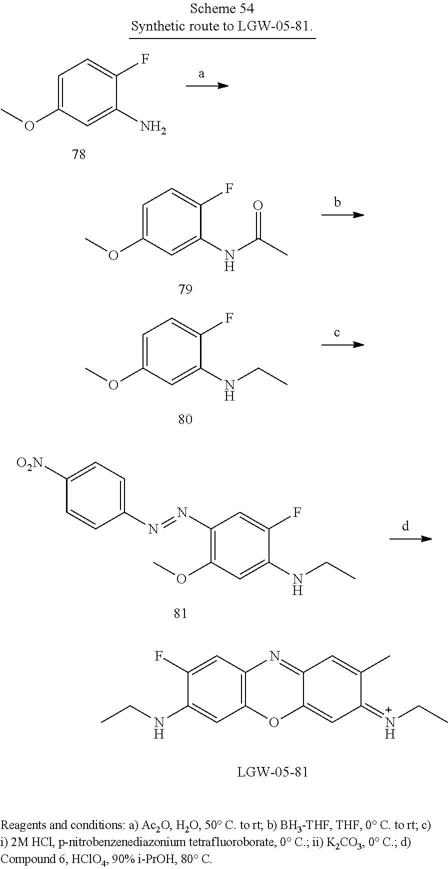

### N-(2-fluoro-5-methoxyphenyl)acetamide (79)

Compound 78 (4 g, 28.34 mmol) was suspended in 40 mL DI water, to which Acetic anhydride (10.72 mL, 113.4 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of DI water. The product was left in the funnel and air dried overnight to afford compound 79 (4.71 g, 91%) as a brown solid, which was used for the next step without further purification.

### N-ethyl-2-fluoro-5-methoxyaniline (80)

A solution of 79 (2 g, 10.92 mmol) in anhydrous THF (33 mL) was stirred in an ice bath under N₂ for 30 mins. Borane tetrahydrofuran complex solution (1 M, 33 mL) was added to the solution above using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, the residue was purified by flash column chromatography with silica gel (25 g), using DCM/Hexane as eluent to give 80 (1.69 g, 92%) as brown oil.

### (E)-N-ethyl-2-fluoro-5-methoxy-4-((4-nitrophenyl)diazenyl)aniline (81)

Compound 80 (0.2 g, 1.18 mmol) was dissolved in 1 mL MeOH. The solution was chilled in an ice bath, then was treated with HCl (2 M, 6 mL). After 15 mins, p-nitrobenzenediazonium tetrafluoroborate (308 mg, 1.3 mmol) was added to the solution in 5 portions over 15 mins, then stirred at 0° C. for additional 1 h. During which time, the color of the reaction mixture changed from orange to dark red. After which, the solution was carefully basified with solid K₂CO₃ until the pH value of the solution rose above 8. The deep red precipitate was collected via vacuum filtration and washed with small portions of DI water. The product was left in the funnel and air dried overnight to afford compound 81 (325 mg, 86%) as a dark red solid, which was used for the next step without further purification.

### (Z)—N-(7-(ethylamino)-8-fluoro-2-methyl-3H-phenoxazin-3-ylidene)ethanaminium (LGW-05-81)

Compound 6 (40 mg, 0.26 mmol) was dissolved in a solution of i-PrOH/H₂O (9/1, 3 mL) at 80° C. for 30 min. Compound 81 (84 mg, 0.26 mmol) was added to the solution in 5 portions over 15 mins. Then the reaction mixture was treated with HClO₄ (70%, 25 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-81 (60 mg, 66%) as a dark blue solid.

LGW-05-82

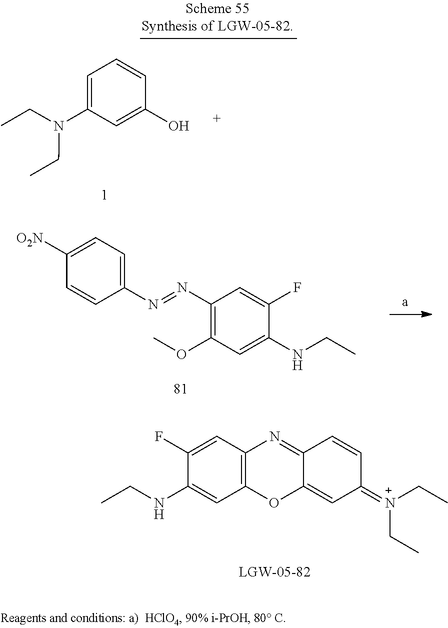

### N-ethyl-N-(7-(ethylamino)-8-fluoro-3H-phenoxazin-3-ylidene)ethanaminium (LGW-05-82)

Compound 1 (40 mg, 0.24 mmol) was dissolved in a solution of i-PrOH/H₂O (9/1, 3 mL) at 80° C. for 30 min. Compound 81 (77 mg, 0.24 mmol) was added to the solution in 5 portions over 15 mins. Then the reaction mixture was treated with HClO₄ (70%, 25 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-82 (17 mg, 20%) as a dark blue solid.

LGW-05-84

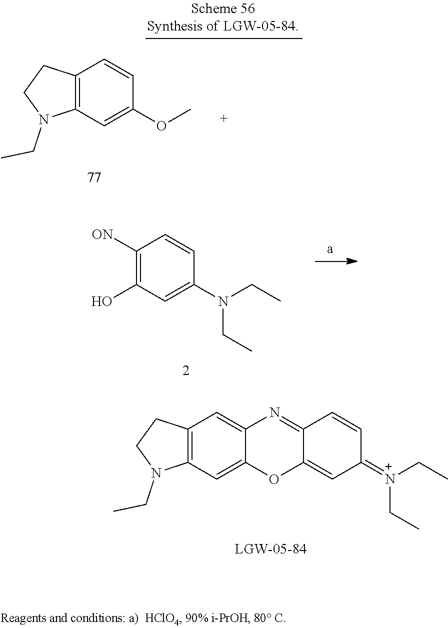

### N-ethyl-N-(1-ethyl-2,3-dihydropyrrolo[3,2-b]phenoxazin-8(1H)-ylidene)ethanaminium (LGW05-84)

Compound 77 (100 mg, 0.56 mmol) was dissolved in a solution of i-PrOH/H₂O (9/1, 2 mL) at 80° C. for 30 min. A suspended solution of 2 (115 mg, 0.24 mmol) and HClO₄ (70%, 55 μL) in 90% i-PrOH (2 mL) was added into the solution above in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-84 (103 mg, 50%) as a dark blue solid.

LGW-05-85

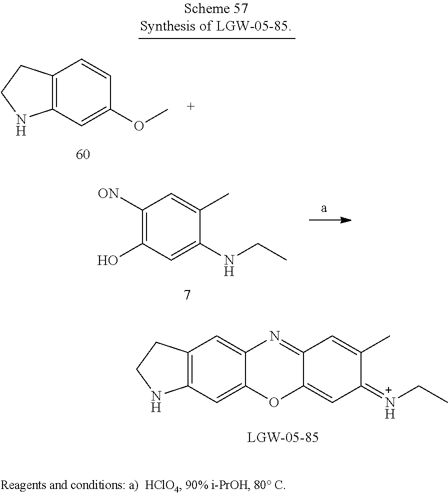

### (Z)—N-(7-methyl-2,3-dihydropyrrolo[3,2-b]phenoxazin-8(1H)-ylidene)ethanaminium (LGW-05-85)

Compound 60 (40 mg, 0.27 mmol) was dissolved in a solution of i-PrOH/H₂O (9/1, 1 mL) at 80° C. for 30 min. A suspended solution of 7 (51 mg, 0.28 mmol) and HClO₄ (70%, 55 μL) in 90% i-PrOH (2 mL) was added into the solution in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-85 (12 mg, 13%) as a dark blue solid.

LGW-05-91

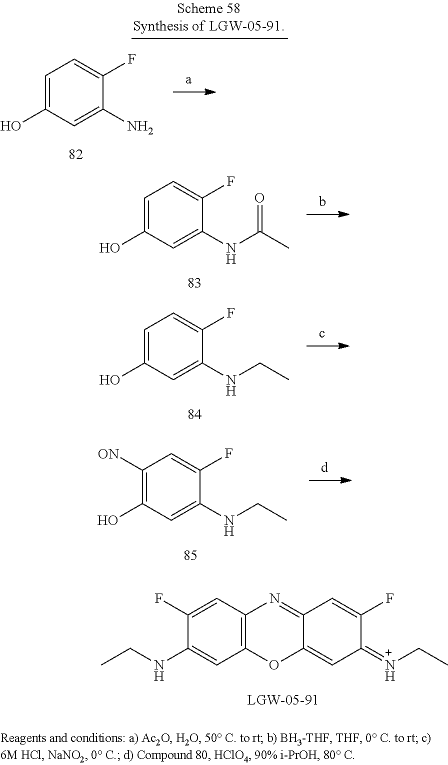

### N-(2-fluoro-5-hydroxyphenyl)acetamide (83)

Compound 82 (4 g, 31.47 mmol) was suspended in 40 mL DI water, to which Acetic anhydride (11.9 mL, 125.87 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of DI water. The product was left in the funnel and air dried overnight to afford compound 83 (4.89 g, 92%) as a light gray solid, which was used for the next step without further purification.

### 3-(ethylamino)-4-fluorophenol (84)

A solution of 83 (2 g, 11.82 mmol) in anhydrous THF (36 mL) was stirred in an ice bath under N₂ for 30 mins. Borane tetrahydrofuran complex solution (1 M, 36 mL) was added to the solution using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, the residue was purified by flash column chromatography with silica gel (25 g), using DCM/Hexane as eluent to obtain 84 (1.61 g, 88%) as a brown solid.

### 5-(ethylamino)-4-fluoro-2-nitrosophenol (85)

Compound 84 (0.5 g, 3.22 mmol) was dissolved in an ice-cold 6 M HCl solution (5 mL). NaNO₂ (233 mg, 3.38 mmol) was added portion wise over 1 h while maintaining the temperature of the solution below 5° C., such that no brown NOx vapors were observed. The reaction mixture was stirred for additional 2 h. After which, the precipitate was filtered through a Büchner funnel and washed with small portions of ice-cold 2 M HCl solution. The product was left in the funnel and air dried overnight to afford compound 85 (419 mg, 71%) as a brown solid, which was used for the next step without further purification.

### (Z)—N-(7-(ethylamino)-2,8-difluoro-3H-phenoxazin-3-ylidene)ethanaminium (LGW-05-91)

Compound 80 (30 mg, 0.18 mmol) was dissolved in a solution of i-PrOH/H₂O (9/1, 1 mL) at 80° C. for 30 min. A suspended solution of 85 (34 mg, 0.19 mmol) and HClO₄ (70%, 55 μL) in 90% i-PrOH (2 mL) was added in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-05-91 (3.5 mg, 6%) as a dark blue solid.

LGW-06-10

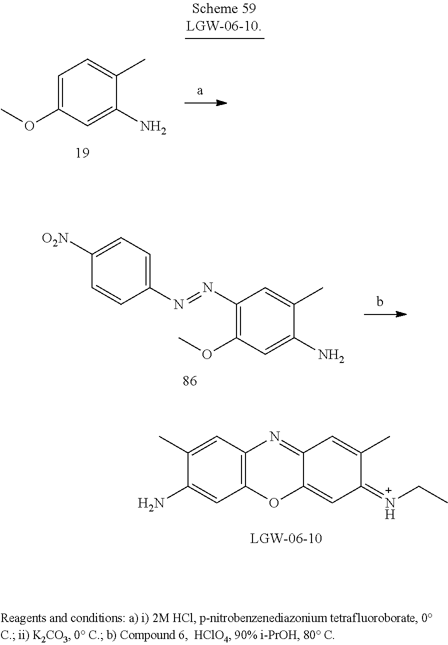

### (E)-5-methoxy-2-methyl-4-((4-nitrophenyl)diazenyl)aniline (86)

Compound 19 (0.2 g, 1.46 mmol) was dissolved in 1 mL MeOH. The solution was chilled in an ice bath, then was treated with HCl (2 M, 7 mL). After 15 mins, p-nitrobenzenediazonium tetrafluoroborate (380 mg, 1.6 mmol) was added to the solution in 5 portions over 15 mins, then stirred at 0° C. for an additional 1 h. During which time, the color of the reaction mixture changed from orange to dark red. After which, the solution was carefully basified with solid K₂CO₃ until pH value of the solution rose above 8. The deep red precipitate was collected via vacuum filtration and washed with small portions of DI water. The product was left in the funnel and air dried overnight to afford compound 86 (316 mg, 76%) as a dark red solid, which was used for the next step without further purification.

### (Z)—N-(7-amino-2,8-dimethyl-3H-phenoxazin-3-ylidene)ethanaminium (LGW-06-10)

Compound 6 (50 mg, 0.33 mmol) was dissolved in a solution of i-PrOH/H₂O (9/1, 2 mL) at 80° C. for 30 min. Compound 86 (95 mg, 0.33 mmol) was added to the solution in 5 portions over 15 mins. Then the reaction mixture was treated with HClO₄ (70%, 30 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-06-10 (50 mg, 49%) as a dark blue solid.

LGW-06-11

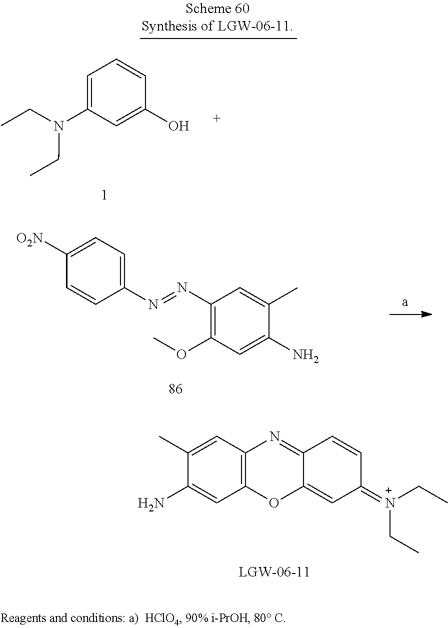 [SMILES: CCN(CC)c1cccc(O)c1 ; COc1cc(N)c(C)cc1/N=N/c1ccc([N+](=O)[O-])cc1 ; CC[N+](CC)=c1ccc2nc3cc(C)c(N)cc3oc-2c1]

### N-(7-amino-8-methyl-3H-phenoxazin-3-ylidene)-N-ethylethanaminium (LGW-06-11)

Compound 1 (40 mg, 0.24 mmol) was dissolved in a solution of i-PrOH/H₂O (9/1, 2 mL) at 80° C. for 30 min. Compound 86 (69 mg, 0.24 mmol) was added to the solution in 5 portions over 15 mins. Then the reaction mixture was treated with HClO₄ (70%, 25 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-06-11 (12 mg, 15%) as a dark blue solid.

LGW-06-14

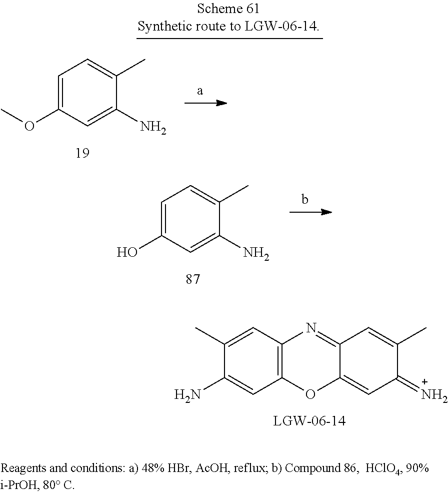

### 3-amino-4-methylphenol (87)

Compound 61 (0.5 g, 3.64 mmol) was dissolved in glacial AcOH (3 mL) at rt and aqueous HBr (48%, 3 mL) was added to the solution. The resulting solution was heated at 110° C. for 5 h before cooling. After which, the reaction mixture was diluted with 20 mL DI water, and the pH of the solution was adjusted to 5-6 with 2 M NaOH solution. The aqueous solution was extracted with DCM (3×25 mL). The combined organic layers were rinsed with brine and dried over anhydrous Na₂SO₄. The solvent was removed using a rotary evaporator and the residue was purified by flash column chromatography with silica gel (25 g), using EtOAc/Hexane as eluent to give compound 87 (288 mg, 64%) as a dark red solid.

### 7-amino-2,8-dimethyl-3H-phenoxazin-3-iminium (LGW-06-14)

Compound 87 (40 mg, 0.32 mmol) was dissolved in a solution of i-PrOH/H₂O (9/1, 2 mL) at 80° C. for 30 min. Compound 86 (93 mg, 0.32 mmol) was added to the solution in 5 portions over 15 mins. Then the reaction mixture was treated with HClO₄ (70%, 30 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-06-14 (13 mg, 14%) as a dark blue solid.

LGW-06-97

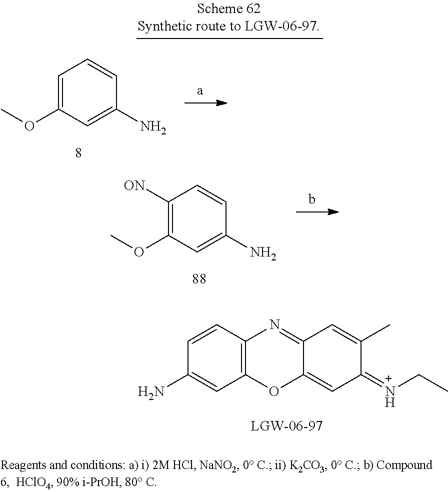

### 3-methoxy-4-nitrosoaniline (88)

Compound 8 (0.909 mL, 7.88 mmol) was dissolved in an ice-cold 2 M HCl solution (25 mL). To the solution above, NaNO₂ (598 mg, 8.66 mmol) was added portion wise over 1 h while maintaining the temperature of the solution below 5° C., such that no brown NOx vapors were observed. The reaction mixture was stirred for an additional 2 h. The solution was carefully basified with solid K₂CO₃ until the pH value of the solution rose above 8. After which, the precipitate was filtered through a Büchner funnel and washed with small portions of DI water. The product was left in the funnel and air dried overnight to afford compound 88 (937 mg, 78%) as a brown solid, which was used for the next step without further purification.

### (Z)—N-(7-amino-2-methyl-3H-phenoxazin-3-ylidene)ethanaminium (LGW-06-97)

Compound 6 (50 mg, 0.33 mmol) was dissolved in a solution of i-PrOH/H₂O (9/1, 2 mL) at 80° C. for 30 min. A suspended solution of 88 (53 mg, 0.35 mmol) and HClO₄ (70%, 35 μL) in 90% i-PrOH (2 mL) was added into the solution in 4 portions over 1 h. The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-06-97 (13 mg, 15%) as a dark blue solid.

LGW-07-55

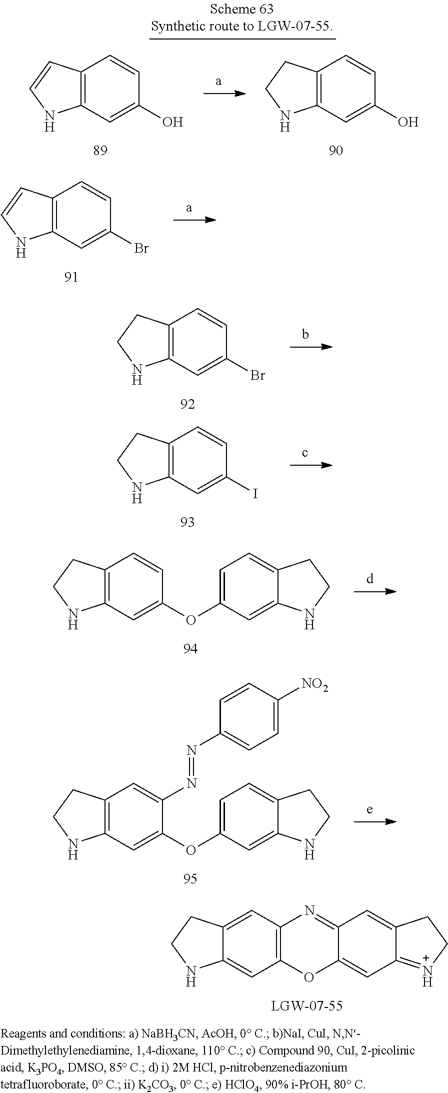

### indolin-6-ol (90)

Compound 89 (2 g, 15.02 mmol) was dissolved in acetic acid (5 mL). NaBH₃CN (2.83 g, 45.06 mmol) was added into the reaction flask portion-wise while maintaining the temperature below 10° C. The resulting solution was stirred for 1 h. After which, the solution was diluted with ice-cold water and neutralized with 2 M NaOH until the pH of the solution rose between 5-6. The reaction mixture was extracted with EtOAc (4×75 mL). The combined organic layers were rinsed with brine and dried over anhydrous Na2SO₄. The solvent was removed using a rotary evaporator and the residue was purified by flash column chromatography with silica gel (50 g), using DCM/Hexane as eluent to give compound 90 (1.54 g, 76%).

### 6-bromoindoline (92)

Compound 91 (2 g, 10.2 mmol) was dissolved in acetic acid (5 mL). NaBH₃CN (1.92 g, 45.06 mmol) was added into the reaction flask portion-wise while maintaining the temperature below 10° C. The resulting solution was stirred for 1 h. After which, the solution was diluted with ice-cold water and neutralized with 2 M NaOH until the pH of the solution rose above 7. The reaction mixture was extracted with EtOAc (4×50 mL). The combined organic layers were rinsed with brine and dried over anhydrous Na2SO₄. The solvent was removed using a rotary evaporator and the residue was purified by flash column chromatography with silica gel (50 g), using DCM/Hexane as eluent to give compound 92 (1.60 g, 79%) as light brown oil.

### 6-iodoindoline (93)

Compound 93 was synthesized using a slightly modified protocol published by Klapars and Buchwald (J Amn Chem Soc 124, 14844-14845, doi:10.1021/ja028865v (2002)). An oven or flame dried, microwave glass tube was charged with a magnetic stir bar, compound 92 (1 g, 5.05 mmol), CuI (106 mg, 0.56 mmol), and NaI (1.66 g, 11.11 mmol). The glass tube was evacuated under vacuum and backfilled with N₂ 5 times. N,N′-dimethylethylenediamine (0.12 mL, 1.11 mmol) was added into the reaction vessel very quickly just before the tube was sealed with a Teflon cap. Anhydrous 1,4-dioxane (4.0 mL) was delivered via a syringe. The reaction was then heated to 110° C. and stirred for 24 h. After cooling to rt, the reaction mixture was diluted with 10 mL of saturated NH₄Cl and extracted with DCM (4×25 mL). The combined organic layers were washed with brine and dried over anhydrous Na₂SO₄, then concentrated in vacuo. The residue was purified by flash column chromatography with silica gel (25 g), using DCM/Hexane as eluent to give compound 93 (1.01 g, 82%) as a light brown solid.

### 6,6′-oxydiindoline (94)

Compound 94 was synthesized using a slightly modified protocol published by Maiti and Buchwald (J Am Chem Soc 131, 17423-17429, doi:10.1021/ja9081815 (2009)). An oven or flame dried, microwave glass tube was charged with a magnetic stir bar, compound 90 (150 mg, 1.11 mmol), 93 (272 mg, 1.11 mmol), CuI (21 mg, 0.11 mmol), 2-picolinic acid (27 mg, 0.22 mmol), and anhydrous K₃PO₄ (427 mg, 2.22 mmol). The glass tube was evacuated under vacuum and backfilled with N₂ 5 times before the tube was immediately sealed with a Teflon cap. Anhydrous DMSO (2 mL) was delivered via a syringe. The reaction was then heated to 85° C. and stirred for 18 h. After cooling to rt, the reaction mixture was diluted with 10 mL DI water and extracted with DCM (4×25 mL). The combined organic layers were washed with brine and dried over anhydrous Na₂SO₄, then concentrated in vacuo. The residue was purified by flash column chromatography with silica gel (25 g), using DCM/Hexane as eluent to give compound 94 (227 mg, 81%) as a colorless oil.

### (E)-6-(indolin-6-yloxy)-5-((4-nitrophenyl)diazenyl)indoline (95)

Compound 94 (90 mg, 0.36 mmol) was dissolved in 1 mL MeOH. The solution was chilled in an ice bath, then was treated with HCl (2 M, 5 mL). After 15 mins, p-nitrobenzenediazonium tetrafluoroborate (89 mg, 0.37 mmol) was added to the solution in 3 portions over 15 mins, then stirred at 0° C. for additional 1 h. During which time, the color of the reaction mixture changed from orange to dark red. After which, the solution was carefully basified with solid K₂CO₃ until pH value of the solution rose above 8. The deep red precipitate was collected via vacuum filtration and washed with small portions of DI water. The product was left in the funnel and air dried overnight to afford compound 95 (112 mg, 78%) as a red solid, which was used for the next step without further purification.

### 2,3,7,8-tetrahydro-1H-dipyrrolo[3,2-b:2′,3′-i]phenoxazin-9-ium (LGW-07-55)

Compound 95 (40 mg, 0.1 mmol) was dissolved in a solution of i-PrOH/H₂O (9/1, 2 mL) at 80° C. for 30 min, then the reaction mixture was treated with HClO₄ (70%, 10 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-07-55 (4.6 mg, 15%) as a dark blue solid.

LGW-07-59

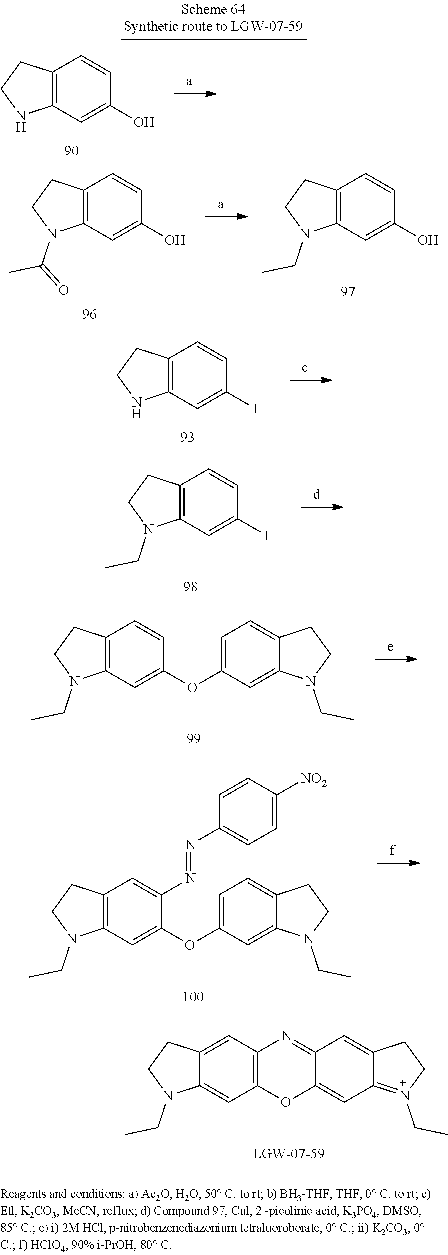

1-(6-hydroxyindolin-1-yl)ethan-1-one (96). Compound 90 (0.8 g, 5.92 mmol) was suspended in 10 mL DI water, to which Acetic anhydride (2.24 mL, 23.67 mmol) was added dropwise. The reaction mixture was placed in an ultrasonication bath for 1 min, then was stirred in a water bath (50° 0 C) for 10 min. The resulting solution was stirred overnight at rt. After which, the solid was collected via vacuum filtration and washed with small portions of DI water. The product was left in the funnel and air dried overnight to afford compound 96 (929 mg, 89%) as a light gray solid, which was used for the next step without further purification.

### 1-ethylindolin-6-ol (97)

A solution of 96 (0.9 g, 5.08 mmol) in anhydrous THF (15 mL) was stirred in an ice bath under N₂ for 30 mins. Borane tetrahydrofuran complex solution (1 M, 15 mL) was added to the solution above using a syringe pump over 30 mins, while maintaining the temperature of the solution below 5° C. The resulting reaction mixture was left in the ice bath and slowly warm to rt. After 24 h, the solution was placed in an ice bath again, and excess borane reagent was destroyed by carefully adding MeOH until no gas evolved. The solvent was evaporated under reduced pressure, the residue was purified by flash column chromatography with silica gel (25 g), using DCM/Hexane as eluent to give 97 (706 mg, 85%) as brown oil.

### 1-ethyl-6-iodoindoline (98)

To a suspension of compound 93 (0.4 g, 1.63 mmol) and K₂CO₃ (751 mg, 3.26 mmol) in anhydrous MeCN (10 mL) under N₂, was added Etl (0.16 mL, 1.96 mmol) at rt. The reaction mixture was then heated to reflux and stirred overnight. The solution was cooled down to rt and concentrated under reduced pressure. The crude product was diluted with DI water, and the resulting suspension was extracted with DCM (3×50 mL). The combined organic layers were rinsed with brine and dried over anhydrous Na₂SO₄. The solvent was removed using a rotary evaporator. The resulting residue was purified by flash column chromatography with silica gel (25 g), using DCM/Hexane as eluent to give compound 98 (369 mg, 83%) as clear oil.

### 6,6′-oxybis(1-ethylindoline) (99)

Compound 99 was synthesized using a slightly modified protocol published by Maiti and Buchwald (J Am Chem Soc 131, 17423-17429, doi:10.1021/ja9081815 (2009)). An oven or flame dried, microwave glass tube was charged with a magnetic stir bar, compound 97 (96 mg, 0.59 mmol), 98 (160 mg, 0.59 mmol), CuI (11 mg, 0.06 mmol), 2-picolinic acid (15 mg, 0.12 mmol), and anhydrous K₃PO₄ (249 mg, 1.17 mmol). The glass tube was evacuated under vacuum and backfilled with N₂ 5 times before the tube was immediately sealed with a Teflon cap. Anhydrous DMSO (1.5 mL) was delivered via a syringe. The reaction was then heated to 85° C. and stirred for 18 h. After cooling to rt, the reaction mixture was diluted with 10 mL DI water and extracted with DCM (4×25 mL). The combined organic layers were washed with brine and dried over anhydrous Na₂SO₄, then concentrated in vacuo. The residue was purified by flash column chromatography with silica gel (25 g), using DCM/Hexane as eluent to give compound 99 (151 mg, 84%) as a colorless oil.

### (E)-1-ethyl-6-((1-ethylindolin-6-yl)oxy)-5-((4-nitrophenyl)diazenyl)indoline (100)

Compound 99 (90 mg, 0.29 mmol) was dissolved in 1 mL MeOH. The solution was chilled in an ice bath, then was treated with HCl (2 M, 5 mL). After 15 mins, p-nitrobenzenediazonium tetrafluoroborate (73 mg, 0.31 mmol) was added to the solution in 3 portions over 15 mins, then stirred at 0° C. for an additional 1 h. During which time, the color of the reaction mixture changed from orange to dark red. After which, the solution was carefully basified with solid K₂CO₃ until the pH value of the solution rose above 8. The deep red precipitate was collected via vacuum filtration and washed with small portions of DI water. The product was left in the funnel and air dried overnight to afford compound 100 (109 mg, 82%), which was used for the next step without further purification.

### 1,9-diethyl-2,3,7,8-tetrahydro-1H-dipyrrolo[3,2-b:2′,3′-i]phenoxazin-9-ium (LGW-07-59)

Compound 100 (40 mg, 0.087 mmol) was dissolved in a solution of i-PrOH/H₂O (9/1, 2 mL) at 80° C. for 30 min, then the reaction mixture was treated with HClO₄ (70%, 10 μL). The resulting solution was stirred overnight. After which, the dark blue solution was evaporated under reduced pressure, and the residue was purified on a Biotage Isolera Flash System using SNAP Ultra cartridge with a mobile phase of CHCl₃ and MeOH containing 0.5% formic acid (gradient, 2-15% of MeOH in CHCl₃). The fractions containing product were pooled and evaporated, affording LGW-07-59 (17 mg, 60%) as a dark blue solid.

### Methods of Use

The formulations of the disclosure can be used to image nerves or nerve tissue. In particular embodiments, the formulations of the disclosure can be used to image nerves or nerve tissue in a subject. In particular embodiments, images of nerves can be obtained intraoperatively during FGS. In particular embodiments, the visualization of nerves during FGS allows surgery to be performed on tissue of interest while sparing nerves so as to reduce incidence of nerve injury during surgery. The area where surgery is performed or nearby regions can be surgically exposed. Surgery can be performed on organs, which include tissues such as nerve tissue, muscle tissue, and adipose tissue. The surgery can be laparoscopic, which is minimally invasive and includes the use of a thin, tubular device (laparoscope) that is inserted through a keyhole incision into a part of a subject's body, such as the abdomen or pelvis. The surgery can be assisted by a robot. Robot-assisted surgery can offer more precision, flexibility, and control, and is often associated with minimally invasive surgery.

A subject refers to any animal. The animal may be a mammal. Examples of suitable mammals include human and non-human primates, dogs, cats, sheep, cows, pigs, horses, mice, rats, rabbits, and guinea pigs.

A formulation of the disclosure can be directly applied to a tissue for imaging of nerves. In particular embodiments, direct application includes applying the formulation topically to a tissue to be imaged. In particular embodiments, direct application includes any route of application characterized by physical breaching of a tissue of a subject and application of the composition through the breach in the tissue. Direct application of a formulation includes application of a formulation to a tissue by injection, through a surgical incision, through a tissue-penetrating non-surgical wound, and the like. In particular embodiments, the tissue is undergoing surgery. In particular embodiments, direct application of a formulation includes applying the formulation to a surgical area, to nerves or nerve tissue, and to an exposed nerve. In particular embodiments, direct application of a formulation includes subcutaneous, intraperitoneal, or intramuscular application. In particular embodiments, a formulation can be delivered through a syringe or tubing to tissue. In particular embodiments, the tissue is ex vivo and is not undergoing surgery.

In particular embodiments, the fluorophore concentration in a formulation that is directly applied to nerve tissue includes a concentration range of 40 to 300 μg/mL. In particular embodiments, the fluorophore concentration in a formulation for direct application includes 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, and 200 μg/mL. In particular embodiments, the fluorophore concentration in a formulation for direct application is 50 μg/mL. In particular embodiments, the fluorophore concentration in a formulation for direct application is 200 μg/mL.

A formulation of the disclosure can be systemically applied to a subject for imaging of nerves. In particular embodiments, systemic application of a formulation includes intravenous injection of the formulation into a subject.

A formulation that is directly applied to a tissue can be allowed to penetrate the tissue for a given amount of time after direct application. In particular embodiments, the formulation can be allowed to penetrate the tissue for 30 seconds to 15 minutes, for 1 to 10 minutes, for 1 to 5 minutes, for 1 minute, for 2 minutes, for 3 minutes, for 4 minutes, or for 5 minutes. In particular embodiments, the formulation can be allowed to penetrate the tissue for 1 to 2 minutes. A formulation that is systemically applied to a subject can be administered a sufficient time before imaging such that the formulation can reach the area to be imaged and is present in such area at the time of imaging. In particular embodiments, a formulation that is systemically applied to a subject can be administered a sufficient time prior to imaging to allow uptake of the formulation by tissue in the subject. In particular embodiments, the formulation may be administered up to or less than 30 minutes, 1 hour, 2 hours, 3 hours, 4 hours, 5 hours, 6 hours, 7 hours, or 8 hours before imaging. The amount of time required may depend on the nerve imaging application and the administration site. In particular embodiments, the formulation is administered no more than 30 minutes, 1 hour, 2 hours, 3 hours, or 4 hours before imaging. In particular embodiments, the formulation is administered no more than 2 hours before imaging.

Tissue stained by a formulation including a fluorophore by direct application can be washed with buffer prior to imaging of the stained tissue. Washing of tissue stained by a formulation including a fluorophore can include flushing the tissue with an appropriate buffer and removing the buffer. In particular embodiments, the stained tissue can be washed 1 to 18 times, 1 to 10 times, 1 to 6 times, 1 time, 2 times, 3 times, 4 times, 5 times, or 6 times, with wash buffer. In particular embodiments, the stained tissue can be washed 6 times. In particular embodiments, the wash buffer is phosphate-buffered saline (PBS). In particular embodiments, washing the stained tissue removes unbound fluorophore. In particular embodiments, washing the stained tissue increases the nerve signal intensity and/or the signal to background ratio (SBR) as compared to no washing of the stained tissue. In particular embodiments, washing the stained tissue resolubilizes the fluorophore and allows for further diffusion of the fluorophore into the nerve tissue.

Imaging a tissue stained by a formulation including a fluorophore includes applying light to tissue that has been stained with a formulation of the disclosure. The light can be at a wavelength sufficient to excite the fluorophore in the formulation to fluoresce. In particular embodiments, light to excite the fluorophore is at a wavelength in the near infrared spectra. In particular embodiments, the fluorophore of a formulation emits at a wavelength in the near infrared spectra. In particular embodiments, the near infrared spectra includes a wavelength of 700 to 900 nm.

Imaging a tissue stained by a formulation including a fluorophore includes obtaining fluorescence images of the stained tissue by optical imaging systems such as ones described in the Examples.

In particular embodiments, imaging a tissue includes observing fluorescence images of the stained tissue. The fluorescence images can include still images (whether printed or on screen), or real-time images on a video monitor. In particular embodiments, the individual images of nerves obtained by staining of the nerves with the present formulations can be used for diagnostic purposes and for documentation of nerve location. By observing the fluorescence images the surgical team can determine the absence or presence of a nerve in the image. The surgical team can thus use information about the presence/absence or location of one or more nerves to determine how they will perform the surgical procedure. For example, based on information obtained through the disclosed methods, the surgical team may decide to perform a surgical cut at a point in the tissue where they are less likely to inadvertently cut or surgically contact a particular nerve based on the perceived absence of a nerve in an area of the tissue.

The information obtained from the obtained image can aid in grafting the ends of the nerves if they are transected. In the event of transection, nerve grafts can be applied directly to the ends to facilitate sprouting of regenerative neural fibers. In this case, the light visible from the fluorescence of the ends of transected nerves provides a target to guide the anastomosis of the nerves by the nerve graft.

Kits.

Formulations of the present disclosure to detect nerve tissue can also be provided as kits. Kits for detecting nerve tissue can include: a gel-based formulation including: (i) a fluorophore, and (ii) 5-10% sodium alginate and/or 18-26% PEO-PPO-PEO triblock copolymer; a formulation including (i) a fluorophore, and (ii) a DSPE-PEG micelle and/or cyclodextrin; and/or wash buffers. Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding: directly applying the formulations to a tissue; washing to remove excess formulation; systemically administering the formulations to a subject; applying light for visualization of the fluorophores; capturing fluorescent images of the tissue; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary laboratory and/or medical supplies needed to use the kit effectively, such as syringes, ampules, tubing, gloves, tubes, buffers, and the like. Variations in contents of any of the kits described herein can be made.

### Exemplary Embodiments

1. A gel-based formulation for tissue imaging including (i) a fluorophore, and (ii) 5-10% alginate salt and/or 18-26% PEO-PPO-PEO triblock copolymer.
2. The gel-based formulation of embodiment 1 wherein the fluorophore is an oxazine derivative.
3. The gel-based formulation of embodiment 2 wherein the oxazine derivative is LGW1-08.
4. The gel-based formulation of any one of embodiments 1-3 including 50 μg/mL fluorophore.
5. The gel-based formulation of any one of embodiments 1-3 including 200 μg/mL fluorophore.
6. The gel-based formulation of any one of embodiments 1-5 wherein the alginate salt is sodium alginate.
7. The gel-based formulation of embodiment 6 including 5-9% sodium alginate.
8. The gel-based formulation of embodiment 6 or 7 including 5-8% sodium alginate.
9. The gel-based formulation of any one of embodiments 6-8 including 6-8% sodium alginate.
10. The gel-based formulation of any one of embodiments 6-9 including 6-7% sodium alginate.
11. The gel-based formulation of any one of embodiments 6-10 including 6.5% sodium alginate.
12. The gel-based formulation of any one of embodiments 1-11 including 19-25% PEO-PPO-PEO triblock copolymer.
13. The gel-based formulation of any one of embodiments 1-12 including 20-24% PEO-PPO-PEO triblock copolymer.
14. The gel-based formulation of any one of embodiments 1-13 including 21-23% PEO-PPO-PEO triblock copolymer.
15. The gel-based formulation of any one of embodiments 1-14 including 22% PEO-PPO-PEO triblock copolymer.
16. The gel-based formulation of any one of embodiments 6-13 including 5-8% sodium alginate and/or 20-24% PEO-PPO-PEO block copolymer.
17. The gel-based formulation of any one of embodiments 10-13 including 6-7% sodium alginate and/or 20-24% PEO-PPO-PEO block copolymer.
18. The gel-based formulation of any one of embodiments 10-14 including 6-7% sodium alginate and/or 21-23% PEO-PPO-PEO block copolymer.
19. A method of directly applying the gel-based formulation of any one of embodiments 1-18 including applying the gel-based formulation to an exposed nerve.
20. The method of embodiment 19 wherein the applying is during radical prostatectomy.
21. The method of embodiment 19 or 20 further including washing the applied gel-based formulation from the nerve.
22. The method of any one of embodiments 19-21 wherein the washing includes 5-7 flushes.
23. A formulation for systemic administration tissue imaging including (i) a fluorophore, and (ii) a DSPE-PEG micelle and/or cyclodextrin.
24. The formulation of embodiment 23 wherein the fluorophore is an oxazine derivative.
25. The formulation of embodiment 24 wherein the oxazine derivative is LGW1-08.
26. The formulation of any one of embodiments 23-25 including a DSPE-PEG micelle with the fluorophore encapsulated at 0.5-0.9 mg/mL.
27. The formulation of any one of embodiments 23-26 including a DSPE-PEG micelle with the fluorophore encapsulated at 0.7 mg/mL.
28. The formulation of any one of embodiments 23-25 including cyclodextrin with the fluorophore encapsulated at 0.5-1.2 mg/mL.
29. The formulation of any one of embodiments 23-25 or 28 including cyclodextrin with the fluorophore encapsulated at 0.7-1.0 mg/mL.
30. A method of staining a nerve or tissue including systemically administering a formulation of any of embodiments 23-29 to a subject during an operative procedure.
31. The method of embodiment 30 wherein the administering is at dose of 2.5 mg/kg.
32. A method of detecting nerves intraoperatively in a subject undergoing surgery including:

directly applying a gel-based formulation including (i) a fluorophore, and (ii) 5-10% alginate salt and/or 18-26% PEO-PPO-PEO triblock copolymer to stain tissue undergoing surgery; and

imaging the stained tissue, thereby detecting nerves intraoperatively in the subject undergoing surgery.

33. The method of embodiment 32, further including washing the tissue with buffer after applying the gel-based formulation and prior to imaging the stained tissue.
34. The method of embodiment 33, wherein the washing removes unbound fluorophore.
35. The method of embodiment 33 or 34, wherein the buffer is phosphate-buffered saline (PBS).
36. The method of any one of embodiments 33-35, further including allowing the gel-based formulation to penetrate the tissue for 30 seconds to 5 minutes prior to the washing.
37. The method of any one of embodiments 33-36, further including allowing the gel-based formulation to penetrate the tissue for 1 minute to 2 minutes prior to the washing.
38. The method of any one of embodiments 32-37, wherein risk of iatrogenic injury to the subject undergoing surgery is reduced.
39. The method of any one of embodiments 32-38, wherein the surgery is laparoscopic.
40. The method of any one of embodiments 32-39, wherein the surgery is performed by a robot.
41. The method of any one of embodiments 32-40, wherein the surgery is radical prostatectomy.
42. The method of any one of embodiments 32-41, wherein the fluorophore is an oxazine derivative.
43. The method of any one of embodiments 32-42, wherein the oxazine derivative is LGW1-08.
44. The method of any one of embodiments 32-43, wherein the concentration of the fluorophore is 50 μg/mL.
45. The method of any one of embodiments 32-43, wherein the concentration of the fluorophore is 200 μg/mL.
46. The method of any one of embodiments 32-45 wherein the alginate salt is sodium alginate.
47. The method of embodiment 46 including 5-9% sodium alginate.
48. The method of embodiment 46 or 47 including 5-8% sodium alginate.
49. The method of any one of embodiments 46-48 including 6-8% sodium alginate.
50. The method of any one of embodiments 46-49 including 6-7% sodium alginate.
51. The method of any one of embodiments 46-50 including 6.5% sodium alginate.
52. The method of any one of embodiments 46-51 including 19-25% PEO-PPO-PEO triblock copolymer.
53. The method of any one of embodiments 46-52 including 20-24% PEO-PPO-PEO triblock copolymer.
54. The method of any one of embodiments 46-53 including 21-23% PEO-PPO-PEO triblock copolymer.
55. The method of any one of embodiments 46-54 including 22% PEO-PPO-PEO triblock copolymer.
56. The method of any one of embodiments 46-53 including 5-8% sodium alginate and/or 20-24% PEO-PPO-PEO block copolymer.
57. The method of any one of embodiments 50-53 including 6-7% sodium alginate and/or 20-24% PEO-PPO-PEO block copolymer.
58. The method of any one of embodiments 50-54 including 6-7% sodium alginate and/or 21-23% PEO-PPO-PEO block copolymer.
59. A method of detecting nerves within ex vivo tissue including:

directly applying a gel-based formulation including (i) a fluorophore, and (ii) 5-10% alginate salt and/or 18-26% PEO-PPO-PEO triblock copolymer to stain the ex vivo tissue; and

imaging the stained ex vivo tissue, thereby detecting nerves within the ex vivo tissue.

60. The method of embodiment 59, further including washing the ex vivo tissue with buffer after applying the gel-based formulation and prior to imaging the stained ex vivo tissue.
61. The method of embodiment 59 or 60, wherein the washing removes unbound fluorophore.
62. The method of embodiment 60 or 61, wherein the buffer is phosphate-buffered saline (PBS).
63. The method of any one of embodiments 60-62, further including allowing the gel-based formulation to penetrate the tissue for 30 seconds to 5 minutes prior to the washing.
64. The method of any one of embodiments 60-63, further including allowing the gel-based formulation to penetrate the tissue for 1 minute to 2 minutes prior to the washing.
65. The method of any one of embodiments 59-64, wherein the fluorophore is an oxazine derivative.
66. The method of any one of embodiments 59-65, wherein the oxazine derivative is LGW1-08
67. The method of any one of embodiments 59-66, wherein the concentration of the fluorophore is 50 μg/mL.
68. The method of any one of embodiments 59-66, wherein the concentration of the fluorophore is 200 μg/mL.
69. The method of any one of embodiments 59-68 wherein the alginate salt is sodium alginate.
70. The method of embodiment 69 including 5-9% sodium alginate.
71. The method of embodiment 69 or 70 including 5-8% sodium alginate.
72. The method of any one of embodiments 69-71 including 6-8% sodium alginate.
73. The method of any one of embodiments 69-72 including 6-7% sodium alginate.
74. The method of any one of embodiments 69-73 including 6.5% sodium alginate.
75. The method of any one of embodiments 69-74 including 19-25% PEO-PPO-PEO triblock copolymer.
76. The method of any one of embodiments 69-75 including 20-24% PEO-PPO-PEO triblock copolymer.
77. The method of any one of embodiments 69-76 including 21-23% PEO-PPO-PEO triblock copolymer.
78. The method of any one of embodiments 69-77 including 22% PEO-PPO-PEO triblock copolymer.
79. The method of any one of embodiments 69-76 including 5-8% sodium alginate and/or 20-24% PEO-PPO-PEO block copolymer.
80. The method of any one of embodiments 73-76 including 6-7% sodium alginate and/or 20-24% PEO-PPO-PEO block copolymer.
81. The method of any one of embodiments 73-77 including 6-7% sodium alginate and/or 21-23% PEO-PPO-PEO block copolymer.
82. A method of detecting nerves intraoperatively in a subject undergoing surgery including:

systemically administering a formulation (i) a fluorophore, and (ii) a DSPE-PEG micelle and/or cyclodextrin to the subject before or during surgery; and

imaging stained tissue undergoing surgery in the subject, thereby detecting nerves intraoperatively in the subject undergoing surgery.

83. The method of embodiment 82, wherein systemically administering includes intravenously injecting the subject with the formulation.
84. The method of embodiment 82 or 83, including systemically administering the formulation 30 minutes to 4 hours prior to the imaging.
85. The method of embodiment 82 or 83, including systemically administering the formulation 2 hours prior to the imaging.
86. The method of any one of embodiments 82-85, wherein risk of iatrogenic injury to the subject undergoing surgery is reduced.
87. The method of any one of embodiments 82-86, wherein the surgery is laparoscopic.
88. The method of any one of embodiments 82-87, wherein the surgery is performed by a robot.
89. The method of any one of embodiments 82-88, wherein the fluorophore is an oxazine derivative.
90. The method of any one of embodiments 82-89, wherein the oxazine derivative is LGW01-08.
91. The method of any one of embodiments 82-90 including a DSPE-PEG micelle with the fluorophore encapsulated at 0.5-0.9 mg/mL.
92. The method of any one of embodiments 82-91 including a DSPE-PEG micelle with the fluorophore encapsulated at 0.7 mg/mL.
93. The method of any one of embodiments 82-90 including cyclodextrin with the fluorophore encapsulated at 0.5-1.2 mg/mL.
94. The method of any one of embodiments 82-90 or 93 including cyclodextrin with the fluorophore encapsulated at 0.7-1.0 mg/mL.
95. A kit including:

(a) a gel-based formulation including (i) a fluorophore, and (ii) 5-10% alginate salt and/or 18-26% PEO-PPO-PEO triblock copolymer; and/or

(b) a formulation including (i) a fluorophore, and (ii) a DSPE-PEG micelle and/or cyclodextrin; and

(c) use instructions for applying the formulation of (a) and/or administering the formulation of (b).

96. The kit of embodiment 95, wherein the fluorophore is an oxazine derivative.
97. The kit of embodiment 96, wherein the oxazine derivative is LGW1-08.
98. The kit of any one of embodiments 95-97, wherein the concentration of the fluorophore is 50 μg/mL.
99. The kit of any one of embodiments 95-97, wherein the concentration of the fluorophore is 200 μg/mL.
100. The kit of any one of embodiments 95-99 wherein the alginate salt is sodium alginate.
101. The kit of embodiment 100 including 5-9% sodium alginate.
102. The kit of embodiment 100 or 101 including 5-8% sodium alginate.
103. The kit of any one of embodiments 100-102 including 6-8% sodium alginate.
104. The kit of any one of embodiments 100-103 including 6-7% sodium alginate.
105. The kit of any one of embodiments 100-104 including 6.5% sodium alginate.
106. The kit of any one of embodiments 95-105 including 19-25% PEO-PPO-PEO triblock copolymer.
107. The kit of any one of embodiments 95-106 including 20-24% PEO-PPO-PEO triblock copolymer.
108. The kit of any one of embodiments 95-107 including 21-23% PEO-PPO-PEO triblock copolymer.
109. The kit of any one of embodiments 95-108 including 22% PEO-PPO-PEO triblock copolymer.
110. The kit of any one of embodiments 100-107 including 5-8% sodium alginate and/or 20-24% PEO-PPO-PEO block copolymer.
111. The kit of any one of embodiments 104-107 including 6-7% sodium alginate and/or 20-24% PEO-PPO-PEO block copolymer.
112. The kit of any one of embodiments 104-108 including 6-7% sodium alginate and/or 21-23% PEO-PPO-PEO block copolymer.
113. The kit of any one of embodiments 95-112 including a DSPE-PEG micelle with the fluorophore encapsulated at 0.5-0.9 mg/mL.
114. The kit of any one of embodiments 95-113 including a DSPE-PEG micelle with the fluorophore encapsulated at 0.7 mg/mL.
115. The kit of any one of embodiments 95-112 including cyclodextrin with the fluorophore encapsulated at 0.5-1.2 mg/mL.
116. The kit of any one of embodiments 95-112 or 115 including cyclodextrin with the fluorophore encapsulated at 0.7-1.0 mg/mL.

### Example 1. Improved Formulations for Direct Administration of Nerve Specific Probes for Fluorescence Image Guided Surgery

This example provides clinically viable formulations of a novel near-infrared nerve specific oxazine fluorophore, LGW1-08, that possess unique gelling characteristics. The formulations are useful for direct (local) administration to a subject, allowing an excellent platform for clinical translation of nerve-specific fluorophores for fluorescence guided surgery (FGS).

Iatrogenic nerve injury significantly affects surgical outcomes for procedures like the radical prostatectomy (RP), with up to 60% of patients reporting nerve damage one to two years post-surgery. Although nerve sparing RP techniques have been practices for over 30 years, it remains difficult for surgeons to identify nerve tissue intraoperatively and nerve sparing success rates are strongly correlated with experience level. Fluorescence guided surgery (FGS) offers a potential solution for improved nerve sparing by providing direct visualization of nerve tissue intraoperatively. However, novel probes for FGS face an extraordinary regulatory challenge to achieve clinical translation. Previously, a direct administration methodology was developed that enabled application of nerve-specific fluorophores at a much lower dose than systemic administration for clinical translation via exploratory IND guidance. However, a clinically viable formulation was necessary to advance this promising technology to clinical use. Previously a non-FDA approved co-solvent formulation was utilized which resulted in significant background staining in preliminary large animal studies from a lack of staining control inherent to liquid-based formulations. In the present study, we report on the development of a clinically viable gel-based formulation strategy that enables direct administration of a nerve-specific fluorescent contrast agent with increased control for nerve sparing FGS applications. An F127 Pluronic® formulation was used to solubilize a novel near-infrared nerve specific oxazine fluorophore, LGW1-08, providing increased staining control for a variety of tissue surfaces, angles, and morphologies. Additionally, the formulation developed herein possesses unique gelling characteristics, allowing it to easily be spread as a liquid followed by rapid gelling at body temperature for subsequent tissue hold. Further optimization of the direct administration protocol has decreased the total staining time to 1-2 minutes, improving compatibility with surgical procedures. The resulting gel formulation and direct administration methodology provides an excellent platform for clinical translation of novel nerve-specific fluorophores for FGS.

Materials and Methods.

Contrast agents and formulations. LGW01-08 was chosen as the lead compound for development from a library of 64 oxazine derivatives. The previously used co-solvent formulation containing 10% dimethyl sulfoxide (DMSO), 5% Kolliphor EL, 65% serum, and 20% phosphate buffered saline (PBS) was used for comparison to the gel formulations presented herein (Gibbs-Strauss et al. Molecular imaging 10, 91-101 (2011)). PLURONIC®/poloxamers are FDA approved poly(ethylene oxide)/poly(propylene oxide)/poly(ethylene oxide) (PEO-PPO-PEO) triblock copolymers, aqueous solutions of which undergo sol-to-gel transition with increasing the temperature above a lower critical gelation temperature. Nie et al. Int J Nanomedicine 6, 151-166 (2011); Diniz et al. J Mater Sci Mater Med 26, 153 (2015); Suntornnond et al. Sci Rep 7, 16902 (2017). PLURONIC®127 NF (Letco medical, Decatur, Ala.) was added to cold Millipore purified water to achieve 20, 22, and 25% F127 gel concentration. The solutions were left overnight on a shaker in the cold room, to ensure complete solubilization of the polymer. Alginates have been used as versatile bio-polymers for numerous applications. They have a unique property of gelling in presences of divalent ions such as calcium. 5.0%, 6.8% and 8% alginate gels were prepared by dissolving sodium alginate (Sigma Aldrich, St. Louis, Mo.) in hot water for 2-3 h on a shaker to ensure complete wetting and eventually solubilization of alginate.

Animals.

Approval for the use of all small animals in this study was obtained from the controlling Institutional Animal Care and Use Committee (IACUC). Male CD-1 mice weighing 22-24 g were purchased from Charles River Laboratories (Wilmington, Mass.). Prior to surgery, animals were anaesthetized with 100 mg/kg ketamine and 10 mg/kg xylazine (Patterson Veterinary, Devens, Mass.) administered intraperitoneally (IP). The brachial plexus and sciatic nerves were surgically exposed by removal of overlaying adipose and muscle tissues for direct nerve staining and imaging.

Gel formulation screening and viscosity testing. Each gel-based formulation was tested in mice using the disclosed direct administration methodology developed using the liquid based co-solvent formulation to ensure no significant loss in the resulting fluorescence intensity or nerve signal-to-background ratios. Barth & Gibbs. Theranostics 7, 573-593 (2017). LGW01-08 was formulated in 5, 6.5, and 8% Na Alginate as well as 20, 22, and 25% PEO-PPO-PEO triblock (Pluronics®) at 125 μM concentration. Each exposed brachial plexus and sciatic nerve site was incubated with the formulated fluorophore for 5 min, followed by 9 flushes with PBS, a secondary 5 min incubation with blank formulation, and 9 additional washes to remove non-specifically bound fluorophore. Fluorescence and color images were acquired immediately following staining and 30 minutes following staining to observe improvements in nerve SBRs due to clearance. Nerve sites were stained with LGW01-08 in the previously utilized co-solvent formulation as positive controls and unstained nerve sites were imaged as negative controls (n=3 mice or 6 nerve sites per condition) (Gibbs-Strauss et al. Molecular imaging 10, 91-101 (2011); Gibbs et al. Structure-activity relationship of nerve-highlighting fluorophores. PloS one 8, e73493 (2013); Barth & Gibbs. Theranostics 7, 573-593 (2017)).

To determine the most clinically viable amount of viscosity enhancer within each gel formulation, Oxazine 4 at 1 mM in 5, 6.5, and 8% Na Alginate as well as 20, 22, and 25% PEO-PPO-PEO triblock was dropped onto swine intestinal tissue that had been shimmed to 0, 35, and 65° angles. A 1 mL pipette was used to apply 300 μL of each formulation to porcine small intestine held on an angled shim. The small intestine was kept at internal body temperature and only removed externally for testing the formulation. A new section of small intestine was used to test each local formulation. All small intestine regions were gently wiped with medical gauze to remove any noticeable quantities of serous fluid. A color photograph was taken 30 seconds after local formulation application for surface area analysis using ruler measurements on the tissue shims adjacent to the tissue. All local formulations were tested in triplicate. Surface area measurements were used to determine the tissue spread and administration control characteristics.

Direct Administration Protocol Testing.

To adapt the previously developed direct administration methodology to gel formulation application, the fluorophore concentration, incubation time, and washing protocols were analyzed for the optimal gel formulations. 25, 50, and 200 μg/mL concentrations of LGW01-08 at 1 and 5 min incubation times in 22% PEO-PPO-PEO triblock were used to stain the brachial plexus and sciatic nerve sites followed by 18 PBS wash steps. Following determination of the most clinically viable concentration and incubation time, efforts to shorten the washing protocol were investigated. Images were collected following each flush step for 6 washes and then every 3 washes out to 18 washes. Washing was completed using PBS stored at 4° C. to test for improved ability to liquify the gelled PEO-PPO-PEO triblock solution compared to warm 37° C. PBS. Washes consisted of a short flush of PBS followed immediately by removal by absorption with gauze.

Toxicity Testing.

Two cohorts of mice per formulation were subcutaneously administered 0.5 mL 22% PEO-PPO-PEO triblock loaded with LGW1-08 for a dose of 1.8 mg/kg and unloaded 22% PEO-PPO-PEO triblock for blood chemistry analysis and 14-day weight monitoring (n=5 mice per cohort per formulation). The dose was calculated based on body surface area scaling between mice and humans (12.3 scaling factor) to be 100× the maximum dose allowed for microdose studies (100 μg). Blood chemistry cohort mice were euthanized 24 hours following systemic administration and blood was collected into lithium heparin tubes via cardiac puncture. Blood was sent to IDEXX laboratory (Veterinary Diagnostic, Portland, Oreg.) for standard blood chemistry analysis. The blood markers evaluated include blood urea nitrogen (BUN), creatinine kinase (CK), alanine transaminase (ALT), aspartate transaminase (AST), and alkaline phosphatase (ALP). Hematological analysis involved assessment of white blood cell (WBC) count, red blood cell (RBC) count, Hematocrit (HC), % neutrophils, % lymphocyte, % monocyte, % eosinophil, and % basophile. The electrolytes assessed include phosphorus, calcium, sodium, potassium, and chloride. Weight monitoring cohort mice were weighed 1, 4, 6, 8, 10, 11, and 14 days following administration.

Large Animal Testing.

To validate the direct administration protocol and gel formulations in a more clinically relevant surgical model, the gel formulation was applied laparoscopically to the iliac plexus in swine using the da Vinci Si surgical robotic system (Intuitive Surgical, Sunnyvale, Calif.). The direct administration gel and liquid formulation protocols were used to stain swine iliac plexus and the surrounding muscle and adipose tissue with 1 mM Oxazine 4 solubilized in the PEO-PPO-PEO triblock and cosolvent formulations, respectively. Color and fluorescence images were acquired two and a half hours following staining to ensure adequate nerve contrast due to clearance of the higher dose administered and that resulting nerve signal was maintained for the duration of a typical surgical procedure.

Intraoperative fluorescence imaging system. A custom-built small animal imaging system capable of real-time color and fluorescence imaging was used to acquire rodent in vivo images. The imaging system consisted of a QImaging EXi Blue monochrome camera (Surrey, British Columbia, CA) for fluorescence detection with a removable Bayer filter for collecting co-registered color and fluorescence images. A PhotoFluor II light source (89 North, Burlington, Vt.) was focused onto the surgical field through a liquid light guide and used unfiltered for white light illumination. For fluorescence excitation, the PhotoFluor II was filtered with a 620±30 nm bandpass excitation filter. Resulting fluorescence was collected with a 700±37.5 nm bandpass emission filter for image collection. All filters were obtained from Chroma Technology (Bellows Falls, Vt.). Camera exposure times ranged from 10-2000 ms for fluorescence image collection.

A custom-built laparoscopic imaging system also capable of real-time color and fluorescence imaging was used to acquire swine in vivo images. The imaging system was integrated into the da Vinci Si surgical system from Intuitive Surgical, Inc. (Sunnyvale, Calif.) and consisted of a Necsel Neon 5 W 640 nm laser (Necsel, Milpitas, Calif.) coupled to the da Vinci Si endoscope with a Semrock 642 nm StopLine single-notch blocking filter (Semrock, Rochester N.Y.) to remove excitation light from the acquired fluorescence image. The 642 nm StopLine blocking filter was placed in the Si camera sterile adaptor between the rod lens endoscope and the Si camera head. Fluorescent signal acquisition occurred in the Si white light mode with the blocking filter removing the 642 nm excitation light and the fluorescent signal detected primarily on the RGB red-Bayer elements. Laser power at the endoscope tip measured 800 mW with optical power loses occurring primarily at the laser fiber/light guide and the Si camera head/rod lens interfaces. Fluorescence and color videos were captured using the Si Vision Side Cart at an exposure time of 2 ms. Images were taken from screen captures of the in vivo video clips. Videos were recorded on a Panasonic SDI recorder connected to the TilePro video out connections on da Vinci Si Vision Side Cart.

Intraoperative Nerve Imaging and Image Analysis.

Nerve specific contrast was assessed for all initial testing, method analysis, and large animal studies using the intraoperative fluorescence imaging systems. Additional unstained control animals were imaged to assess tissue autofluorescence.

Custom written MatLab code was used to analyze the tissue specific fluorescence where regions of interest were selected using the white light images. These regions of interest were then analyzed on the co-registered matched fluorescence images permitting assessment of the mean tissue intensities and nerve to muscle (N/M), nerve to adipose (N/A), and nerve to cut muscle (N/CM) ratios. Intensity measurements were divided by the exposure time to obtain normalized intensity per second measurements.

Statistical Analysis.

Significant differences between nerve SBR means were evaluated using a one-way ANOVA followed by a Fisher's LSD multiple comparison test with no assumption of sphericity using the Geisser-Greenhouse correction to compare all mean nerve signal intensities and nerve to background tissue ratios. The a value was set to 0.05 for all analyses. Results were presented as mean±standard deviation (S.D.). All statistical analysis was performed using GraphPad Prism (La Jolla, Calif.).

Results.

Preliminary Nerve Specificity Screening Using Gel-Based Formulations.

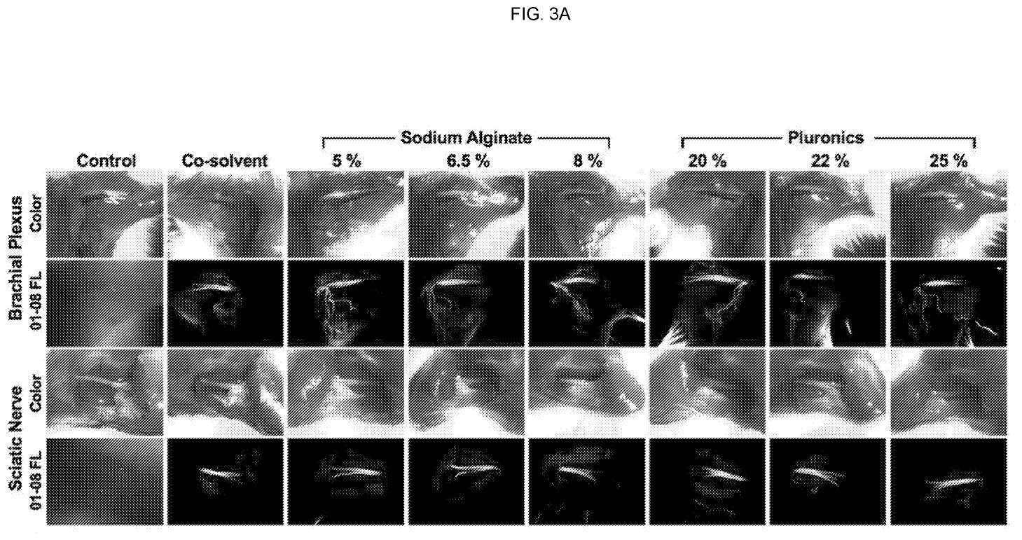

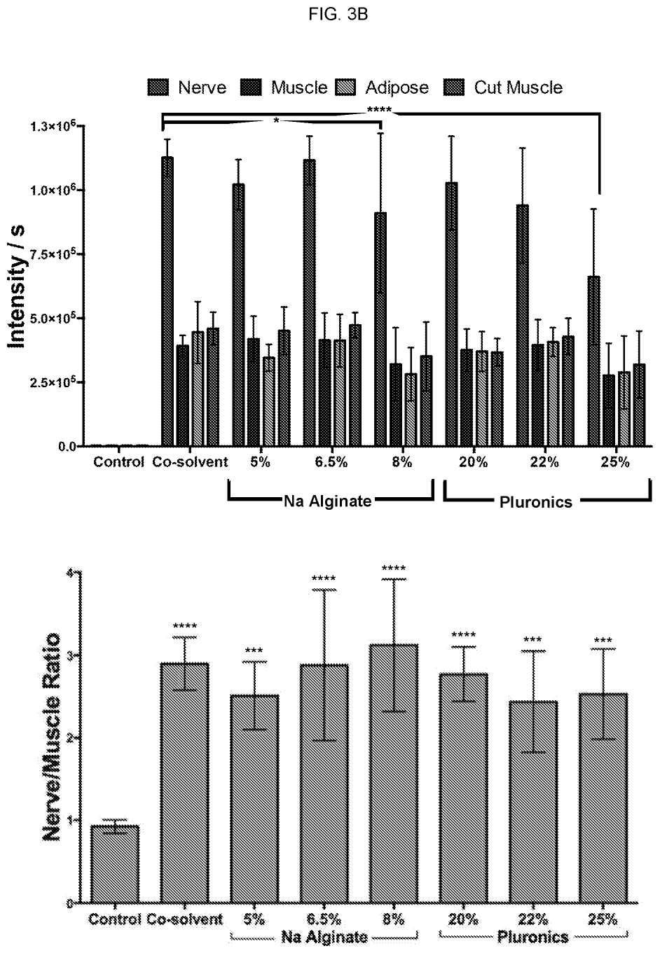

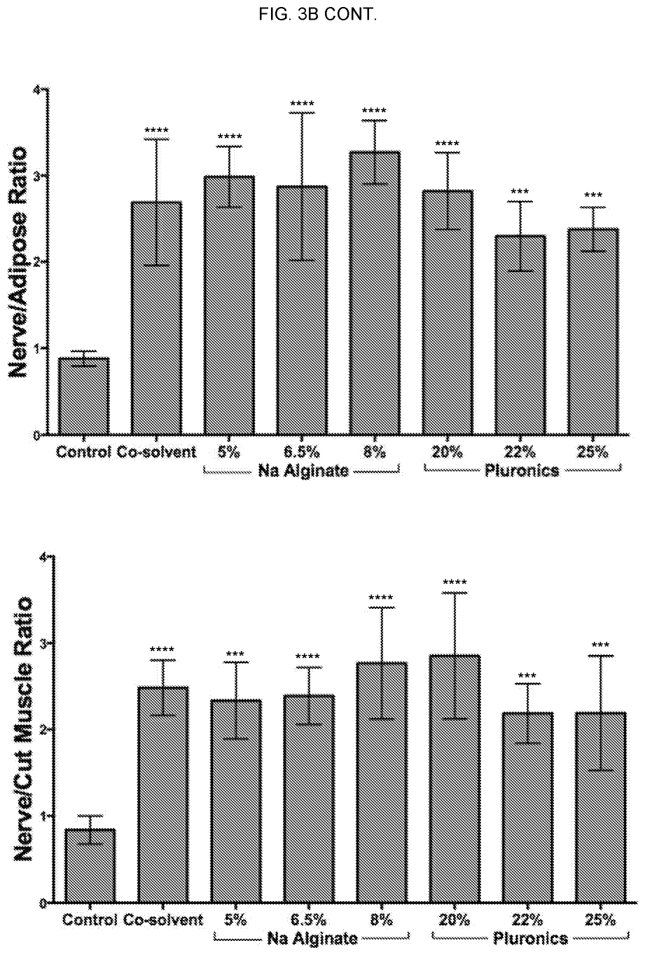

Two viscosity enhancers, sodium alginate (Na Alginate) and PEO-PPO-PEO triblock, were used to create gel formulations for administration of the nerve specific fluorophore LGW01-08. Mouse brachial plexus and sciatic nerves were stained using direct administration with varying concentrations of each viscosity enhancer (5, 6.5, and 8% Na Alginate; 20, 22, and 25% PEO-PPO-PEO triblock) to solubilize 125 μM LGW01-08. The staining results were compared to the previously utilized liquid based co-solvent formulation containing 125 μM LGW01-08 (FIGS. 3A, 3B). No major differences in nerve highlighting ability were observed for all concentrations of Na Alginate or PEO-PPO-PEO triblock compared to the co-solvent formulation (FIG. 3A). No significant difference in nerve SBRs were observed between all formulations, and all stain groups had significantly higher N/M, N/A, and N/CM ratios compared to the control unstained group. Additionally, aside from the highest concentrations of each viscosity enhancer (8% Na Alginate and 25% PEO-PPO-PEO triblock) all gel formulations displayed similar nerve fluorescence intensities to the co-solvent formulation (FIG. 3B).

Formulation Composition, Stability, and Clinical Status.

The formulations tested herein were chosen for their high stability, clinical approval status, and advantageous viscosity and penetration characteristics. The composition, stability, and clinical toxicity profiles are outlined in Table 1. Both Na Alginate and PEO-PPO-PEO triblock formulations provide unique and beneficial gel forming characteristics, while the co-solvent formulation is liquid only. Additionally, Na Alginate and PEO-PPO-PEO triblock formulations are FDA approved, providing clinically viable formulation candidates.

TABLE 1
Gel formulation composition, gel characteristics, and regulatory status.

| Formulation | Composition | Gel forming characteristics | Regulatory status |
|---|---|---|---|
| Cosolvent | 10% DMSO, 5% | Liquid only | None |
| | Kolliphor, 85% 75/25 | | |
| | Serum/Buffer | | |
| Na Alginate | Sodium Alginate | Liquid to solid transition as | FDA Approved |
| | | temperature decreases, can form | |
| | | hydrogel in presence of | |
| | | Ca⁺² ions in tissue | |
| PEO-PPO- | PEO-PPO-PEO | Liquid to solid transition as | FDA Approved |
| PEO triblock | triblock copolymers (F- | temperature increases over a | |
| | 127 PLURONIC ®) | critical threshold, gel formation | |
| | | is reversible | |

Gel Formulation Viscosity Assessment.

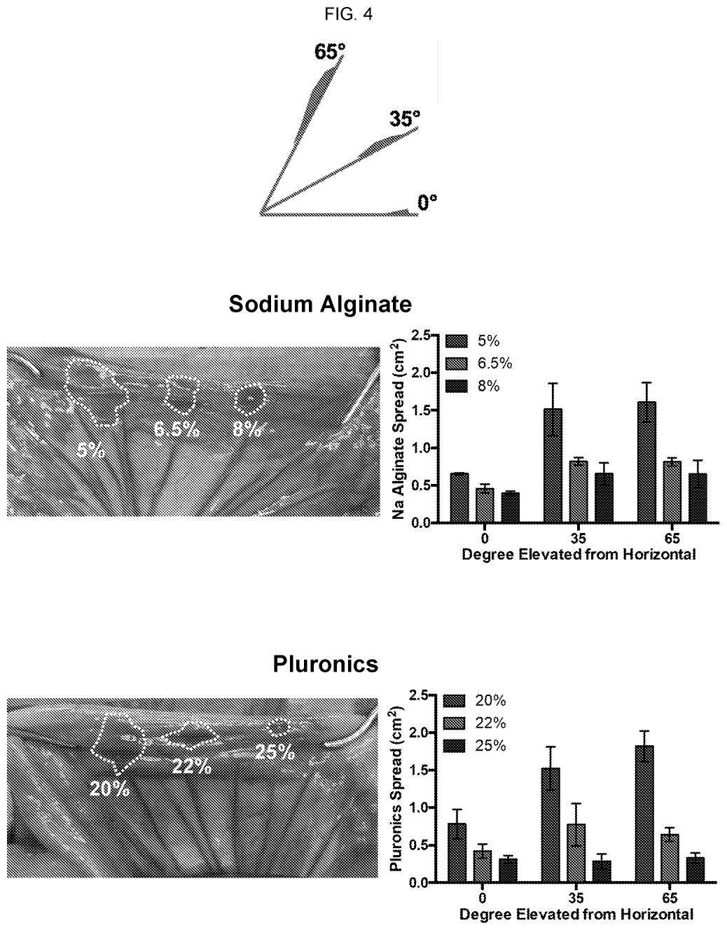

To assess the clinical utility of the viscosity enhancers for each gel formulation, tissue spread in swine was determined using formulations with 5, 6.5, and 8% Na Alginate as well as 20, 22, and 25% PEO-PPO-PEO triblock at varying degrees of tilt (0°, 35°, and 65°) (FIG. 4). The lowest concentration of each viscosity enhancer tested (5% Na Alginate and 20% PEO-PPO-PEO triblock) provided little resistance to increased tilt angle and saw significant increases in the measured spread surface area at 35° and 65° tilts. The middle concentration of each viscosity enhancer tested (6.5% Na Alginate and 22% PEO-PPO-PEO triblock) provided adequate hold and did not see a large increase in spread at higher degrees of tilt. The highest concentration of each viscosity enhancer tested (8% Na Alginate and 25% PEO-PPO-PEO triblock) allowed only minimal initial spread and no changes in tissue spread at higher tilt angles (FIG. 4). Due to the tradeoff between ability to spread following initial application and subsequent hold at more vertical surface angles, the middle concentrations (6.5% Na Alginate and 22% PEO-PPO-PEO triblock) were chosen as most clinically relevant for each formulation. Additionally, PEO-PPO-PEO triblock was found to have more favorable gelling characteristics compared to Na Alginate. The viscosity of PEO-PPO-PEO triblock increases at body temperature and decreases at cooler temperatures, including room temperature facilitating application to a tissue surface. By comparison, Na Alginate increases viscosity at cooler temperatures and decreases viscosity at higher temperatures, making both application and tissue gelling less advantageous. Due to these characteristics, PEO-PPO-PEO triblock was chosen as the final viscosity enhancer for the gel formulation since it allowed initial spreading in the tissue to occur with relative ease and subsequently gelled quickly, remaining at the site of application on the tissues in the body cavity.

Direct Administration Method Testing.

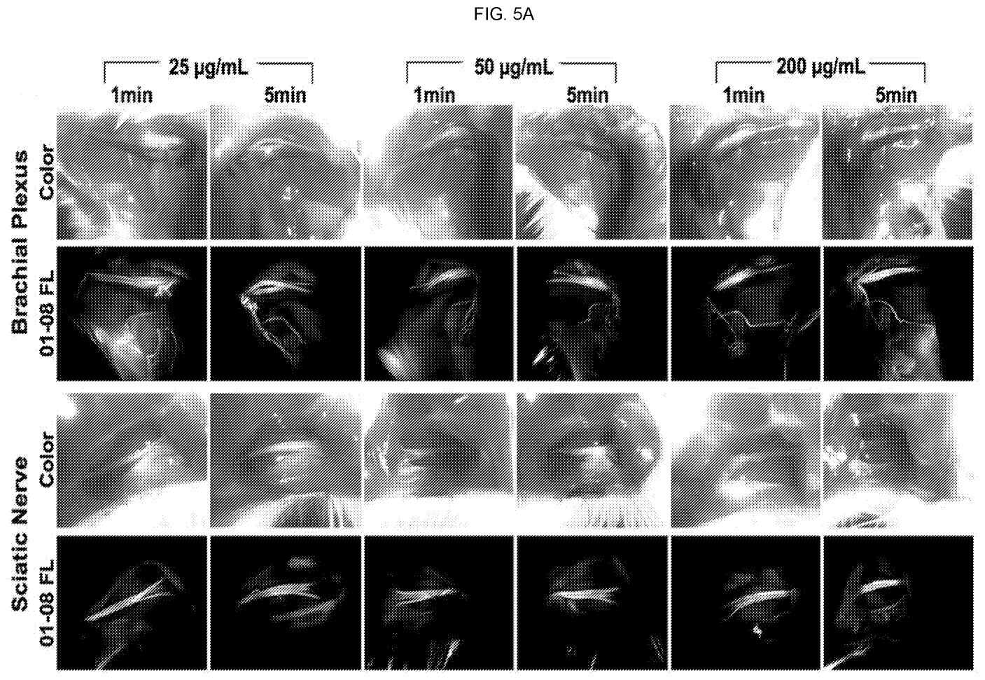

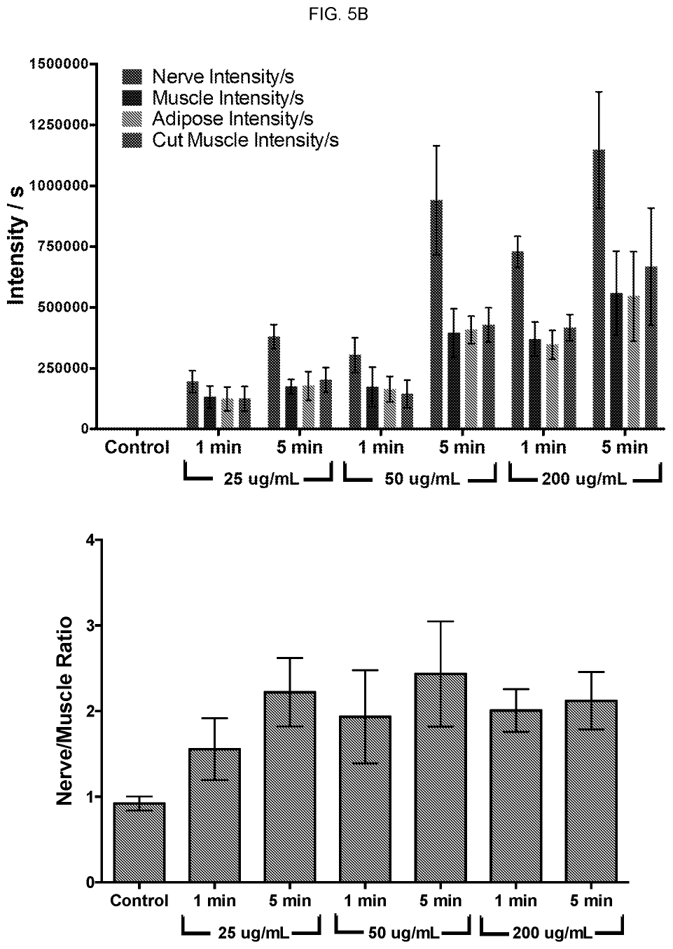

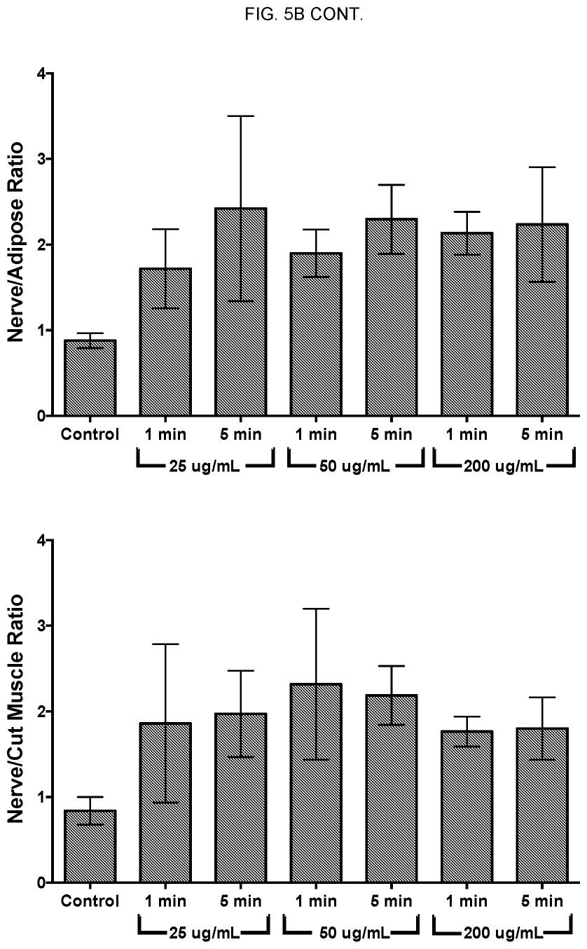

Using the chosen gel formulation, the direct administration protocol was optimized to improve gel formulation staining application. First, several fluorophore concentration and incubation time parameters were tested to determine a clinically viable staining solution and method (FIGS. 5A, 5B). Nerve contrast remained consistent across concentrations and incubation times tested in brachial plexus and sciatic nerve models (FIG. 5A). Additionally, nerve SBR values remained relatively consistent across all concentrations and incubation times. However, fluorescence signal intensities decreased at lower concentrations and incubation times (FIG. 5B). For this reason, the highest concentration tested, 200 μg/mL, which is the highest concentration that when scaled to humans still falls within the microdosing range for eIND studies, was chosen for further studies. In order to minimize the time needed to perform the staining protocol, the one-minute incubation time was chosen for further studies.

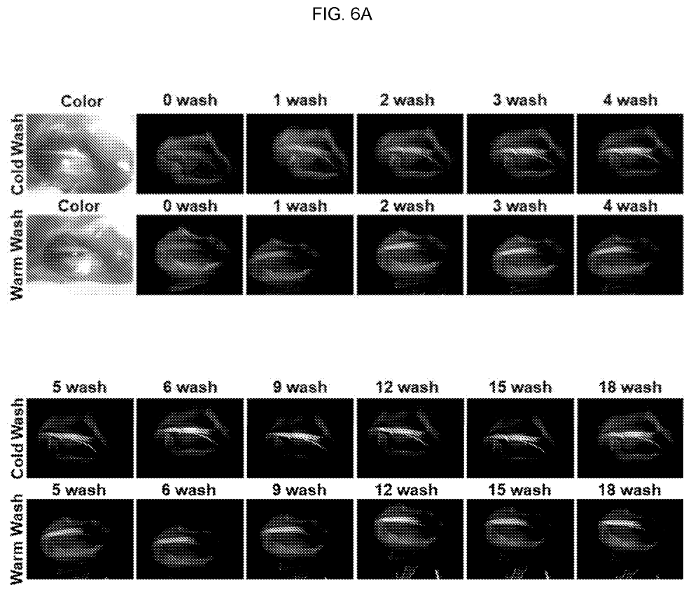

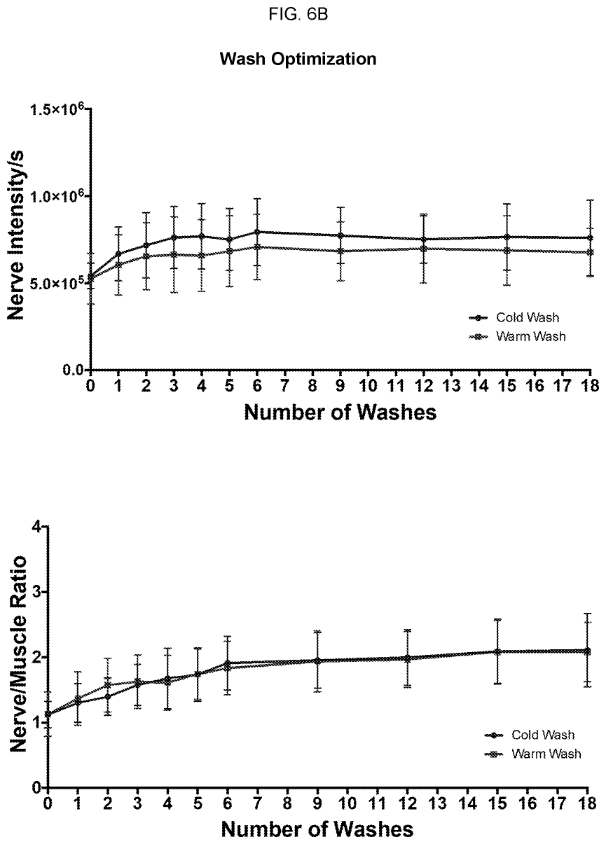

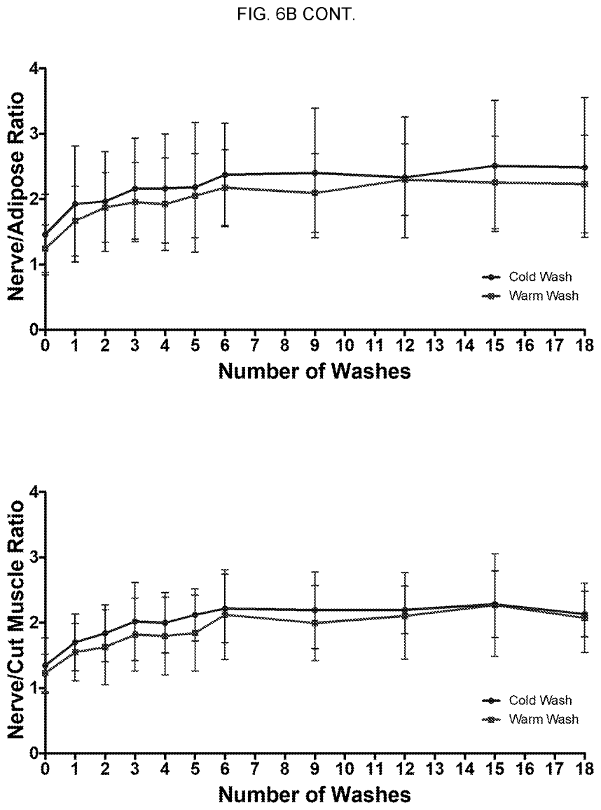

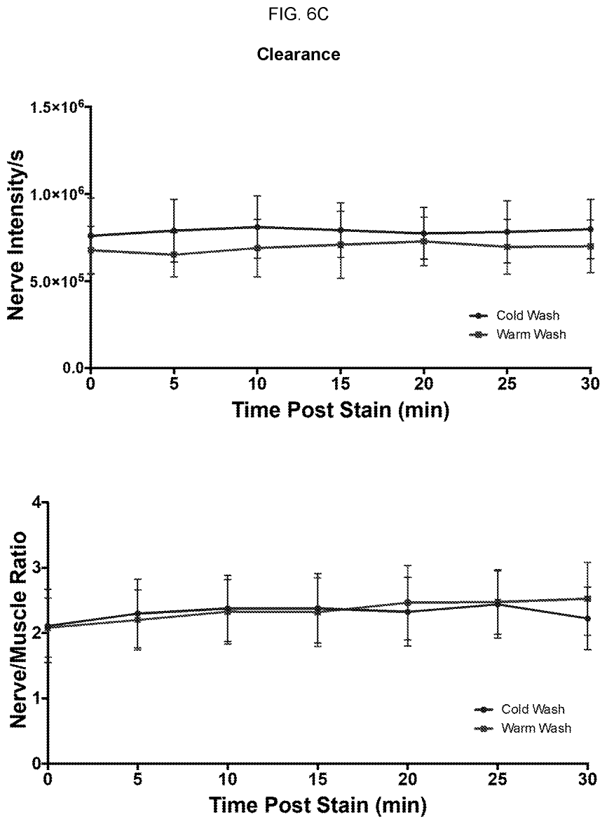

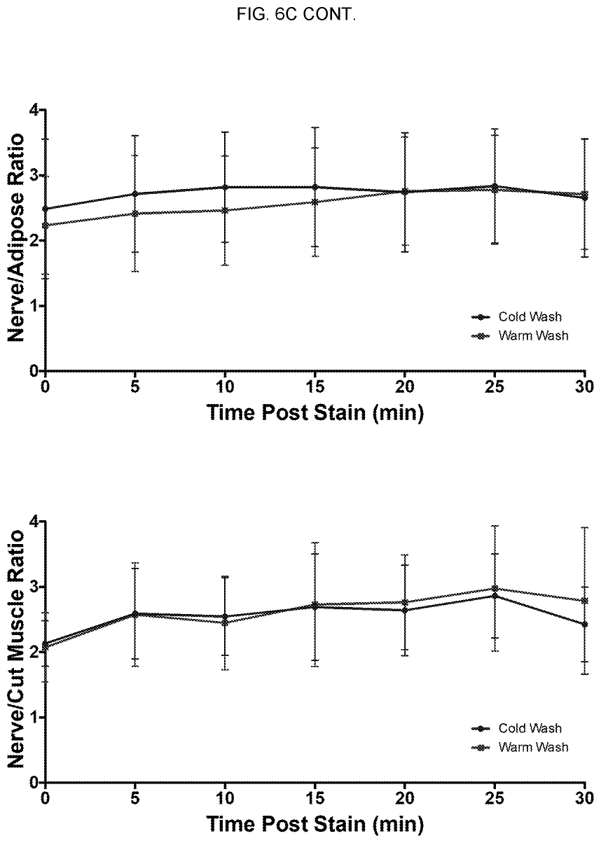

With the fluorophores concentration and incubation time chosen, wash protocol parameters were altered to determine the final amount of washing and wash solution temperature. Warm (37° C.) and cold (4° C.) PBS was used to wash nerve sites stained with a 1 min incubation of 200 μg/mL LGW01-08 in 22% PEO-PPO-PEO triblock formulation. Images were taken throughout the washing process to identify the optimal wash amount (FIGS. 6A-6C). An increase in nerve signal was observed in the first 1 to 2 wash steps followed by a decrease in background muscle and adipose fluorescence in later wash steps (FIG. 6A). Quantified nerve intensities and SBRs agree with this observation for both the cold and warm washes (FIG. 6B). After 6 washes, nerve intensities and SBRs began to level off, with minimal increase out to the final 18^{th} wash step. Thus, 6 total washes were chosen as the most clinically relevant amount of washing. In comparing cold PBS washes to warm PBS washes, cold PBS provided minor yet not significant increases in the nerve signal intensity and nerve to adipose ratio. Therefore, the temperature of the wash solution was not deemed as an important factor in overall wash performance. Following completion of the last wash step, images were collected every 5 min out to 30 min to determine the effect of clearance on nerve signal intensities and SBRs (FIG. 6C). No significant change in nerve signal and slight yet not significant increases in the nerve SBRs occurred during this period, demonstrating the robustness of the final nerve contrast generated from direct gel formulation administration of nerve specific fluorescence.

Rodent Toxicology Testing.

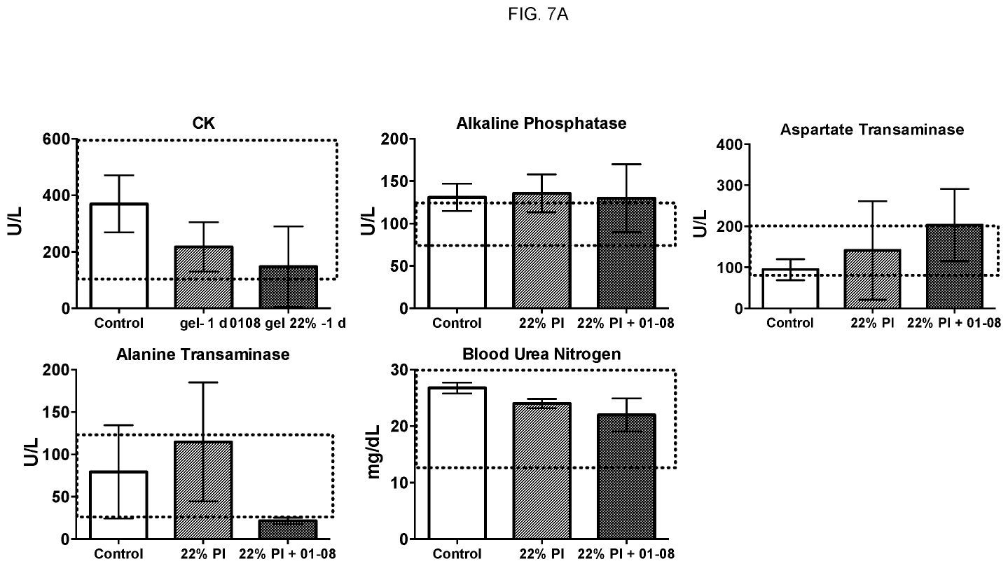

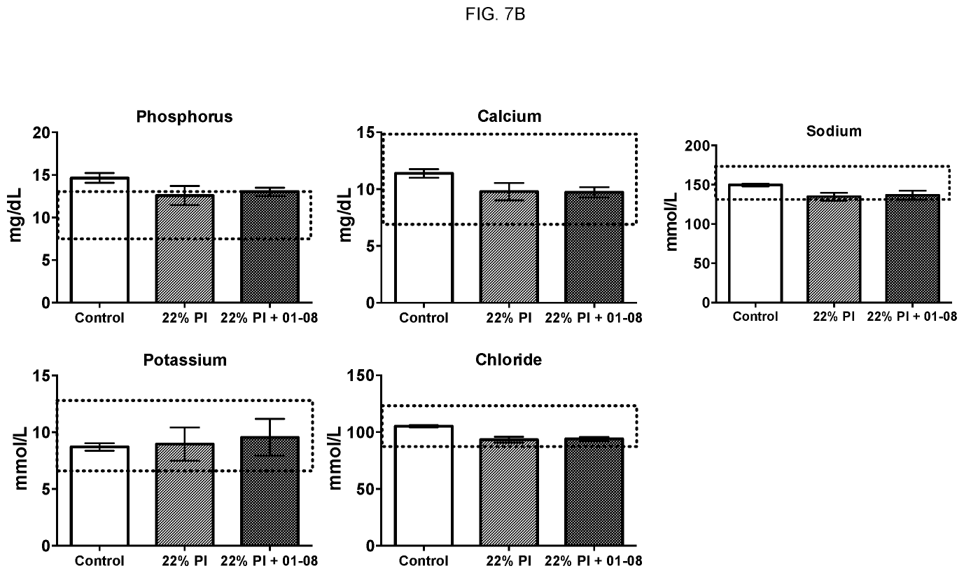

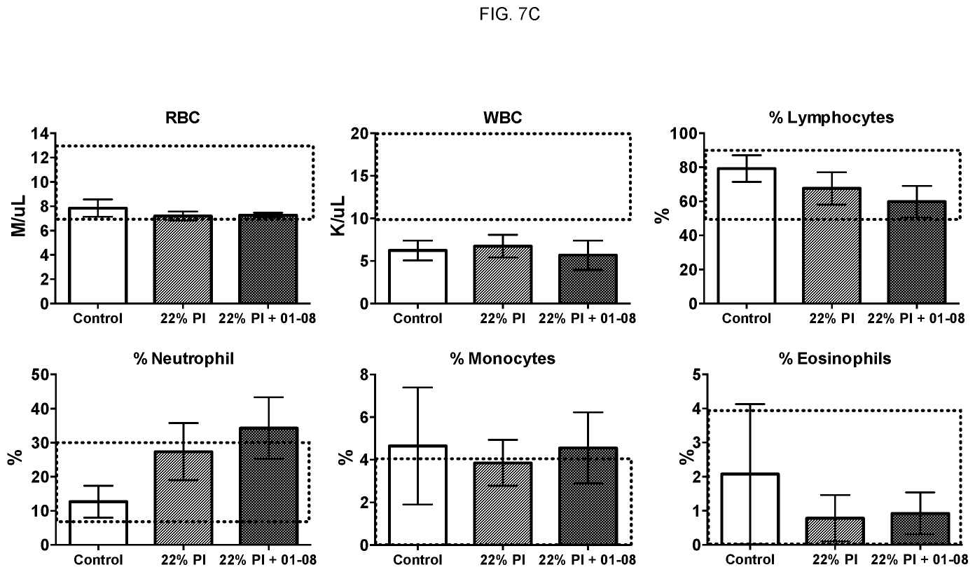

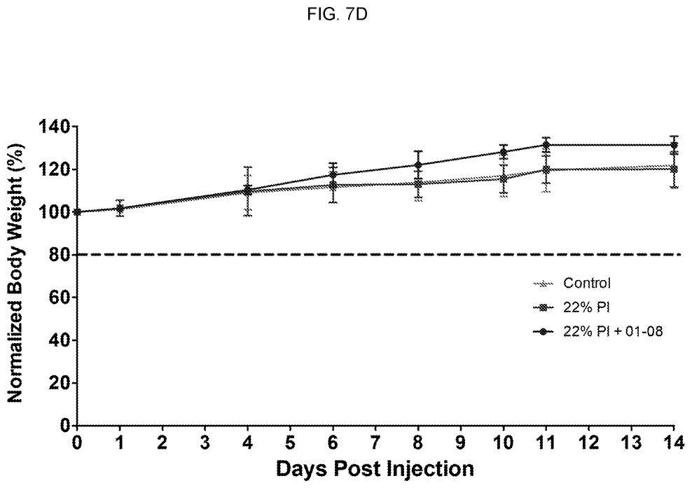

Toxicology testing was performed in rodents for the chosen 22% PEO-PPO-PEO (injected volume 0.5 mL) triblock gel formulation with and without LGW1-08 loading to assess any changes in blood chemistry following administration and screen for any long-term toxicity effects. Subcutaneous injection was utilized as a surrogate to direct administration, with a 1.8 mg/kg dose used in the LGW01-08 administered group or 1× the chosen dose from method optimization. The blank and fluorophore loaded gel formulations were administered to assess any blood chemistry changes and effect on 14-day weight gain as compared to administered control animals (FIGS. 7A-7D). Blood markers for kidney (blood urea nitrogen (BUN) and creatine kinase (CK)) and liver (aspartate transaminase (AST), alkaline phosphatase (ALP), and alanine transaminase (ALT)) toxicity were at normal levels in all cases except for ALP, which was also high in control mice (FIG. 7A). Blood electrolyte amounts were all within the normal range except for phosphorus levels in the control group (FIG. 7B). Hematological analysis for white blood cell (WBC) count, red blood cell (RBC) count, % lymphocytes, % neutrophils, % monocytes, and % eosinophils returned to normal values except for WBC levels in the control and formulation groups, % neutrophils in the 22% PEO-PPO-PEO triblock with LGW1-08 group, and % monocytes in the control and 22% PEO-PPO-PEO triblock plus LGW1-08 groups (FIG. 7C). Mice displayed normal, healthy weight gain during the 14-day monitoring period following administration (FIG. 7D).

Large Animal Studies.

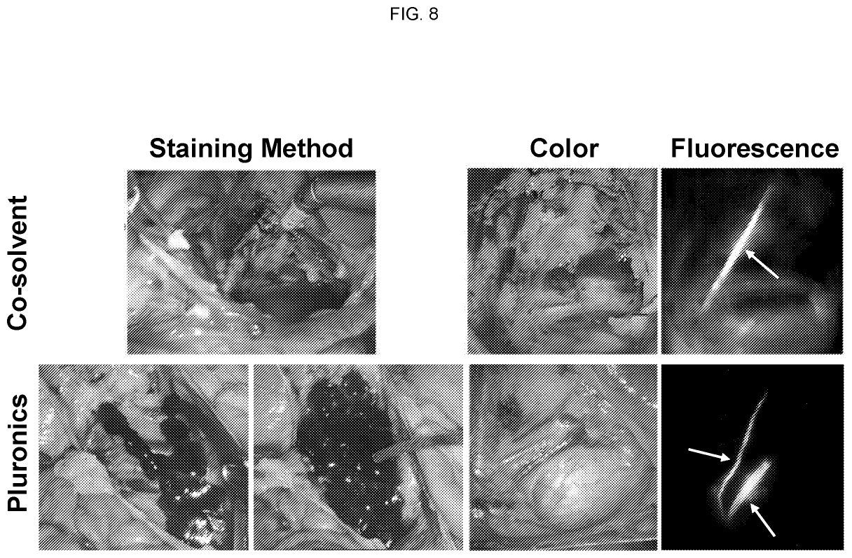

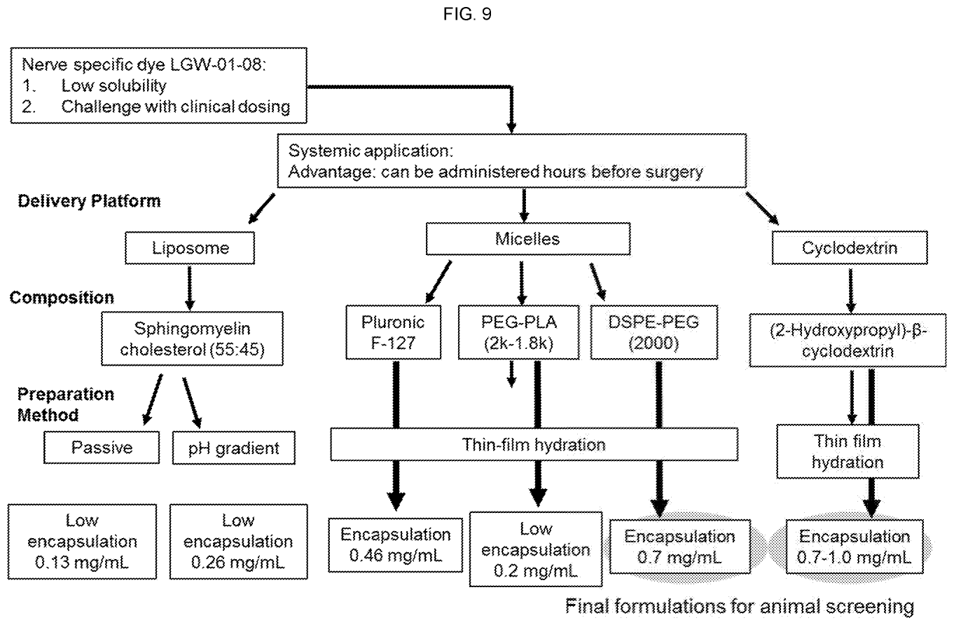

To compare liquid to gel formulation direct administration in a clinically relevant surgical model, swine iliac plexus nerves were stained via direct administration of Oxazine 4 formulated in the co-solvent and PEO-PPO-PEO triblock formulations (FIG. 8). During staining with co-solvent formulation the surrounding tissue had to be tented in order to collect runoff from the near vertical surface of the iliac plexus, generating significant pooling and difficulty controlling the stained area. Resulting fluorescence images, while allowing for clear identification of the main iliac nerve, contained significant background signal from areas where the stain solution had pooled. During staining with the PEO-PPO-PEO triblock formulation, formulated dye was easily applied via syringe through some surgical tubing passed through the assist port of the daVinci system as a liquid and immediately gelled upon contact with the iliac plexus nerve site tissue. Resulting fluorescence images allowed clear identification of the iliac nerve as well as some adjacent buried nerve tissue.

Discussion

In the present example, clinically viable gel formulations were tested using the nerve specific contrast agent LGW01-08 in order to develop a platform for enabling robust and effective direct administration methodology for nerve targeted FGS. The formulations tested, Na Alginate and F127 Pluronic®, were chosen for their viscosity enhancement characteristics, clinical approval status, and beneficial fluorophore solubilization and tissue penetration properties (Table 1). Na Alginate and PEO-PPO-PEO triblock represent different classes of viscosity enhancement, with Na Alginate solidifying at colder temperatures and PEO-PPO-PEO triblock solidifying at warmer temperatures such as body temperature. Na Alginate has been reported to provide excellent coverage on the applied surface and enhanced permeability via a thin hydrogel film that forms via the compound's interaction with the divalent metal ions such as Ca⁺² present in living tissue (Lee & Mooney. Prog Polym Sci 37, 106-126 (2012); Bouhadir et al. Biomaterials 22, 2625-2633 (2001)). PEO-PPO-PEO triblock's unique gelling characteristics, with a liquid-to-gel transition occurring when temperatures increase above a lower critical gelation threshold, make it attractive for maintaining its solidified state when applied within the body. Additionally, its surfactant characteristics improve its fluorophore solubilizing and tissue penetration potential (Nie et al. Int J Nanomedicine 6, 151-166 (2011); Diniz et al. J Mater Sci Mater Med 26, 153 (2015); Suntornnond et al. Sci Rep 7, 16902 (2017)). Additionally, both of these formulations have been reported to provide good basis for foam formulations such as those used clinically during RP like FloSeal (Baxter, Deerfield, Ill.) (Stolzenburg et al. Journal of endourology 24, 505-509 (2010); Liatsikos et al. in Endoscopic Extraperitoneal Radical Prostatectomy 135-142 (Springer, 2007); Unosson et al. J Biomed Mater Res B Appl Biomater 104, 67-77 (2016)). The range of concentrations of each of these formulating agents was chosen to span the transition from liquid at the lower end of the concentrations to solid at the higher end of the concentrations when left at room temperature.

The initial range of viscosity enhancers in formulation were screened in vivo using the direct administration methodology previously analyzed for liquid based formulations. The resulting nerve fluorescence was compared to the co-solvent formulation to ensure no significant loss in nerve contrast or signal intensity occurred (FIGS. 3A, 3B). No significant loss in fluorescence signal vs. the co-solvent formulation was observed except in the highest concentrations of each viscosity enhancer. Fluorescence signal at all concentrations provided positive nerve SBRs, which were equivalent to the co-solvent formulation. The decrease in signal intensities at the higher concentrations of viscosity enhancer were likely due to a lower release rate of the fluorophore in the more solidified gels these concentrations created. To further test the viscosity of these formulations, the formulation ranges were tested for tissue spread at varying degrees of tilt to assess tissue hold and ease of application (FIG. 4). From these tests it was determined that the middle concentrations of each viscosity enhancer (6.5% Na Alginate and 22% PEO-PPO-PEO triblock) were most clinically viable, with the lowest concentration allowing significant spread and the highest inhibiting the ability to initially spread onto the tissue. Additionally, from these tests it was determined that the unique gelling characteristics of PEO-PPO-PEO triblock made it best suited for initial application in the surgical field and subsequent hold for the duration of staining, and thus 22% PEO-PPO-PEO triblock was chosen as the final base gel formulation.

With the base gel formulation chosen and its viscosity characteristics determined, further testing of the fluorophore dose, staining incubation time, and washing protocol were needed to generate a complete gel formulation direct administration method. 200, 50, and 25 μg/mL concentrations of LGW1-08 in 22% PEO-PPO-PEO triblock were used to stain nerve sites with a 1- or 5-min incubation time to assess the interplay between fluorophore concentration and incubation time in efforts to obtain high nerve signal intensity and SBR values as well as minimize the time required to stain (FIGS. 5A, 5B). 50 μg/mL represents the chosen concentration for the direct administration method using liquid formulations and what was used in all initial testing experiments, while the 200 μg/mL concentration represents the maximum dose for remaining beneath the microdose requirements for eIND studies (Barth & Gibbs. Theranostics 7, 573-593 (2017); (ed. F.a.D.A. US Department of Health and Human Services, Center for Drug Evaluation and Research) (Washington D.C., USA; 2006)). The 25 μg/mL concentration was tested to assess any improvements in nerve SBR from decreased dose. No significant change in nerve contrast and SBR values was observed among the different staining conditions, however nerve signal intensities were positively correlated to changes in concentration and incubation time. Thus the 200 μg/mL concentration and 1 min incubation times was chosen as final dose and stain time due to the tradeoff between a shortened staining protocol and resulting nerve signal.

Following staining parameter analysis, several wash conditions were tested to determine the final wash solution and amount of washing for gel formulation application. Warm (37° C.) and cold (4° C.) PBS were tested as wash solutions through a series of flushes applied to stained nerve sites, with images collected during and following the wash protocol (FIGS. 6A-6C). Both temperature wash solutions saw a steady increase in nerve intensity values in the first two wash steps and nerve SBRs values in the first 6 wash steps. The increased nerve signal is likely due to the washing process resolubilizing fluorophore in the wash solutions and allowing for further diffusion into the nerve tissue, while improvements in nerve SBRs likely represent further removal of nonspecifically bound fluorophore. It appears that for both wash solution temperatures, 6 flushes removed the majority of non-specific stain, with further washing providing marginal improvements in nerve SBR. Thus 6 wash steps were chosen as the final washing amount, bringing the overall staining protocol time down to 1-2 minutes. No major differences were observed between the warm and cold wash solutions ability to improve contrast, with the only consistent change being the increased nerve signal from cold wash solution. This result could be due to the cold temperature wash providing improved ability to liquify any leftover PEO-PPO-PEO triblock formulation and allowing for improved secondary staining during washing. These findings suggest wash solution temperature is not an important factor for wash performance, but wherever possible cold wash solution should be utilized. Additionally, clearance assessment following completion of the washing protocol saw no loss in nerve signal and slight increases in nerve SBRs out to 30 minutes. These results suggest that no significant loss in nerve signal or SBRs will occur during the course of a typical nerve sparing procedure.

The final gel formulation was compared to the co-solvent formulation in large animal screening experiments as a more clinically relevant surgical scenario. The swine iliac plexus was stained via direct administration of the two formulations during laparoscopic surgery using the daVinci surgical robot from Intuitive Surgical and fluorescence images were captured via a custom fluorescence channel integrated into the daVinci Si endoscope (FIG. 8). From images taken during the staining process it was apparent the benefit of the gel formulation for improving stain control on the vertical surface of the iliac plexus. While the co-solvent formulation had to be contained via tenting of the surrounding tissue, the PEO-PPO-PEO triblock formulation was easily applied and spread on the tissue as a liquid, but immediately gelled upon contact to be held in place throughout the incubation process. The resulting fluorescence images demonstrate the benefits of the gel formulation in improved nerve contrast and reduced background resulting from pooling of the co-solvent formulation. Additionally, the gel formulation administration enabled visualization of a buried nerve structure adjacent to the iliac nerve, perhaps as a result of increased penetration depth due to increased contact time from the PEO-PPO-PEO triblock gel formulation.

Through systematic optimization and characterization of viscosity enhancer amount, fluorophore concentration, staining incubation time, wash solution, and wash amount the gel formulation and direct administration methodology developed herein can provide bright, highly specific nerve staining that can be applied to many tissue shapes, tilts, and morphologies in under two minutes. The PEO-PPO-PEO triblock-based formulation's unique physical characteristics allow for nerve-specific stain solution to be applied with relative ease as a liquid and then remain in place following near instantaneous gel formation. The resulting platform provides a clinically viable method for fluorescence guided nerve-sparing during RP and an improved route for rapid clinical translation under exploratory IND (eIND) regulations. By requiring much less preclinical toxicology testing, eIND approval is possible with substantially less investment in time and money.

### Example 2. Enhanced Formulation Strategies for Systemic Administration of Nerve-Specific Fluorophores

This example provides clinically viable formulations of NIR oxazine fluorophores for systemic administration to a subject. The formulations provide a strong basis for clinical translation.

Fluorescence guided surgery using near-infrared (NIR) optical imaging technology is capable of wide-field, real-time visualization of targeted tissues intraoperatively with high specificity and sensitivity. Nerve-specific small molecule fluorophores could enable vastly improved nerve identification and thus sparing rates to improve surgical outcomes. A novel NIR oxazine fluorophore, LGW01-08, has demonstrated high nerve specificity following systemic administration in preclinical mouse models, making it an attractive candidate for clinical translation. However, a more clinically viable formulation than the previously utilized co-solvent formulation is needed. A series of clinically relevant formulations for systemic administration of LGW1-08 were developed and screened, including liposomes, polymeric and lipid micelles, nanoparticles and complexation using cyclodextrin. Shah et al. J Control Release 291, 169-183 (2018); Shah et al. J Control Release 253, 37-45 (2017); Doddapaneni et al. J Control Release 220, 503-514 (2015); Hackman et al. Molecular pharmaceutics (2015). A DSPE-PEG micelle formulation and a cyclodextrin formulation were chosen as the most clinically relevant vehicles for administration. Toxicology, pharmacokinetics, and pharmacodynamics parameters were also characterized for the chosen formulations. Clinically relevant imaging time points and doses were identified and maximum tolerated dose, drug release kinetics and biodistribution characteristics were determined for DSPE-PEG micelle and cyclodextrin formulated LGW01-08 injections. The resulting fluorophore formulations provide a strong basis for further testing in large animals and clinical translation.

Materials and Methods.

Contrast Agents and Formulations.

Oxazine 4 perchlorate (Ox4) was obtained from Exciton (Lockbourne, Ohio). LGW1-08 was chosen as the lead compound for development from a library of 64 oxazine derivatives. Ox4 and LGW01-08 were formulated in the previously used co-solvent formulation containing 10% dimethyl sulfoxide (DMSO), 5% Kolliphor EL, 65% serum, and 20% phosphate buffered saline (PBS) (Gibbs-Strauss et al. Molecular imaging 10, 91-101 (2011)) and the four clinically viable formulations including micelles, liposomes and cyclodextrin. Distearyl-phosphatidylethanolamine-PEG2000 (DSPE-PEG) micelle, F-127 micelle, liposome, and (2-Hydroxypropyl)-β (HP-β) cyclodextrin formulations were prepared as described above. Ox4 and LGW01-08 formulations were characterized for drug loading using multiscan spectrum spectrophotometer (Thermo Fischer, Waltham, Mass.). Triplicate samples were prepared for quantification by diluting the formulated fluorophores 100-fold in 10% triton solution and analyzed at 638 nm.

Animals.

Approval for the use of all animals in this study was obtained from the Institutional Animal Care and Use Committee (IACUC) at Oregon Health and Science University (OHSU). Male CD-1 mice weighing 22-24 g were purchased from Charles River Laboratories (Wilmington, Mass.). Prior to surgery, mice were anaesthetized with 100 mg/kg ketamine and 10 mg/kg xylazine (Patterson Veterinary, Devens, Mass.) administered intraperitoneally (IP).

Formulation Nerve-Specificity Screening.

Ox4 and LGW01-08 solubilized in each formulation were screened in mice following systemic administration for nerve specificity. 200 nmol of LGW01-08 in 100 μL of the four prepared formulations were administered intravenously to n=3 mice 4 hours before nerve imaging. The four hour fluorophore to imaging window had been previously shown to provide the highest nerve to background tissue fluorescence for Oxazine 4 and several other nerve specific fluorophores. Gibbs-Strauss et al. Molecular imaging 10, 91-101 (2011); Park et al. Theranostics 4, 823-833 (2014); Barth & Gibbs. Theranostics 7, 573-593 (2017). Uninjected animals were used for all control images (n=3 mice or 12 nerve sites/formulation).

Maximum Tolerated Dose Studies.

The maximum tolerated dose (MTD) was determined in mice for DSPE-PEG micelle and cyclodextrin formulated LGW01-08 administered intravenously. The MTD was determined as half of the lowest acutely lethal dose (i.e., dose limiting toxicity [DLT]) in mice, with injected doses starting at 12 mg/kg. If a dose was found to be acutely lethal following systemic administration, the dose was decreased by half until all mice survived the systemic administration dose (i.e. the DLT). The MTD was defined as half of the DLT. Following quantification of the MTD, two cohorts of mice per formulation were administered formulated LGW1-08 for blood chemistry analysis and 14-day weight monitoring (n=5 mice per cohort per formulation). The blood chemistry cohort of mice were euthanized 24 hours following systemic administration and blood was collected into lithium heparin tubes via cardiac puncture. Blood was also collected into lithium heparin tubes via cardiac puncture following completion of the 14-day weight monitoring studies. All blood samples were sent to IDEXX laboratory (Veterinary Diagnostic, Portland, Oreg.) for standard blood chemistry analysis. The evaluated blood markers included blood urea nitrogen (BUN), creatinine kinase (CK), alanine transaminase (ALT), aspartate transaminase (AST), and alkaline phosphatase (ALP). Hematological analysis assessed white blood cell (WBC) count, red blood cell (RBC) count, Hematocrit (HC), % neutrophils, % lymphocyte, % monocyte, % eosinophil, and % basophil. The electrolytes assessed include phosphorus, calcium, sodium, potassium, and chloride. The weight monitoring cohort of mice were weighed on 1, 2, 3, 5, 7, 9, 11, and 14 days following systemic administration of LGW01-08.

Pharmacokinetics, Biodistribution, and Dose Response.

Drug release pharmacokinetics (PK) and biodistribution (BioD) studies were completed in mice systemically administered DSPE-PEG micelle, cyclodextrin or co-solvent formulated LGW01-08 for equivalent comparison, explained as follows. Formulated LGW1-08 was injected at a 2 mg/kg dose and blood collection was completed at the time points of 0, 0.5, 1, 2, 4, 8, and 24 hours post injection (n=5 mice per time point per formulation). Following blood collection via cardiac puncture, fluorescence and color images were acquired of the brain, lungs, liver, stomach, intestines, pancreas, spleen, kidney, bladder, brachial plexus, and sciatic nerves (n=3 mice per time point per formulation). Additionally, brain, lung, heart, liver, and kidney tissues were collected for liquid chromatography mass spectroscopy (LCMS/MS) analysis (n=5 mice per time point per formulation). The pharmacokinetic parameters were calculated from blood uptake kinetics data by non-compartmental analysis using Phoenix 64 (Certara, Princeton, N.J.), including the area under the concentration versus time curve from 0 to ∞ (AUC0-∞), plasma volume of distribution (Vd), clearance (CL) and half-life (t1/2).

Following PK studies to assess the maximum nerve signal to background ratio (SBR) for LGW1-08 in each formulation (2 hours), dose response pharmacodynamic (PD) studies were completed in mice using a dose range up to the MTD: 0.1, 0.2, 0.3, 0.6, 1, 1.5, 2, 2.5, and 3 mg/kg (n=3 mice per dose per formulation).

Intraoperative Fluorescence Imaging Systems. A custom-built small animal imaging system capable of real-time color and fluorescence imaging was used to acquire rodent in vivo images. The imaging system consisted of a QImaging EXi Blue monochrome camera (Surrey, British Columbia, CA) for fluorescence detection with a removable Bayer filter for collecting co-registered color and fluorescence images. A PhotoFluor II light source (89 North, Burlington, Vt.) was focused onto the surgical field through a liquid light guide and used unfiltered for white light illumination. For fluorescence excitation, the PhotoFluor II was filtered with a 620±30 nm bandpass excitation filter. Resulting fluorescence was collected with a 700±37.5 nm bandpass emission filter for image collection. All filters were obtained from Chroma Technology (Bellows Falls, Vt.). Camera exposure times ranged from 10-2000 ms for fluorescence image collection.

Intraoperative Nerve and Biodistribution Imaging and Image Analysis.

Nerve specific contrast and tissue biodistribution intensities were assessed for all initial testing, PK, BioD, and PD studies using the intraoperative fluorescence imaging systems to collect images of the nerves and surrounding tissue as well as the additional tissues for biodistribution assessment. Additional unstained control animals were imaged to assess tissue autofluorescence.

Custom written MatLab code was used to analyze the tissue specific fluorescence where regions of interest (ROIs) were selected using the white light images. These ROIs were then analyzed on the co-registered matched fluorescence images permitting assessment of the mean tissue intensities and nerve signal to background ratios. Intensity measurements were divided by the exposure time to obtain normalized intensity per second measurements.

Statistical Analysis.

Significant differences between nerve SBR means were evaluated using a one-way ANOVA followed by a Fisher's LSD multiple comparison test with no assumption of sphericity using the Geisser-Greenhouse correction to compare all mean nerve to background tissue ratios. The a value was set to 0.05 for all analyses. Results were presented as mean±standard deviation (S.D.). The pharmacokinetic parameters for all groups were compared by one-way ANOVA with Dunnett's Multiple Comparison post-test at a p-value of 0.05. All statistical analysis was performed using GraphPad Prism (La Jolla, Calif.).

Results.

Initial nerve specificity testing.

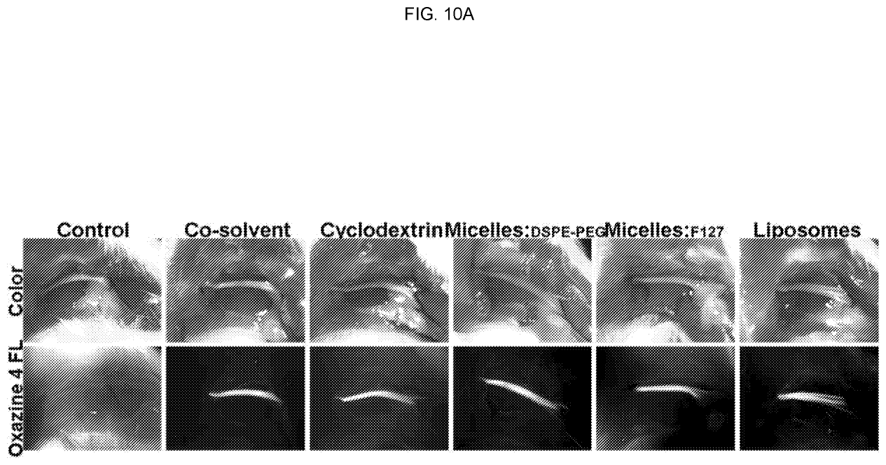

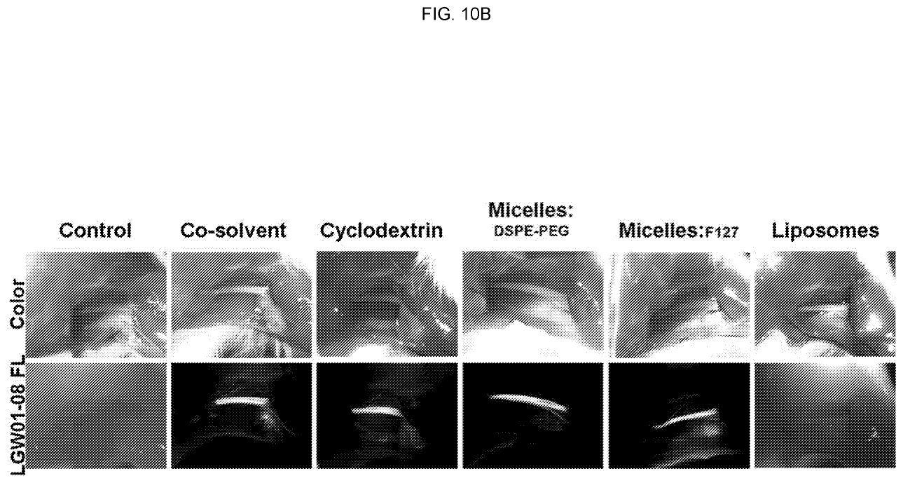

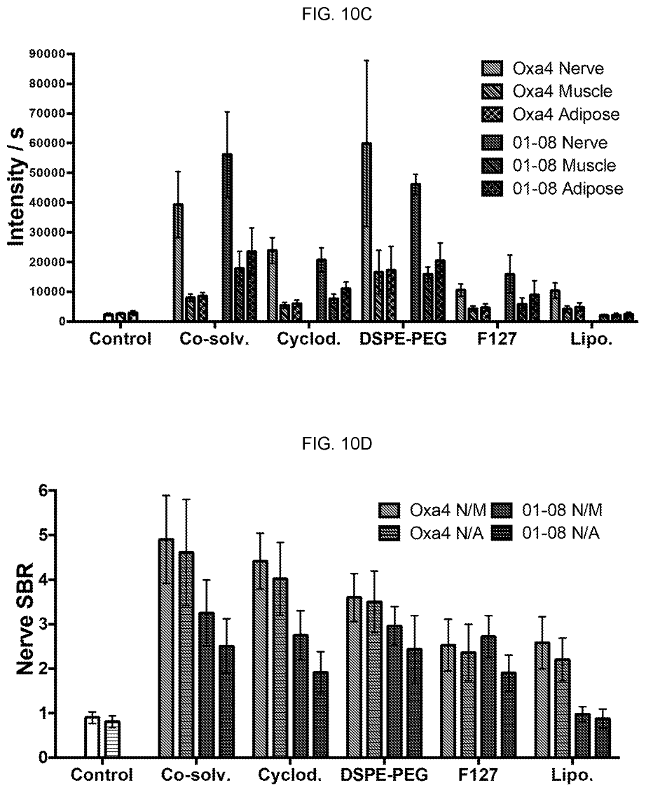

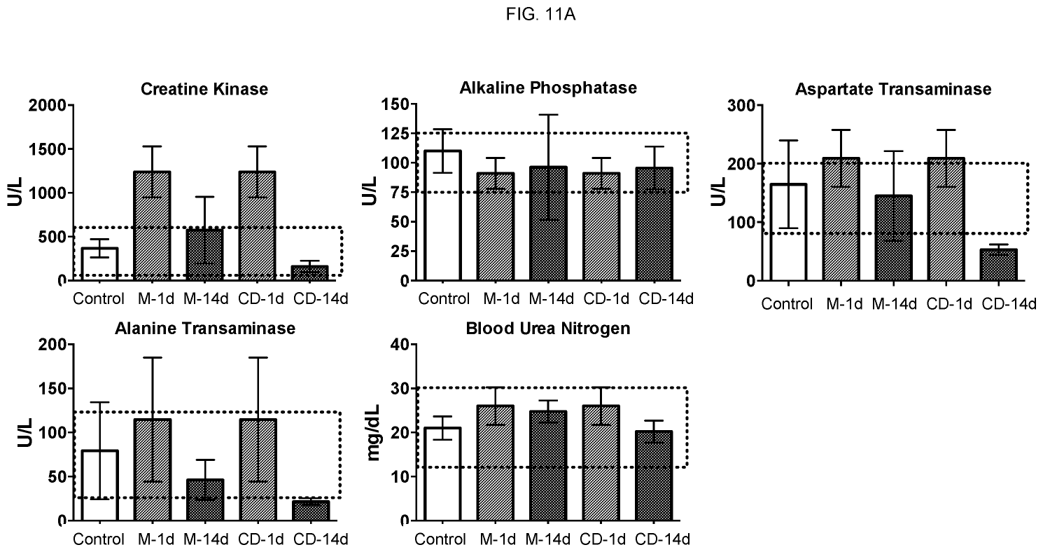

Clinically relevant formulation strategies were tested as viable alternatives to the co-solvent formulation for systemic administration of nerve contrast agents for FGS. Cyclodextrin, F127 micelles, DSPE-PEG micelles, and liposomes were used to solubilize Oxazine 4 and LGW01-08 nerve-specific fluorophores for injection using the previously published dose and imaging window for nerve-specific oxazine fluorophores (Park et al. Theranostics 4, 823-833 (2014); Barth & Gibbs. Theranostics 7, 573-593 (2017)). The resulting nerve contrast was imaged and compared to the co-solvent formulation (FIGS. 10A, 10B). Tissue fluorescence signal intensities varied between the tested formulations, with DSPE-PEG micelles providing the highest intensity nerve signal, which was roughly equivalent to the nerve signal intensity provided by the co-solvent formulation for both Ox4 and LGW01-08 (FIG. 10C). Notably, only the cyclodextrin and DSPE-PEG micelle formulations provided statistically equivalent nerve to muscle SBRs to the co-solvent formulations for Oxazine 4. While the cyclodextrin, DSPE-PEG micelle, and F127 micelle formulations provided equivalent nerve to muscle SBRs to the co-solvent formulations for LGW01-08 (FIG. 10D). Based on these results, the DSPE-PEG micelle formulation was chosen for further characterization and testing.

Formulation composition, stability, and clinical status. The formulations tested herein were chosen for their ability to encapsulate clinically relevant concentrations of LGW1-08 (1-10 mg/mL), stability, FDA approval status, clinical relevance and advantageous tissue uptake characteristics (Table 2). DSPE-PEG and cyclodextrin formulated LGW01-08 was demonstrated to be stable for at least 48 hours in solution and can be freeze dried for long term storage. This is a vast improvement over co-solvent formulated LGW1-08 which is stable for less than 30 minutes and has no freeze drying capability. Additionally, both the DSPE-PEG micelles and cyclodextrin formulations possess FDA approval.

TABLE 2
Clinically viable formulation composition, stability, freeze-drying characteristics, and
regulatory status

| | | Solution | | Freeze-drying | Regulatory |
|---|---|---|---|---|---|
| Formulation | Composition | stability | | capability | status |
| Cosolvent | 10% DMSO, 5% Kolliphor, | <30 | min | No | None |
| | 85% 75/25 Serum/Buffer | | | | |
| Liposome | Sphingomyelin/cholesterol | 48 | h | Yes - with | FDA Approved |
| | | | | cryoprotectant | |
| Micelle | DSPE-PEG (2K) | 72 | h | Yes - with/without | FDA Approved |
| | | | | cryoprotectant | |
| Micelle | Pluronic F-127 | 48 | h | Yes - with/without | FDA Approved |
| | | | | cryoprotectant | |
| Micelle | PEG-PLA (2 k-1.8 k) | 120 | h | Yes - with/without | Under clinical |
| | | | | cryoprotectant | development in |
| | | | | | US, approved in |
| | | | | | Korea |
| Cyclodextrin | (2-Hydroxypropy1)-β- | 72 | h | Yes - without | FDA Approved |
| | cyclodextrin | | | cryoprotectant | |

Rodent Toxicology Testing.

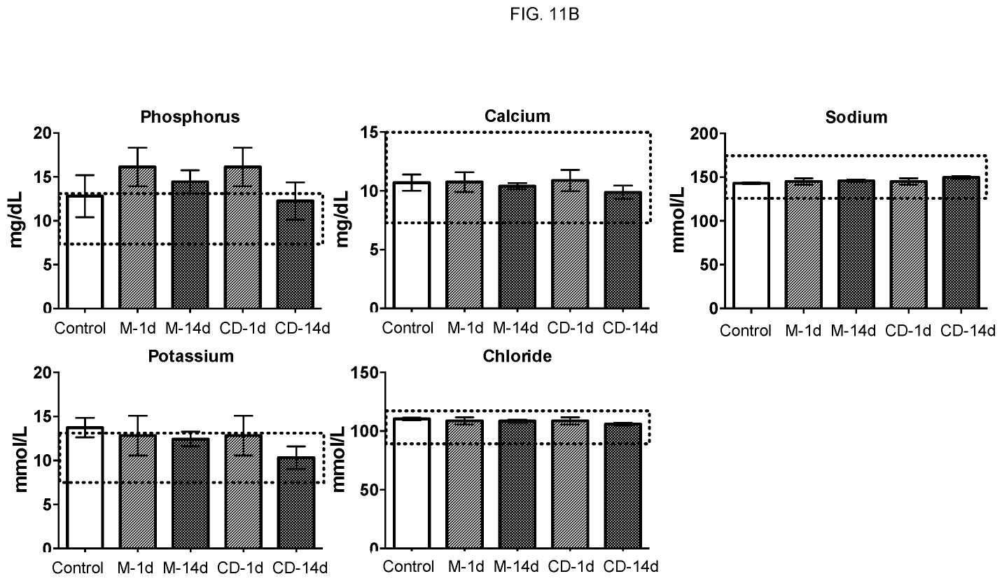

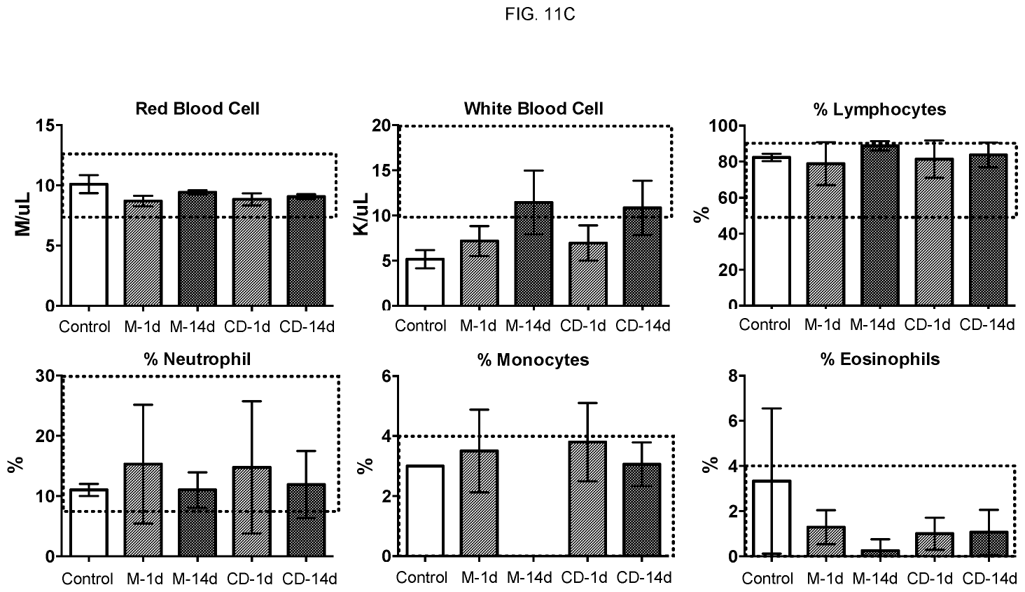

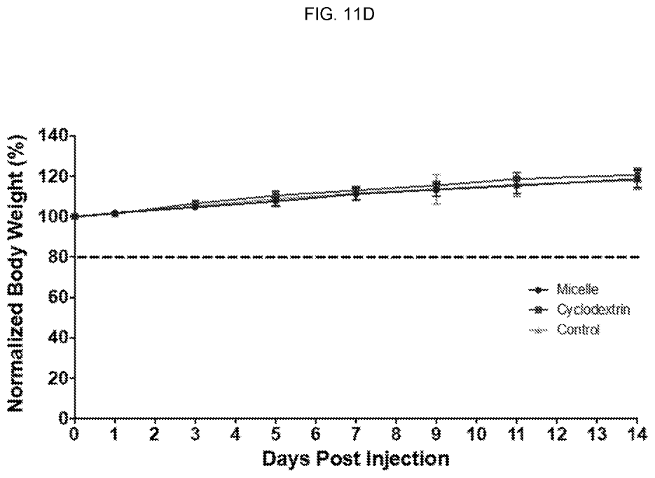

The MTD in mice was determined to be 3 mg/kg for the DSPE-PEG micelle and cyclodextrin formulated LGW1-08, where a maximum dose of 12 mg/kg was tested as the starting dose to determine acute DLT following systemic administration. The MTD of LGW1-08 formulated in DSPE-PEG micelle and cyclodextrin was administered to assess any blood chemistry changes and effect on 14-day weight gain (FIGS. 11A-11D). Elevation in CK and AST markers were seen one day post systemic administration, while BUN and ALT were at the level of control mice. All blood markers returned to normal levels 14 days after systemic administration (FIG. 11A). Phosphorus levels were elevated one day post systemic administration following administration of LGW1-08 in both formulations. Phosphorus levels returned to normal for cyclodextrin formulated LGW1-08 at 14-days post injection but remained slightly elevated for DSPE-PEG micelle formulated LGW1-08. Notably, the control group also showed phosphorus levels at the high end of the normal range. All other blood electrolytes levels were within the normal range both one and 14-days post systemic administration (FIG. 11B). WBC counts were diminished one day after systemic administration but returned to normal levels 14-days post administration for DSPE-PEG micelle formulated LGW01-08. All other hematological markers were in the normal range both one and 14-days after systemic administration of LGW1-08 formulated in DSPE-PEG micelles and cyclodextrin (FIG. 11C). Notably, 0% monocytes was measured 14-days post injection for the DSPE-PEG micelle formulated fluorophore cohort; however this is within the normal range. Following administration of the MTD in either formulation, mice displayed normal, healthy weight gain during the 14-day monitoring period (FIG. 11D).

Pharmacokinetics, Biodistribution, and Imaging Time Course Studies.

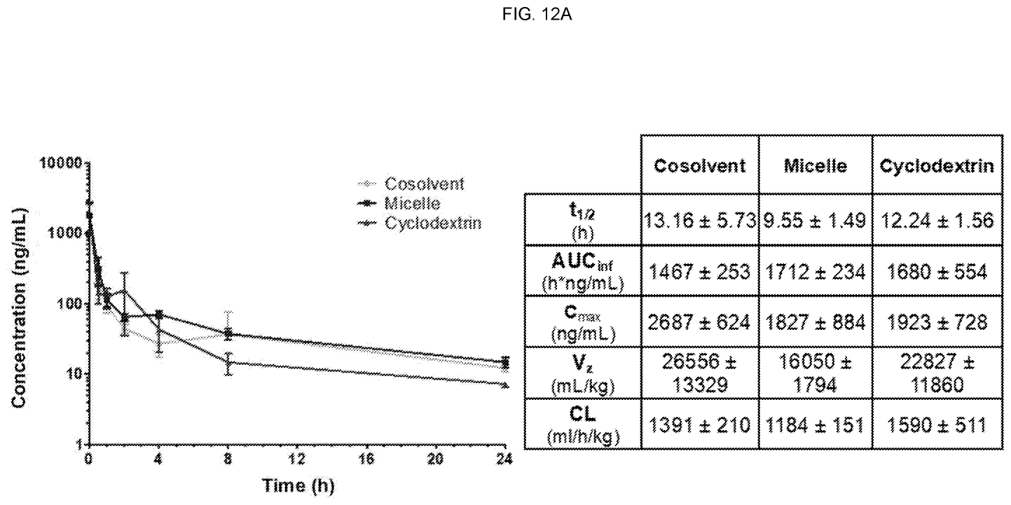

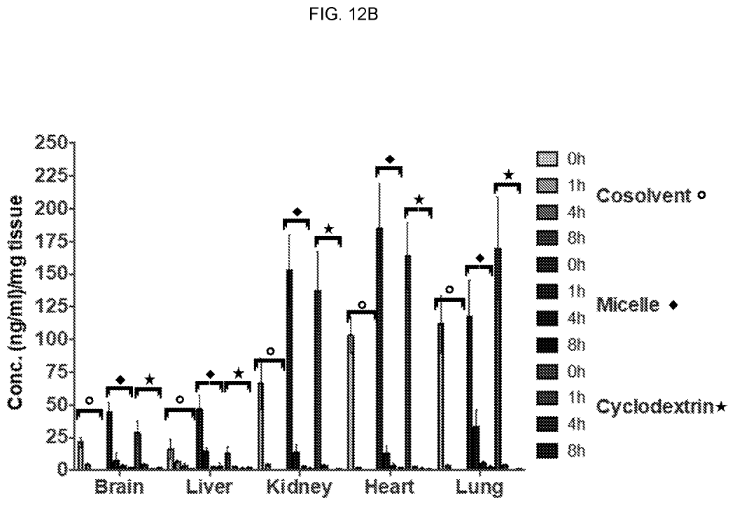

LGW1-08 pharmacokinetics (PK), biodistribution (bioD), and imaging time course studies were performed using a 2 mg/kg dose. Fluorophore uptake was determined using LCMS/MS analysis of murine blood and tissue collected at 0, 0.5, 1, 2, 4, 8, and 24-hours post systemic administration to quantify kinetics and tissue biodistribution (FIGS. 12A, 12B). LGW1-08 blood concentrations decreased quickly in all formulations with cyclodextrin showing decreased release at 2 hours and increased release at 4-, 8-, and 24-hour time points as compared to the co-solvent formulation. Micelles showed increased release at 2- and 4-hour time points as compared to the co-solvent formulation. The elimination half-life (t_{1/2}), total area under the plasma concentration-time curve (AUC_{inf}), and total body clearance of drug from plasma (CL) values calculated for each formulation showed no statistical difference between the three formulations. The terminal phase volume of distribution (V_{z}) values were 1.65 times higher for the co-solvent and cyclodextrin formulation compared to the DSPE-PEG micelle formulation (FIG. 12A). The half-life is directly proportional to V_{z}, with co-solvent and cyclodextrin half-life times 1.3-1.4 times longer than the DSPE-PEG micelle half-life. Tissue LGW1-08 concentrations followed a similar trend to blood concentration levels, with 90-99% of LGW01-08 cleared from the tissue by 4 hours post injection and high levels in highly blood perfused tissues like heart, lung and brain falling rapidly after injection as expected under normal clearance conditions (FIG. 12B).

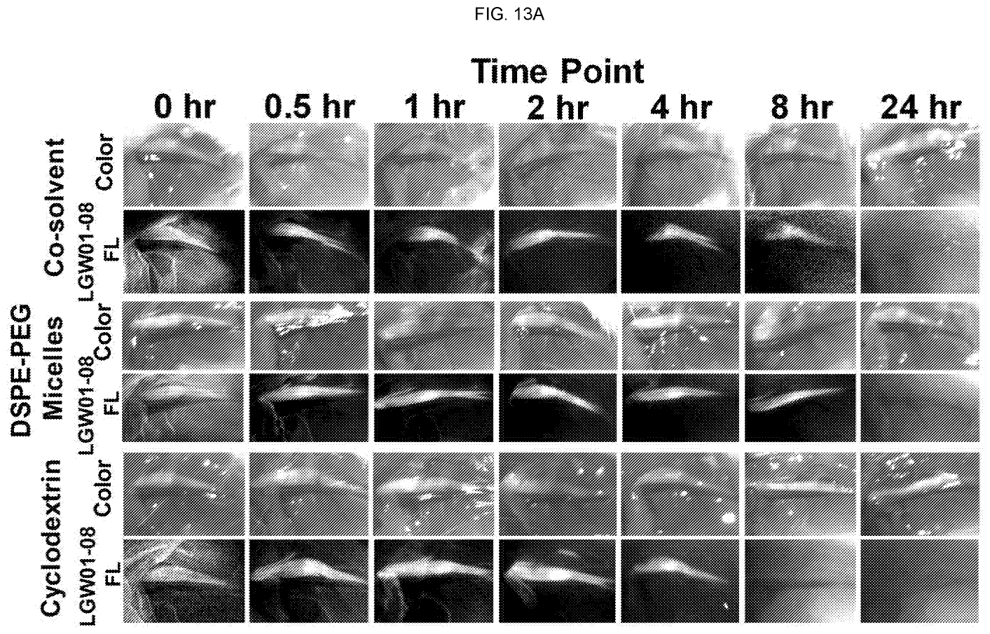

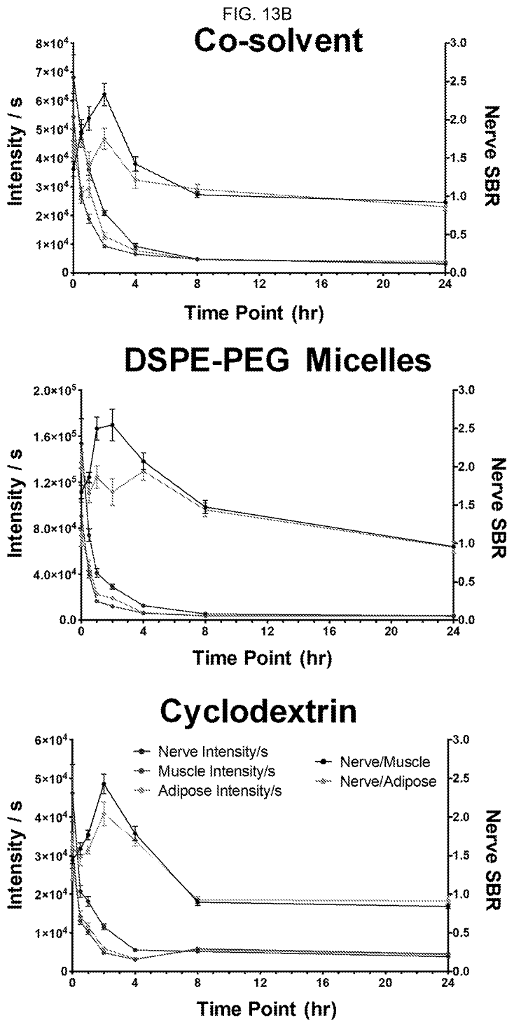

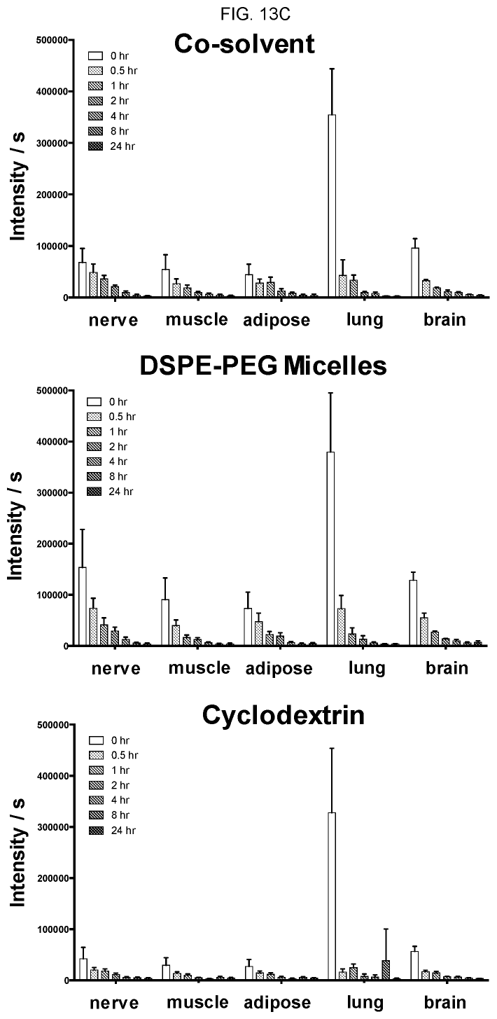

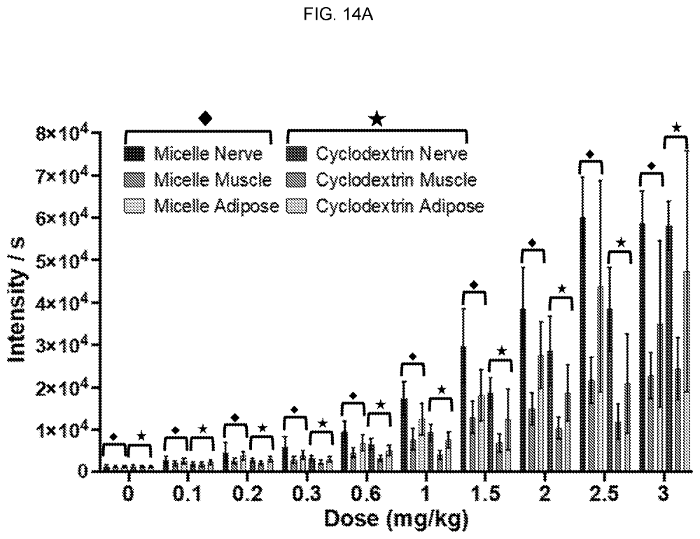

Brachial plexus and sciatic nerve images were also collected to assess nerve contrast and identify the most clinically viable imaging time point (FIG. 13A). Interestingly, nerve uptake occurred immediately following systemic administration and nerve contrast was observed as soon as 30 min following injection which was maintained for at least 8 hours for both the co-solvent and DSPE-PEG micelle formulations (FIG. 13A). Nerve fluorescence signal intensities decreased significantly in the first two hours following injection, with fluorescence signal intensities falling close to baseline autofluorescence levels 24 hours after systemic administration of LGW1-08 in all formulations (FIG. 13B). Background tissue (muscle and adipose) fluorescence signal intensities decreased at a greater rate in the first two hours post LGW1-08 administration, resulting in increasing nerve to muscle and nerve to adipose SBRs. Nerve to muscle ratios were roughly equivalent for all formulations out to four hours post systemic administration, with DSPE-PEG micelle formulation showing a faster increase and more sustained maximum contrast ratio at the one- and two-hour time points compared to the co-solvent and cyclodextrin formulations. Nerve to adipose ratios were roughly equivalent between all formulations. Nerve SBRs peaked at two hours post injection and thus the two-hour time point was chosen as the most clinically viable imaging time point (FIG. 13B).

In addition to nerve, muscle, and adipose tissues, brain and lung tissues were also imaged and fluorescence signal intensities were measured for biodistribution and kinetics assessment (FIG. 13C). Notably, fluorescence signal intensities in the lung and brain tissues followed a similar trend to the nerve tissue fluorescence intensities, with signal decreasing significantly in the first two hours for both formulations. Substantial uptake in lung tissue immediately following injection was evident and in line with the results from the LCMS/MS tissue biodistribution analysis (FIGS. 12B, 13C).

Dose Ranging Pharmacodynamics Imaging Studies.

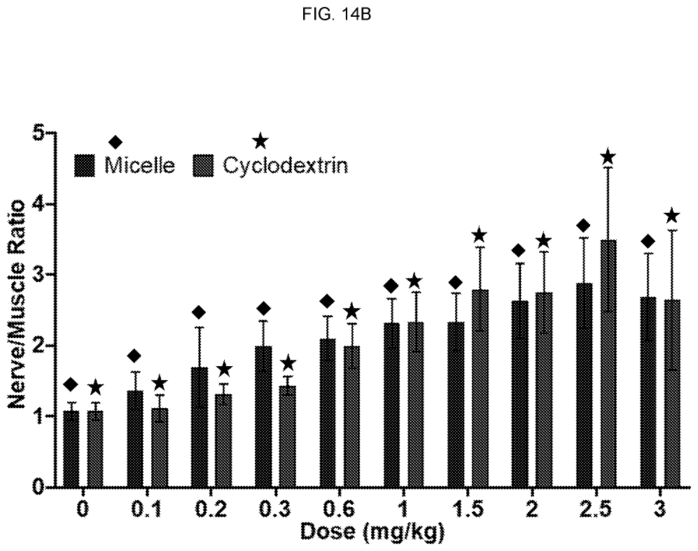

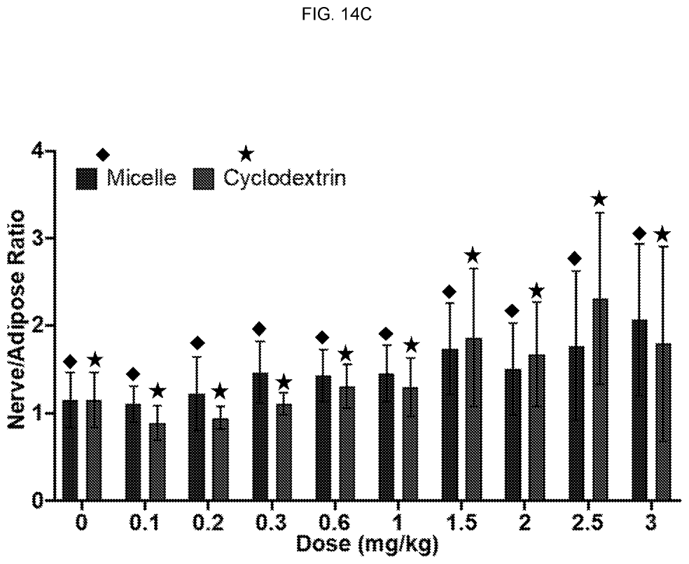

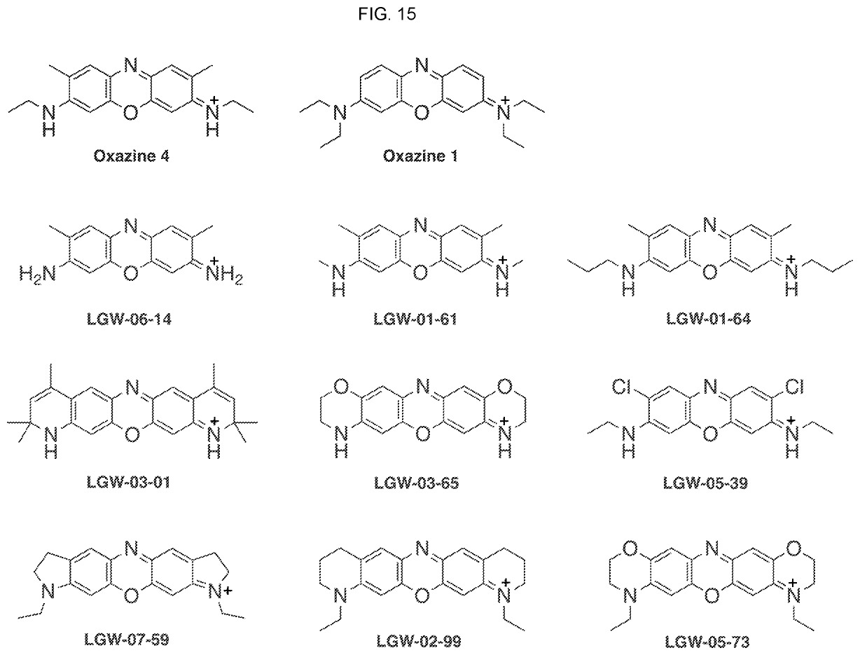

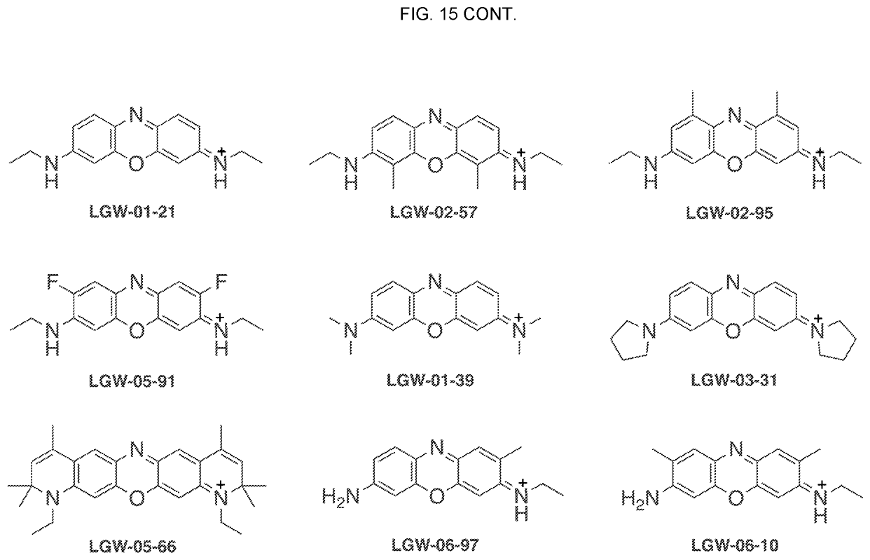

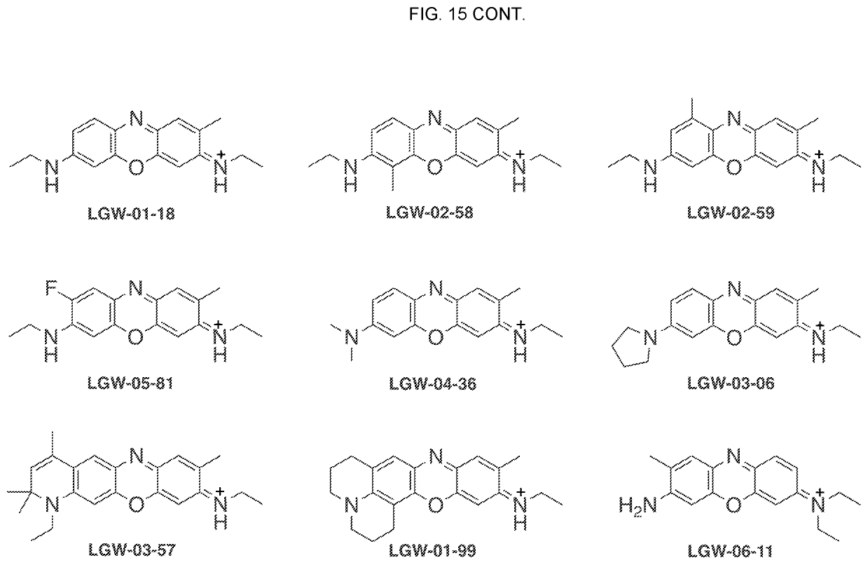

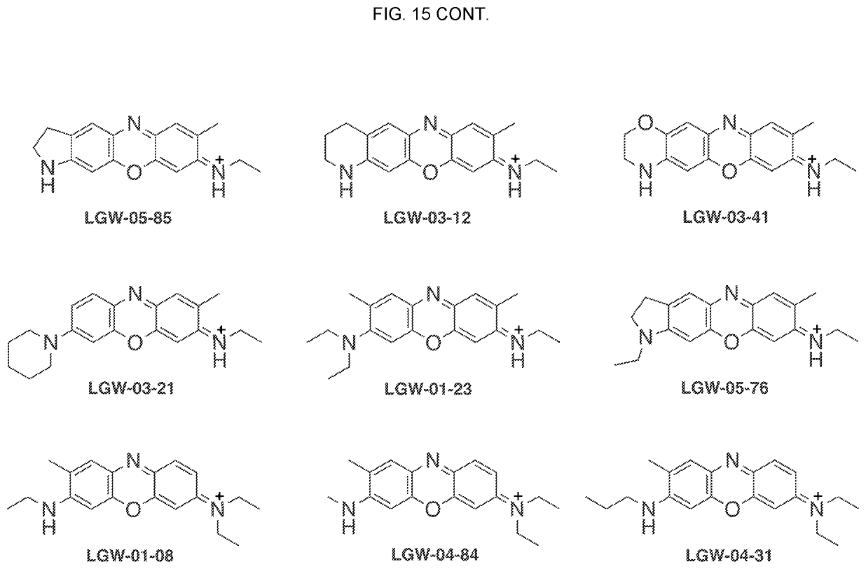

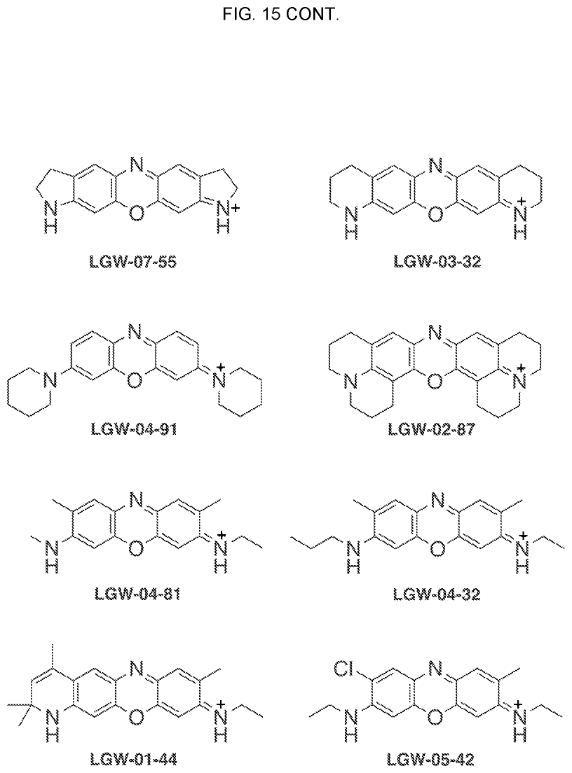

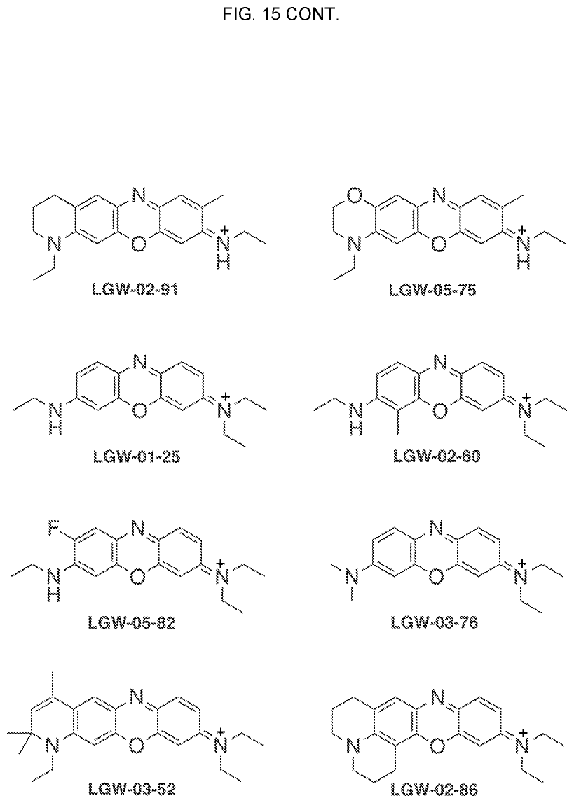

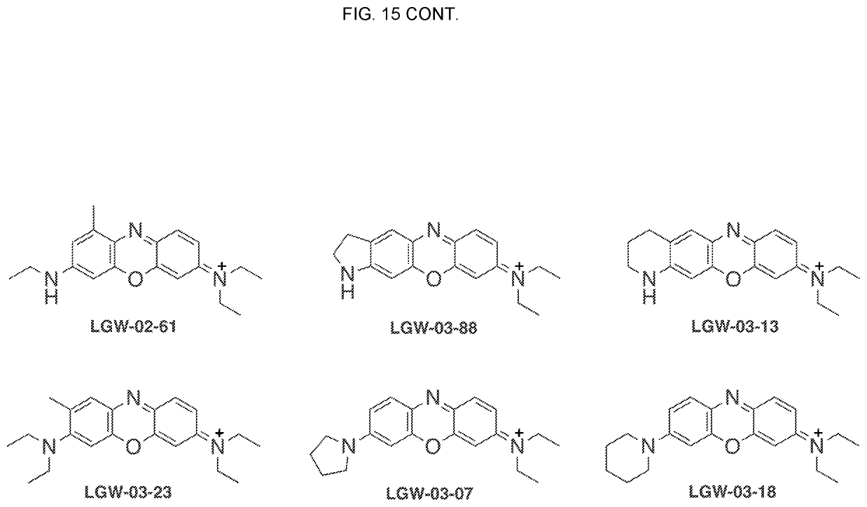

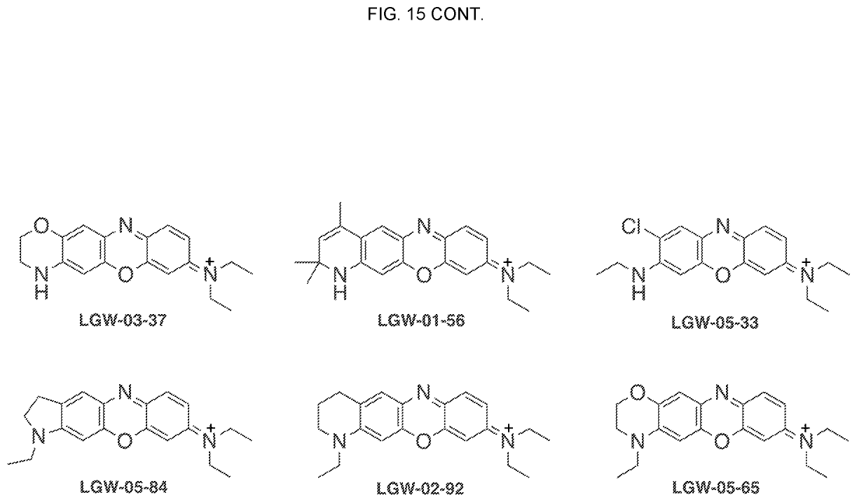

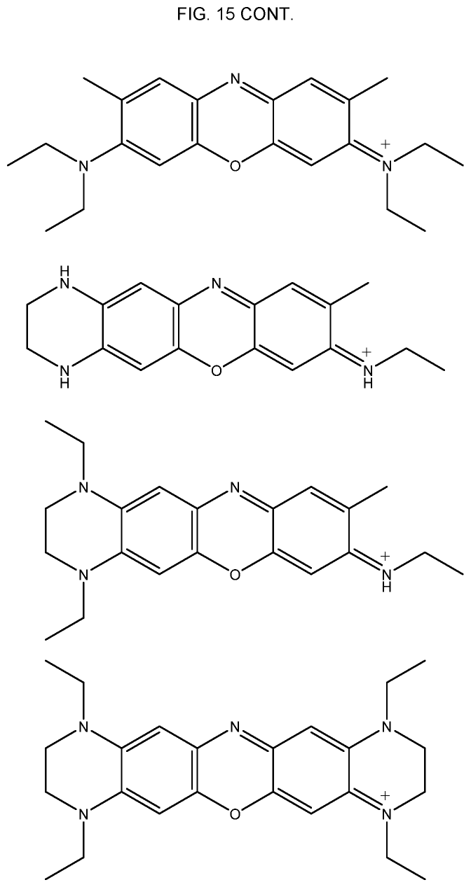

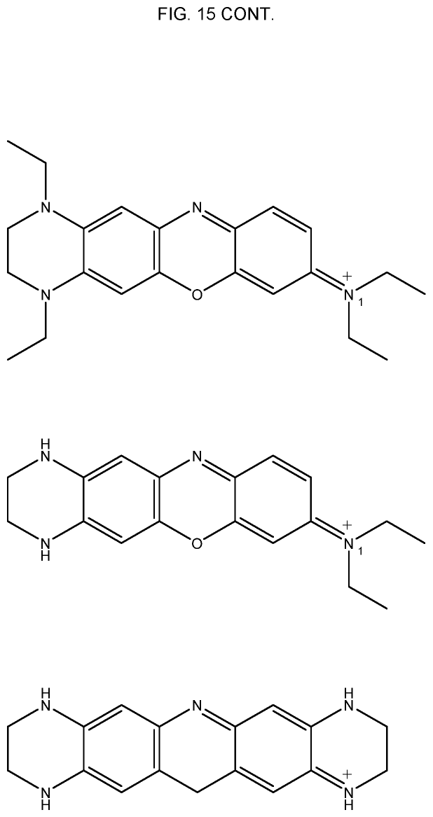

Dose scaling studies were performed to determine the most clinically viable dose for injection of LGW01-08 in the DSPE-PEG micelle and cyclodextrin formulations. 0.1, 0.2, 0.3, 0.6, 1, 1.5, 2, 2.5, and 3 mg/kg doses were administered and brachial plexus and sciatic nerve fluorescence images were collected two hours post injection to assess the resulting nerve contrast (FIGS. 14A-14C). Fluorescence intensity values were highest at 2.5 mg/kg, decreased slightly at 3 mg/kg, and were decreased to autofluorescence levels at the 0.1 mg/kg dose (FIG. 14A). Nerve to muscle background ratios increased as dose increased to 0.6 mg/kg and nerve to adipose background ratios increased as dose increased to 1.5 mg/kg. No significant difference in nerve SBRs was observed across all doses tested (FIGS. 14B-14C).

Discussion.

Clinically relevant formulation strategies were tested for systemic administration of a NIR nerve specific fluorophore, LGW01-08, with the ultimate goal of determining a viable formulation for clinical translation of this promising agent for nerve sparing FGS. A range of formulations with clinical approval or under clinical development (Table 2) were chosen to compare nerve labelling performance to the previously utilized co-solvent formulation with both Oxazine 4 and LGW01-08 (FIGS. 10A-10D). The formulations were chosen for their high stability, storage potential, and clinical use. A liposomal formulation (Sphingomyelin), several micelle formulations (DSPE-PEG, PEO-PPO-PEO triblock, PEG-PLA), and a cyclodextrin formulation (HP-β) were tested. Importantly, all formulations had improved stability when compared to the co-solvent formulation, with all solution stabilities greater than or equal to 48 hours (Table 2). Additionally, the tested formulations can all be freeze dried, allowing for extended storage potential. With all chosen formulations possessing either FDA approval or in the process of clinical development, these formulations represent excellent candidates as vehicles for clinical administration of LGW1-08. Upon initial screening of each formulation, the DSPE-PEG micelle and cyclodextrin formulations provided the best performance, with only slightly lower or greater fluorescence signal intensities compared to the co-solvent formulation for Oxazine 4 and LGW1-08 injections and equivalent nerve SBRs compared to the co-solvent formulation for Oxazine 4 and LGW01-08 injections and equivalent nerve SBRs compared to the co-solvent formulation for Oxazine 4 and LGW01-08 injections (FIGS. 10A-10D). Thus, the DSPE-PEG micelle and cyclodextrin formulations were chosen and utilized in further testing.

With clinically viable formulations chosen, aspects of the formulated fluorophore's toxicity, pharmacokinetics, and dose response were tested. Maximum tolerated dose (MTD) studies were performed to determine the dose-limiting toxicity of the formulated fluorophore and any changes in blood chemistry levels and long-term weight gain following systemic administration in mice (FIGS. 11A-11D). Most blood chemistry markers remained within their normal range one and 14 days following injection at the determined MTD of 3 mg/kg for both formulations (FIGS. 11A-11C). Blood markers for heart (Creatine Kinase, CK) and liver (aspartate transaminase, AST) toxicity were elevated at one day post injection. However, these markers are often elevated due to the stressed caused by injections and values returned to normal levels 14 days post injection (FIG. 11A). Nonetheless, an extended exposure toxicology study should be performed, with complete histological analysis of each tissue following a regiment of regular doses. Among blood electrolytes, phosphorus levels were high at both time points following injection for micelle formulated LGW1-08 and at one day following injection for cyclodextrin formulated LGW1-08, however these levels were not significantly higher than the control group. All other electrolyte levels were normal following injection (FIG. 11B). In hematological analysis, white blood cell levels were low at baseline in the control and all formulation groups one day following injection, however these levels returned to the normal range 14 days post injection (FIG. 11C). Additionally, mice showed normal continual weight gain when monitored out to 14 days following injection and no abnormal behavioral or physiological symptoms were observed (FIG. 11D). Therefore, while some blood markers showed elevated levels shortly after injection, no major or long-term toxicity issues were detected for the DSPE-PEG micelle/LGW01-08 or cyclodextrin/LGW01-08 drug formulation.

Pharmacokinetics and biodistribution studies were completed with a 2 mg/kg dose at 0, 0.5, 1, 2, 4, 8, and 24-hour time points to evaluate drug release kinetics and tissue uptake in comparison to the co-solvent formulation and to determine the most clinically relevant imaging time point (FIGS. 12A, 12B). LGW01-08 blood concentration levels and nerve, muscle and adipose tissue fluorescence intensity values agreed well with one another and matched closely between the co-solvent and DSPE-PEG micelle formulations (FIGS. 12A, 13B). All drug kinetic parameters were roughly equivalent between both formulations except for the volume of distribution during terminal phase (V_{z}), which was 1.65 times higher in the co-solvent formulation injected animals and 1.4 times higher in the cyclodextrin formulation injected animals as compared to the DSPE-PEG micelle formulation (FIG. 12A). However, the values for all three formulations are between 5-100 L/kg, indicating a high V_{z}, which signifies that the inherent nature of LGW01-08 favors higher tissue partition compared to blood (Smith et al. J Med Chem 58, 5691-5698 (2015)). Tissue biodistribution levels followed the expected trend for intravenously administered drugs, with high levels in highly blood perfused organs at initial time points followed by rapid decrease as LGW01-08 starts getting eliminated from the body (FIG. 12B). The fluorescence imaging biodistribution analysis data agreed with these findings (FIG. 13C). Fluorescence imaging time-course studies found that two hours post injection was the time point that generated the highest nerve contrast for all formulations (FIGS. 13A, 13B). Interestingly, the micelle formulation provided a slightly faster increase to peak nerve to muscle ratios at the one hour time point, which was sustained at two hours post injection. Two hours was chosen as the most clinically relevant imaging time point for systemic administration of DSPE-PEG micelle formulated LGW01-08.

With a clinical imaging time point chosen, pharmacodynamics dose ranging studies were performed to determine the clinical dose for FGS. Doses ranged from half the MTD at 1.5 mg/kg up to the MTD at 3 mg/kg (FIGS. 14A-14C). No obvious trend was observed in nerve SBRs as doses were varied with nerve to muscle ratios slightly increased at 3 mg/kg and signal intensities followed a rough increase as dose increased, however intensities at 3 mg/kg were slightly lower that at 2.5 mg/kg. Due to the tradeoff between signal intensities and nerve SBR values and to avoid any potential toxicity issues from dosing at the MTD, 2.5 mg/kg was chosen as the most clinically viable dose for both formulations.

In the present study, clinically relevant formulations strategies were explored to determine a viable formulation for clinical translation of nerve specific FGS enabled via systemic administration of the oxazine fluorophore LGW1-08. The chosen DSPE-PEG micelle and cyclodextrin formulations provided equivalent probe delivery and nerve contrast to the previously utilized co-solvent formulation, are stable for 72 hours and capable of freeze drying for long-term storage, and is FDA approved for a number of applications. Oerlemans et al. Pharmaceutical research 27, 2569-2589 (2010); Allen & Cullis. Advanced drug delivery reviews 65, 36-48 (2013); Gidwani & Vyas. Biomed Res Int 2015, 198268 (2015); Ottinger et al. Curr Top Med Chem 14, 330-339 (2014); di Cagno. Molecules 22 (2016). The work presented herein has yielded a platform for clinical translation of this promising nerve specific fluorophore and provided insight into the pharmacological properties of DSPE-PEG micelle and cyclodextrin formulated oxazine compounds.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a significant decrease in SBR at a site of surgical intervention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; 18% of the stated value; 17% of the stated value; 16% of the stated value; 15% of the stated value; 14% of the stated value; ±13% of the stated value; ±12% of the stated value; 11% of the stated value; 10% of the stated value; ±9% of the stated value; 8% of the stated value; 7% of the stated value; 6% of the stated value; 5% of the stated value; 4% of the stated value; 3% of the stated value; 2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A gel-based formulation for tissue imaging comprising (i) a fluorophore, and (ii) 5-10% sodium alginate and/or 18-26% PEO-PPO-PEO triblock copolymer.

2. The gel-based formulation of claim 1 wherein the fluorophore is an oxazine derivative.

3. The gel-based formulation of claim 2 wherein the oxazine derivative is LGW1-08.

4. The gel-based formulation of claim 1 comprising 50 μg/mL fluorophore.

5. The gel-based formulation of claim 1 comprising 200 μg/mL fluorophore.

6. The gel-based formulation of claim 1 comprising 6.5% sodium alginate.

7. The gel-based formulation of claim 1 comprising 22% PEO-PPO-PEO triblock copolymer.

8. A method of directly applying the gel-based formulation of any of claims 1-7 comprising applying the gel-based formulation to an exposed nerve.

9. The method of claim 8 wherein the applying is during radical prostatectomy.

10. The method of claim 8 further comprising washing the applied gel-based formulation from the nerve.

11. The method of claim 10 wherein the washing comprises 5-7 flushes.

12. A formulation for systemic administration tissue imaging comprising (i) a fluorophore, and (ii) a DSPE-PEG micelle and/or cyclodextrin.

13. The formulation of claim 12 wherein the fluorophore is an oxazine derivative.

14. The formulation of claim 13 wherein the oxazine derivative is LGW1-08.

15. The formulation of claim 12 comprising a DSPE-PEG micelle with the fluorophore encapsulated at 0.5-0.9 mg/mL.

16. The formulation of claim 12 comprising a DSPE-PEG micelle with the fluorophore encapsulated at 0.7 mg/mL.

17. The formulation of claim 12 comprising cyclodextrin with the fluorophore encapsulated at 0.5-1.2 mg/mL.

18. The formulation of claim 12 comprising cyclodextrin with the fluorophore encapsulated at 0.7-1.0 mg/mL.

19. A method of staining a nerve or tissue comprising systemically administering a formulation of any of claims 12-18 to a subject during an operative procedure.

20. The method of claim 19 wherein the administering is at dose of 2.5 mg/kg.

21. A method of detecting nerves intraoperatively in a subject undergoing surgery comprising:
directly applying a gel-based formulation comprising (i) a fluorophore, and (ii) 5-10% sodium alginate and/or 18-26% PEO-PPO-PEO triblock copolymer to stain tissue undergoing surgery; and
imaging the stained tissue, thereby detecting nerves intraoperatively in the subject undergoing surgery.

22. The method of claim 21, further comprising washing the tissue with buffer after applying the gel-based formulation and prior to imaging the stained tissue.

23. The method of claim 22, wherein the washing removes unbound fluorophore.

24. The method of claim 23, wherein the buffer is phosphate-buffered saline (PBS).

25. The method of claim 22, further comprising allowing the gel-based formulation to penetrate the tissue for 1-2 minutes prior to the washing.

26. The method of claim 21, wherein risk of iatrogenic injury to the subject undergoing surgery is reduced.

27. The method of claim 21, wherein the surgery is laparoscopic.

28. The method of claim 21, wherein the surgery is performed by a robot.

29. The method of claim 21, wherein the surgery is radical prostatectomy.

30. The method of claim 21, wherein the fluorophore is an oxazine derivative.

31. The method of claim 30, wherein the oxazine derivative is LGW01-08.

32. The method of claim 21, wherein the concentration of the fluorophore is 50 μg/mL.

33. The method of claim 21, wherein the concentration of the fluorophore is 200 μg/mL.

34. The method of claim 21, wherein the percentage of sodium alginate is 6.5%.

35. The method of claim 21, wherein the percentage of PEO-PPO-PEO triblock copolymer is 22%.

36. A method of detecting nerves within ex vivo tissue comprising:
directly applying a gel-based formulation comprising (i) a fluorophore, and (ii) 5-10% sodium alginate and/or 18-26% PEO-PPO-PEO triblock copolymer to stain the ex vivo tissue; and
imaging the stained ex vivo tissue, thereby detecting nerves within the ex vivo tissue.

37. The method of claim 36, further comprising washing the ex vivo tissue with buffer after applying the gel-based formulation and prior to imaging the stained ex vivo tissue.

38. The method of claim 37, wherein the buffer is phosphate-buffered saline (PBS).

39. The method of claim 37, further comprising allowing the gel-based formulation to penetrate the ex vivo tissue for 1-2 minutes prior to the washing.

40. The method of claim 35, wherein the fluorophore is an oxazine derivative.

41. The method of claim 39, wherein the oxazine derivative is LGW01-08.

42. The method of claim 35, wherein the concentration of the fluorophore is 50 μg/mL.

43. The method of claim 35, wherein the concentration of the fluorophore is 200 μg/mL.

44. The method of claim 35, wherein the percentage of sodium alginate is 6.5%.

45. The method of claim 35, wherein the percentage of PEO-PPO-PEO triblock copolymer is 22%.

46. A method of detecting nerves intraoperatively in a subject undergoing surgery comprising:
systemically administering a formulation (i) a fluorophore, and (ii) a DSPE-PEG micelle and/or cyclodextrin to the subject before or during surgery; and
imaging stained tissue undergoing surgery in the subject, thereby detecting nerves intraoperatively in the subject undergoing surgery.

47. The method of claim 46, wherein systemically administering comprises intravenously injecting the subject with the formulation.

48. The method of claim 46, comprising systemically administering the formulation 30 minutes to 4 hours prior to the imaging.

49. The method of claim 46, wherein risk of iatrogenic injury to the subject undergoing surgery is reduced.

50. The method of claim 46, wherein the surgery is laparoscopic.

51. The method of claim 46, wherein the surgery is performed by a robot.

52. The method of claim 46, wherein the fluorophore is an oxazine derivative.

53. The method of claim 52, wherein the oxazine derivative is LGW1-08.

54. The method of claim 46, wherein the fluorophore is encapsulated by the DSPE-PEG micelle at 0.5-0.9 mg/mL.

55. The method of claim 46, wherein the fluorophore is encapsulated by the DSPE-PEG micelle at 0.7 mg/mL.

56. The method of claim 46, wherein the fluorophore is encapsulated by cyclodextrin at 0.5-1.2 mg/mL.

57. The method of claim 46, wherein the fluorophore is encapsulated by cyclodextrin at 0.7-1.0 mg/mL.

58. A kit comprising:
(a) a gel-based formulation comprising (i) a fluorophore, and (ii) 5-10% sodium alginate and/or 18-26% PEO-PPO-PEO triblock copolymer; and/or
(b) a formulation comprising (i) a fluorophore, and (ii) a DSPE-PEG micelle and/or cyclodextrin; and
(c) use instructions for applying the formulation of (a) and/or administering the formulation of (b).

59. The kit of claim 58, wherein the fluorophore is an oxazine derivative.

60. The kit of claim 59, wherein the oxazine derivative is LGW1-08.

61. The kit of claim 58, wherein the concentration of the fluorophore is 50 μg/mL.

62. The kit of claim 58, wherein the concentration of the fluorophore is 200 μg/mL.

63. The kit of claim 58, wherein the percentage of sodium alginate is 6.5%.

64. The kit of claim 58, wherein the percentage of PEO-PPO-PEO triblock copolymer is 22%.

65. The kit of claim 58, wherein the fluorophore is encapsulated by the DSPE-PEG micelle at 0.5-0.9 mg/mL.

66. The kit of claim 58, wherein the fluorophore is encapsulated by the DSPE-PEG micelle at 0.7 mg/mL.

67. The kit of claim 58, wherein the fluorophore is encapsulated by cyclodextrin at 0.5-1.2 mg/mL.

68. The kit of claim 58, wherein the fluorophore is encapsulated by cyclodextrin at 0.7-1.0 mg/mL.
